(19) United States
(12) Patent Application Publication
(10) Pub. No.: US 2012/0142644 A1
(43) Pub. Date: Jun. 7, 2012

(54) METHOD FOR PRODUCING PHYTOSTEROL/PHYTOSTANOL PHOSPHOLIPID ESTERS

(76) Inventors: Jørn Borch Søe, Tilst (DK); Tina Lillan Jørgensen, Silkeborg (DK)

(21) Appl. No.: 13/231,355

(22) Filed: Sep. 13, 2011

Related U.S. Application Data
(63) Continuation-in-part of application No. PCT/IB2010/051339, filed on Mar. 26, 2010.

(30) Foreign Application Priority Data
Mar. 27, 2009 (GB) 0905367.9

(51) Int. Cl. A61K 31/56; C07J 1/00; C12P 7/62; A23L 1/30; C12P 9/00

(52) U.S. Cl. 514/169; 426/531; 435/131; 435/135; 552/502

(57) ABSTRACT

The present invention relates to a method of producing a phytosterol ester and/or a phytostanol ester comprising: a) admixing a phospholipid composition comprising at least between about 10% to about 70% plant phospholipid and at least about 5% water; a lipid acyltransferase; and a phytosterol and/or a phytostanol; and b) separating or isolating or purifying at least one phytosterol ester and/or phytostanol ester from said admixture. The present invention also relates to compositions comprising the phytosterol ester and/or phytostanol ester produced by this method, including foodstuffs and personal care product (cosmetic) compositions.

### FIELD OF THE PRESENT INVENTION

The present invention relates to a process for producing a phytosterol ester and/or a phytostanol ester using a lipid acyltransferase. The present invention further relates to uses of a lipid acyltransferase to produce a phytosterol ester and/or a phytostanol ester.

### BACKGROUND OF THE PRESENT INVENTION

It is well established to incorporate phytosterol esters into food products like mayonnaise and margarine mainly because of its cholesterol lowering effects. The food products enriched with phytosterol esters or phytostanol esters are often called “functional foods” (i.e. enriched margarine). Phytostanol esters and phytosterol esters have also been used in the personal care products (cosmetics) industry. It is more preferable to use sterol esters and/or stanol esters rather than free sterols or stanols in food and other applications because sterol esters and/or stanol esters are more stable.

Sterol esters and/or stanol esters are conventionally produced by a chemical esterification of the corresponding sterol/stanol compounds with fatty acids. Enzymatic procedures for the preparation of sterol esters are known but typically require organic solvents and/or molecular sieves. In known methods for producing sterol ester and/or stanol ester several purification steps are often required before it can be used in certain applications, particularly in food applications.

Consumers and companies are striving for products and production processes which are sustainable, more environmentally friendly and leaner compared with the production of sterol esters and/or stanol esters using chemicals and organic solvent systems.

Therefore one object of the present invention is to provide a more sustainable, environmentally friendly and leaner process for the production of phytosterol esters and/or phytostanol esters.

### SUMMARY ASPECTS OF THE PRESENT INVENTION

Aspects of the present invention are presented in the claims and in the following commentary.

It has surprisingly been found that an efficient and effective method for the production of phytosterol esters and/or phytostanol esters can be achieved by the use of a lipid acyltransferase in an aqueous environment by combining an phospholipid composition comprising at least between about 10% to about 70% plant phospholipid and at least about 5% water with an acyltransferase and a phytosterol and/or phytostanol.

This method provides sustainable, environmentally friendly and leaner process for the production of phytosterol esters and/or phytostanol esters.

### DETAILED ASPECTS OF THE PRESENT INVENTION

According to a first aspect of the present invention there is provided a method of producing a phytosterol ester and/or a phytostanol ester comprising:
a) preparing a reaction composition by admixing a phospholipid composition comprising at least between about 10% to about 70% plant phospholipid; a lipid acyltransferase; and a phytosterol and/or a phytostanol; and optionally water, wherein the reaction composition comprises at least 2% water w/w; and
b) isolating or purifying at least one phytosterol ester and/or phytostanol ester.

According to another aspect of the present invention there is provided a method of producing a phytosterol ester and/or a phytostanol ester comprising:
a) admixing a phospholipid composition comprising at least between about 10% to about 70% plant phospholipid and at least about 2% water; a lipid acyltransferase; and a phytosterol and/or a phytostanol; and
b) isolating or purifying at least one phytosterol ester and/or phytostanol ester from said admixture.

A further aspect of the present invention provides a use of a lipid acyltransferase to produce a phytosterol ester and/or a phytostanol ester in a reaction composition comprising a) a phospholipid composition, comprising at least between about 10% to about 70% plant phospholipids, b) at least about 2% water and c) an added phytosterol and/or a phytostanol.

In a further aspect there is provided a use of a lipid acyltransferase to produce a phytosterol ester and/or a phytostanol ester in a phospholipid composition comprising at least between about 10% to about 70% plant phospholipids and at least about 5% water; wherein a phytosterol and/or phytostanol is added to said phospholipid composition.

The present invention further provides in another aspect a method of producing a foodstuff comprising a phytosterol ester and/or a phytostanol ester, wherein the method comprises the step of adding a phytosterol ester and/or a phytostanol ester obtained by any of the methods and/or uses of the present invention to a foodstuff and/or a food material.

In a yet further embodiment there is provided a method of producing a personal care product (e.g. a cosmetic) comprising a phytosterol ester and/or a phytostanol ester, wherein the method comprises the step of adding the phytosterol ester and/or a phytostanol ester obtained by any of the methods and/or uses of the present invention to a further personal care product (e.g. cosmetic) constituent.

Another aspect of the present invention provides a composition comprising a phytosterol ester and/or a phytostanol ester obtained by any of the methods and/or uses of the present invention.

In a yet further aspect of the present invention there is provided a foodstuff comprising a phytosterol ester and/or a phytostanol ester obtained by any of the methods and/or uses of the present invention.

The present invention further provides a personal care product (e.g. cosmetic) composition comprising a phytosterol ester and/or a phytostanol ester obtained by any, of the methods and/or uses of the present invention and optionally a cosmetic diluent, excipient or carrier.

Preferably the phytosterol and/or phytostanol is added in amount of at least 5% of the reaction composition, overall admixture or overall composition.

In one embodiment preferably the phytosterol ester and/or phytostanol ester is admixed with a foodstuff or food ingredient.

In another embodiment preferably the phytosterol ester and/or phytostanol ester is admixed with a pharmaceutical diluent, carrier or excipient or a cosmetic diluent, carrier or excipient.

Preferably the phytosterol and/or phytostanol comprises one or more of the following structural features:
i) a 3-beta hydroxy group or a 3-alpha hydroxy group; and/or
ii) A:B rings in the cis position or A:B rings in the trans position or C₅-C₆ is unsaturated.

In one embodiment, preferably the phytosterol is selected from the group consisting of one or more of the following: alpha-sitosterol, beta-sitosterol, stigmasterol, ergosterol, campesterol, 5,6-dihydrosterol, brassica sterol, alpha-spinasterol, beta-spinasterol, gamma-spinasterol, deltaspinasterol, fucosterol, dimosterol, ascosterol, serebisterol, episterol, anasterol, avenasterol, clionasterol, hyposterol, chondrillasterol, desmosterol, chalinosterol, poriferasterol, clionasterol, sterol glycosides, and other natural or synthetic isomeric forms and derivatives.

In one embodiment, preferably the phytostanol is selected from the group consisting of one or more of the following: alpha-sitostanol, beta-sitostanol, stigmastanol, ergostanol, campestanol, 5,6-dihydrostanol, brassica stanol, alpha-spinastanol, beta-spinastanol, gamma-spinastanol, deltaspinastanol, fucostanol, dimostanol, ascostanol, serebistanol, epistanol, anastanol, avenastanol, clionastanol, hypostanol, chondrillastanol, desmostanol, chalinostanol, poriferastanol, clionastanol, stanol glycosides, and other natural or synthetic isomeric forms and derivatives.

Suitably, phytostanols for use in the present invention may be obtained from hydrogenation of sterols (see U.S. Pat. No. 6,866,837 for example).

In one aspect the phytosterol and/or phytostanol added to or admixed with the phospholipid composition may be one or more phytosterols, one or more phytostanols or a mixture of at least one phytosterol and at least one phytostanol.

Preferably the phytosterol and/or phytostanol is exogenous (i.e. not naturally occurring) in the phospholipid composition. In other words, the phytosterol and/or phytostanol is added to the phospholipid composition. Hence the term “added phytosterol” or“added phystostanol” as used herein means that the phytosterol and/or phytostanol is an exogenous phytosterol and/or phytosterol which is not naturally present in the phospholipid composition. Even if some phytosterol and/or some phytostanol is naturally present in the phospholipid composition, preferably additional exogenous phytosterol and/or phytostanol is added to or admixed with the phospholipid composition. Suitably in one aspect the amount of phytosterol and/or phytostanol added may be such that the reaction composition, e.g. the reaction admixture and/or the reaction composition, comprises the plant phospholipid and the phytosterol/phytostanol in a 1:1 ratio. In this way neither the phospholipid nor the phytosterol/phytostanol become rate limiting on the reaction.

Preferably the phytosterol and/or phytostanol is added in an amount of at least about 5% (or at least about 10% or at least about 15% or at least about 20%) of the reaction composition or overall admixture or overall composition.

In one aspect the phytosterol and/or phytostanol may be added in an amount of less than about 30%, suitably less than about 25%, suitably less than about 21% of the reaction composition or overall admixture or overall composition.

In one embodiment the phytosterol and/or phytostanol used in the method and uses of the present invention may be a natural source of phytosterols and/or phytostanols such as soybean oil deodorizer distillate (SODD) for example.

Preferably, a lyso-phospholipid is also produced in the method or uses of the present invention.

When a lyso-phospholipid is also produced, preferably the lyso-phospholipid is purified or isolated.

The “phospholipid composition” according to the present invention may be any composition comprising at least between about 10% to about 70% plant phospholipid.

Suitably the phospholipid composition may comprise one or more plant phospholipids. In one embodiment the phospholipid composition is a mixture of two or more, preferably 3 or more, plant phospholipids.

In one embodiment the phospholipid composition comprises between about 10% and about 65%, or between about 10%, and about 50% or between about 10% and about 40% plant phospholipid.

In one aspect the phospholipid composition comprises at least about 10% plant phospholipid, at least about 20% plant phospholipid or at least about 30% plant phospholipid.

In one aspect the phospholipid composition comprises at most about 70% plant phospholipid, at most about 60% plant phospholipid, at most about 50% plant phospholipid or at most about 40% plant phospholipid.

In one embodiment, the “phospholipid composition” according to the present invention may be any composition comprising at least between about 10% to about 70% plant phospholipid and at least 2% water.

In one embodiment the phospholipid composition may comprise at least 5% water, or at least 10% water or at least 20% water.

In one aspect the phospholipid composition may comprise at most 30% water, or at most 40% water or at most 50% water.

As well as phospholipid and water, the phospholipid composition may comprise one or more further constituents such as triglyceride(s) or free fatty acids for example.

The term “plant phospholipid” as used herein means a phospholipid obtained or obtainable from a plant. Suitably the plant phospholipid may be one or more of phospholipids selected from the following group: phosphatidylcholine, phosphatidylethanolamine, phosphatidylinositol, phosphatidylserine and phosphatidylglycerol.

The phospholipid composition may be prepared by admixing the components thereof.

Suitably the phospholipid composition may comprise plant phospholipids from any plant or plant oil, such as from one or more of soya bean oil, canola oil, corn oil, cottonseed oil, palm oil, coconut oil, rice bran oil, peanut oil, olive oil, safflower oil, palm kernel oil, rape seed oil and sunflower oil.

Preferably, the plant phospholipids in the phospholipid composition are obtained or obtainable from one or more of soya bean oil, corn oil, sunflower oil and rape seed oil (sometimes referred to as canola oil).

More preferably, the plant phospholipids in the phospholipid composition is obtainable or obtained from one or more of soya bean oil, sunflower oil or rape seed oil.

Most preferably, the plant phospholipids in the phospholipid composition are obtainable or obtained from soya bean oil.

The present invention is particularly advantageous because it may utilise the by-products of plant processes as the starting materials.

For example, the phospholipid composition used in the present invention may be the by-product of degumming crude vegetable oil—in this process crude vegetable oil are degummed prior to or during refining to produce the degummed edible oil and a gum phase (the by-product). In this process crude oil is degummed (by for instance one or more of chemical degumming, enzymatic degumming, water degumming, total degumming and super degumming) to remove phosphatides, i.e. a mixture of polar lipids (in particular phospholipids) from the oil—the gum phase is thus a mixture of polar lipids, particularly phospholipids (together with other constituents such as water, triglycerides and free fatty acids for example). The water content in a gum composition (or gum phase) may be in the range of 10-40% w/w. The phospholipid content in a gum composition (or gum phase) may be in the range of 10-70% w/w. Thus in one embodiment the phospholipid composition according to the present invention may be a “gum-phase” or a “gum composition” obtained or obtainable from the degumming of vegetable oil.

Alternatively or in addition thereto the phospholipid composition used in the present invention may be a different by-product of refining crude vegetable oil—namely the soapstock. Soapstock is the by-product obtained by treating a crude vegetable oil with an acid and/or an alkaline (such as sodium hydroxide). Typically the resultant mixture is centrifuged to isolate the edible oil and a soapstock. The soapstock is thus a mixture of polar lipids, particularly phospholipids (together with other constituents such as water, triglycerides and salts of free fatty acids for example). The water content in a soapstock may be in the range of 10-65% or 10-70% w/w. The phospholipid content of the soapstock may be in the range of 10-70%. Thus in one embodiment the phospholipid composition according to the present invention may be a soapstock obtained or obtainable from acid and/or alkaline treatment of vegetable oil.

When the phospholipid composition is a gum composition (i.e. a gum phase) or a soapstock suitably the gum composition or soapstock may be purified, or dried, or solvent fractionated, or a combination of two or more thereof prior to admixing same with the lipid acyltransferase and the phytosterol and/or phytostanol, and optionally water.

In some embodiments the phospholipid composition used herein is a dry composition comprising no or very little water. Such phospholipid compositions may encompass dried gum phase compositions or dried soapstock. In such embodiments water may be added to the reaction composition to ensure that the reaction composition comprises at least 2%, preferably at least 5%, preferably at least 10%, more preferably at least 20% water.

In other embodiments the phospholipid composition in itself (i.e. naturally) may comprise some water, for example it may comprise at least 2% water (preferably at least 5%, preferably at least 10%, more preferably at least 20% water). Such phospholipid compositions include gum phase and soapstock compositions which have not been dried. In such embodiments it may be unnecessary to add additional water to the reaction composition providing there is sufficient water in the phospholipid composition itself so that in the reaction composition there is at least 2% water. However, additional water may be added to the reaction composition to increase the water content of the reaction composition if needed. The reaction composition should comprise at least 2% water (preferably at least 5%, preferably at least 10%; more preferably at least 20% water).

Suitably the phospholipid composition is comprised of a composition containing plant phospholipid and the water before the phospholipid composition is admixed with the lipid acyltransferase and/or the phytosterol or phytostanol. In one embodiment, the water may be admixed with the phospholipid to form a phospholipid composition at the same time or after mixing the phospholipid with the enzyme and/or the phytosterol and/or phytostanol.

For the avoidance of doubt the phospholipid composition according to the present invention is not a crude oil, e.g. a crude vegetable oil (which typically has a water content of less than 0.2% and a phospholipid content of no greater than 3%); nor it is a refined edible oil (which typically has no—or very little, typically less than 100 ppm—phospholipid).

Suitably the phospholipid composition may be incubated (or admixed) with the lipid acyltransferase at about 30 to about 70° C., preferably at about 40 to about 60° C., preferably at about 40 to about 50° C., preferably at about 40 to about 45° C.

In another embodiment, suitably the process and/or use according to the present invention may be carried out at below about 60° C., preferably below about 65° C., preferably below about 70° C.

Suitably the temperature of the phospholipid composition and/or the reaction composition may be at the desired reaction temperature when the enzyme is admixed therewith.

The phospholipid composition and/or phytosterol and/or phytostanol and/or water may be heated and/or cooled to the desired temperature before and/or during enzyme addition. Therefore in one embodiment it is envisaged that a further step of the process according to the present invention may be the cooling and/or heating of the phospholipid composition and/or phytosterol and/or phytostanol and/or water.

Preferably the water content for the process according to the present invention or for the phospholipid composition or reaction composition may be at least about 2% w/w. In one embodiment preferably the water content for the reaction composition or phospholipid composition according to the present invention may be at least about 5% w/w, or at least about 10% w/w, or at least about 20% w/w.

In some embodiments the water content for the process according to the present invention or the phospholipid composition may be between about 2% w/w to about 60% w/w, such as between about 5% w/w and about 50% w/w.

Suitably the reaction time (i.e. the time period in which the admixture is held), preferably with agitation, is for a sufficient period of time to transfer at least one acyl group from a plant phospholipid to a phytosterol and/or phytostanol thereby providing one or more phytostanol esters and/or phytosterol esters.

Preferably the reaction time is effective to ensure that there is at least 5% transferase activity, preferably at least 10% transferase activity, preferably at least 15%, 20%, 25% 26%, 28%, 30%, 40% 50%, 60%, 75%, 85% or 95% transferase activity. The % transferase activity (i.e. the transferase activity as a percentage of the total enzymatic activity) may be determined by the protocol taught below.

The % conversion of the phytosterol in the present invention is at least 1%, preferably at least 5%, preferably at least 10%, preferably at least 20%, preferably at least 30%, preferably at least 40%, preferably at least 50%, preferably at least 60%, preferably at least 70%, preferably at least 80%, preferably at least 90%, preferably at least 95%.

Preferably the reaction time is for a sufficient period of time to esterify at least 50% of the phytosterols and/or phytostanols in the admixture or reaction composition, preferably at least 60%, more preferably at least 70%, more preferably at least 80%, even more preferably at least 90%. In some embodiments, preferably the reaction time is such that at least 95 or at least 98% of the phytosterols and/or phytostanols in the admixture or reaction composition are esterified.

In one embodiment the % conversion of the phytosterol in the present invention is at least 5%, preferably at least 20%, preferably at least 50%, preferably at least 80%, preferably at least 90%.

Suitably the reaction time (i.e. the time period in which the reaction composition or admixture is held), preferably with agitation, prior to isolating or purifying the phytosterol ester and/or phytostanol ester) may be between about 10 minutes to about 6 days, suitably between about 12 hours to about 5 days.

In some embodiments the reaction time may be between about 10 minutes and about 180 minutes, preferably between about 15 minutes and about 180 minutes, more preferably between about 15 minutes and 60 minutes, even more preferably between about 15 minutes and about 35 minutes, preferably between about 30 minutes and about 180 minutes, preferably between about 30 minutes and about 60 minutes.

In one embodiment preferably the reaction time may be between 1 day (24 hours) and 5 days. In one embodiment the process is preferably carried out at above about pH 4.5, above about pH 5 or above about pH 6.

Preferably the process is carried out between about pH 4.6 and about pH 10.0, more preferably between about pH 5.0 and about pH 10.0, more preferably between about pH 6.0 and about pH 10.0, more preferably between about pH 5.0 and about pH 7.0, more preferably between about pH 5.0 and about pH 6.5, and even more preferably between about pH 5.5 and pH 6.0.

In one embodiment the process may be carried out at a pH between about 5.3 and 8.3.

In one embodiment the process may be carried out at a pH between about 6-6.5, preferably about 6.3.

Suitably the pH may be neutral (about pH 5.0-about pH 7.0) in the methods and/or uses of the present invention.

In one embodiment the term “isolating” may mean the separating the phytosterol ester and/or phytostanol ester from at least some (preferably all) of at least one other component in the reaction admixture and/or reaction composition.

In one aspect the phytosterol ester and/or phytostanol ester may be isolated or separated from one or more of the other constituents of the reaction admixture or reaction composition. In this regard, the term “isolated” or “isolating” may mean that the phytosterol ester and/or phytostanol ester is at least substantially free from at least one other component found in the reaction admixture or reaction composition or is treated to render it at least substantially free from at least one other component found in the reaction admixture or reaction composition.

In one aspect the phytosterol ester and/or phytostanol ester is isolated or is in an isolated form.

In a further aspect the phytosterol ester and/or phytostanol ester may be purified or in a purified form.

In one aspect the term “purifying” means that the phytostanol ester and/or phytosterol ester is treated to render it in a relatively pure state—e.g. at least about 51% pure, or at least about 75%, or at least about 80%, or at least about 90% pure, or at least about 95% pure or at least about 98% pure.

The isolation or purification of the phytosterol ester and/or phytostanol ester from the other constituents of the admixture may be carried out by any conventional method. Preferably the isolation or purification is carried out by different unit operations, such as one or more of the following: extraction, pH adjustment, fractionation, washing, centrifugation and/or distillation.

In one embodiment the phospholipid composition, enzyme and phytosterol and/or phytostanol may be pumped in a stream simultaneously or substantially simultaneously through a mixer and into a holding tank.

Suitably the enzyme may be inactivated during and/or at the end of the process.

The enzyme may be inactivated before or after separation (or isolation or purification) of the phytosterol esters and/or phytostanol esters.

Suitably the enzyme may be heat deactivated by heating for 10 mins at 75-85° C. or at above 92° C.

Suitably the enzyme may be dosed in a range of about 0.01-100 TIPU-K/g phospholipid composition; suitably the enzyme may be dosed in the range of about 0.05 to 10 TIPU-K/g, preferably about 0.05 to 1.5 TIPU-K/g phospholipid composition, more preferably at 0.2-1 TIPU-K/g phospholipid composition.

The lipid acyltransferase suitably may be dosed in the range of about 0.01 TIPU-K units/g oil to 5 TIPU-K units/g phospholipid composition. In one embodiment the lipid acyltransferase may be dosed in the range of about 0.1 to about 1 TIPU-K units/g phospholipid composition, more preferably the lipid acyltransferase may be dosed in the range of about 0.1 to about 0.5 TIPU-K units/g phospholipid composition, more preferably the lipid acyltransferase may be dosed in the range of about 0.1 to about 0.3 TIPU-K units/g phospholipid composition.

### Phospholipase Activity, TIPU-K:

Substrate: 1.75% L-Plant Phosphatidylcholin 95% (441601, Avanti Polar Lipids), 6.3% Triton X-100 (#T9284, Sigma) and 5 mM CaCl₂ dissolved in 50 mm Hepes pH 7.0.

Assay procedure: Samples, calibration, and control were diluted in 10 mM HEPES pH 7.0, 0.1% Triton X-100 (#T9284, Sigma). Analysis was carried out using a Konelab Autoanalyzer (Thermo, Finland). The assay was run at 30 C. 34 μL substrate was thermostatted for 180 seconds, before 4 μL sample was added. Enzymation lasted 600 sec. The amount of free fatty acid liberated during enzymation was measured using the NEFA C kit (999-75406, WAKO, Germany). 113 μL NEFA A was added and the mixture was incubated for 300 sec. Afterwards, 56 μL NEFA B was added and the mixture was incubated for 300 sec. OD 520 nm was then measured. Enzyme activity (μmol FFA/mL) was calculated based on a standard enzyme preparation. Enzyme activity TIPU-K was calculated as micromole free fatty acid (FFA) produced per minute under assay conditions.

For the ease of reference, these and further aspects of the present invention are now discussed under appropriate section headings. However, the teachings under each section are not necessarily limited to each particular section.

### Advantages

The present invention provides a sustainable and environmentally friendly way to produce sterol esters and/or stanol esters.

One advantage of the present invention is that the reaction takes place at lower temperatures compared with conventional methods for producing sterol esters and/or stanol esters.

Another advantage of the present invention is that the reaction takes place in an aqueous system (i.e. a water based system). Therefore there is no need to use organic solvents in the process of the present invention. This is highly advantageous compared with conventional methods for producing sterol esters and/or stanol esters. In particular, the use of an aqueous system reduces the need for excessive purification and isolation (i.e. to remove all of the organic solvent) because often the admixture of the present invention itself has no constituents which would be considered unsuitable for use directly in a industrial composition, such as a food or feed composition or a personal care product (e.g. cosmetic) composition. Therefore the process of the present invention has the advantage that the sterol esters and stanol esters may be simply concentrated before use.

A further advantage of the present invention is that the process can utilise by-products of other plant processing—thus reducing waste and forming valuable sterol esters and/or stanol esters from lower value compositions. For instance, the phospholipid composition for use in the present invention may be a gum composition or soapstock (both of which are by-products of edible oil refining). In addition or as an alternative the phytosterol and/or phytostanol used in the present invention may be a soybean oil deodorizer distillate (SODD).

Another advantage is that the present invention allows for the production of sterol esters and stanol esters in high yields and in industrial amounts without the use of organic solvents during the enzymatic formation of the sterol esters and/or stanol esters.

A further advantage of the present invention is that the process for the production of sterol esters or stanol esters may be carried out at temperatures which are lower than temperatures used in conventional production processes for sterol esters or stanol esters. An advantage is therefore that the sterols, sterol esters, stanols or stanol esters are exposed to less oxidative stress compared with the sterols, stanols, sterol esters or stanol esters produced in conventional processes. One advantage therefore is that the sterol esters and/or stanol esters produced in accordance with the present invention are produced with fewer by-products being produced, e.g. from thermal and oxidative degradation of sterols, sterol esters, stanols or stanol esters compared with a chemical catalysed reaction. This results in simpler purification and isolation processes.

### Lipid Acyl Transferase

Any lipid acyltransferase may be used in the present invention.

For instance, the lipid acyl transferase for use in the present invention may be one as described in WO2004/064537, WO2004/064987, WO2005/066347, WO2006/008508 or WO2008/090395. These documents are incorporated herein by reference.

The lipid acyl transferase for use in any one of the methods and/or uses of the present invention may be a natural lipid acyl transferase or a variant lipid acyl transferase.

The term “lipid acyl transferase” as used herein preferably means an enzyme that has acyltransferase activity (generally classified as E.C. 2.3.1.x, for example 2.3.1.43), whereby the enzyme is capable of transferring an acyl group from a lipid to a sterol and/or a stanol, preferably a phytosterol and/or a phytostanol, as an acyl acceptor molecule.

Suitably the lipid acyltransferase is one classified under the Enzyme Nomenclature classification (E.C. 2.3.1.43).

Preferably, the lipid acyl transferase for use in any one of the methods and/or uses of the present invention is a lipid acyltransferase that is capable of transferring an acyl group from a phospholipid (as defined herein) to a phytosterol and/or a phytostanol.

Preferably, the “acyl acceptor” according to the present invention is not water.

Suitably, some of the acyl acceptor may be naturally found in the phospholipid composition. Alternatively (and preferably) the acyl acceptor may be added to the phospholipid composition (e.g. the acyl acceptor may be extraneous or exogenous to the phospholipid composition). This is particularly important if the amount of acyl acceptor is rate limiting on the acyltransferase reaction.

Preferably, the lipid substrate upon which the lipid acyltransferase acts is one or more of the following lipids: a phospholipid, such as a lecithin, e.g. phosphatidylcholine and/or phophatidylethanolamine.

This lipid substrate may be referred to herein as the “lipid acyl donor”. The term lecithin as used herein encompasses phosphatidylcholine, phosphatidylethanolamine, phosphatidylinositol, phosphatidylserine and phosphatidylglycerol.

Preferred lipid acyltransferases for use in the present invention are identified as those which have a high activity such as high phospholipid transferase activity on phospholipids in an aqueous environment; most preferably lipid acyl transferases for use in the present invention have a high phospholipid to phytosterol and/or phytostanol transferase activity.

Enzymes suitable for use in the methods and/or uses of the invention may have lipid acyltransferase activity as determined using the “Transferase Assay (sterol:phospholipid) (TrU)” below.

### Determination of Transferase Activity

### “Transferase Assay (Sterol:Phospholipid)” (TrU)

Substrate: 50 mg beta-sitosterol (Sigma S5753) and 450 mg Soya phosphatidylcholine(PC), Avanti #441601 is dissolved in chloroform, and chloroform is evaporated at 40° C. under vacuum.

300 mg PC: beta-sitosterol 9:1 is dispersed at 40° C. in 10 ml 50 mM HEPES buffer pH 7.

Enzymation:
250 μl substrate is added in a glass with lid at 40° C.
25 μl enzyme solution is added and incubated during agitation for 10 minutes at 40° C.

The enzyme added should esterify 2-5% of the beta-sitosterol in the assay.

Also a blank with 25 μl water instead of enzyme solution is analysed.

After 10 minutes 5 ml Hexan:Isopropanol 3:2 is added.

The amount of beta-sitosterol ester is analysed by HPTLC using Cholesteryl stearate (Sigma C3549) standard for calibration.

Transferase activity is calculated as the amount of beta-sitosterol ester formation per minute under assay conditions.

One Transferase Unit (TrU) is defined as umol beta-sitosterol ester produced per minute at 40° C. and pH 7 in accordance with the transferase assay given above.

Preferably, the lipid acyltransferase used in the method and uses of the present invention will have a specific transferase unit (TrU) per mg enzyme of at least 25 TrU/mg enzyme protein.

Suitably the lipid acyltransferase for use in the present invention may be dosed in amount of 0.05 to 50 TrU per g phospholipid composition, suitably in an amount of 0.5 to 5 TrU per g phospholipid composition.

More preferably the enzymes suitable for use in the methods and/or uses of the present invention have lipid acyl-transferase activity as defined by the protocol below:

### Protocol for the Determination of % Acyltransferase Activity:

A phospholipid composition to which a lipid acyltransferase (and a certain amount of sterol/stanol) according to the present invention has been added may be extracted following the enzymatic reaction with CHCl₃:CH₃OH 2:1 and the organic phase containing the lipid material is isolated and analysed by GLC and HPLC according to the procedure detailed hereinbelow. From the GLC and HPLC analyses the amount of free fatty acids and one or more of sterol/stanol esters; are determined. A control phospholipid composition to which no enzyme according to the present invention has been added, is analysed in the same way.
Calculation: From the results of the GLC and HPLC analyses the increase in free fatty acids and sterol/stanol esters can be calculated:

Δ% fatty acid=% Fatty acid(enzyme)−% fatty acid(control);

My fatty acid=average molecular weight of the fatty acids;

A=Δ% sterol ester/Mv sterol ester (where Δ% sterol ester=% sterol/stanol ester(enzyme)−% sterol/stanol ester(control) and My sterol ester=average molecular weight of the sterol/stanol esters);

The transferase activity is calculated as a percentage of the total enzymatic activity:

$$\% transferase activity = \frac{A \times 100}{A + Δ \% fatty acid \text{/} ( Mv fatty acid )}$$

For the assay the enzyme dosage used is preferably 0.2 TIPU-K/g phospholipid composition, more preferably 0.08 TIPU-K/g phospholipid composition, preferably 0.01 TIPU-K/g oil. The level of phospholipid present in the phospholipid composition and/or the % conversion of sterol is preferably determined after 0.5, 1, 2, 4 and 20 hours, more preferably after 20 hours.

Preferably the lipid acyltransferases for use in the present invention have a transferase activity of at least 15%, preferably at least 20%, preferably at least 30%, more preferably at least 40% when tested using the “Protocol for the determination of % acyltransferase activity”.

In addition to, or instead of, assessing the % transferase activity in a phospholipid composition (above), to identify the lipid acyl transferase enzymes most preferable for use in the methods of the invention the following assay entitled “Protocol for identifying lipid acyltransferases” can be employed.

### Protocol for Identifying Lipid Acyltransferases

A lipid acyltransferase in accordance with the present invention is one which results in:
i) the removal of phospholipid present in a soya bean oil supplemented with plant sterol (1%), water (1%) and phosphatidylcholine (2%) oil (using the method: Plant sterol, water and phosphatidylcholine were dissolved in soya bean oil by heating to 95° C. during agitation. The oil was then cooled to 40° C. and the enzymes were added. The sample was maintained at 40° C. with magnetic stirring and samples were taken out after 0.5, 1, 2, 4 and 20 hours and analysed by TLC); and/or
ii) the conversion (% conversion) of the added sterol to sterol-ester (using the method taught in i) above).

For the assay the enzyme dosage used may be 0.2 TIPU-K/g oil, preferably 0.08 TIPU-K/g oil, preferably 0.01 TIPU-K/g oil. The level of phospholipid present in the oil and/or the conversion (% conversion) of sterol is preferably determined after 0.5, 1, 2, 4 and 20 hours, more preferably after 20 hours.

In some aspects, the lipid acyltransferase for use in any one of the methods and/or uses of the present invention may comprise a GDSX motif and/or a GANDY motif.

Preferably, the lipid acyltransferase enzyme is characterised as an enzyme which possesses acyltransferase activity and which comprises the amino acid sequence motif GDSX, wherein X is one or more of the following amino acid residues L, A, V, I, F, Y, H, Q, T, N, M or S.

Suitably, the nucleotide sequence encoding a lipid acyltransferase or lipid acyltransferase for use in any one of the methods and/or uses of the present invention may be obtainable, preferably obtained, from an organism from one or more of the following genera: Aeromonas, Streptomyces, Saccharomyces, Lactococcus, Mycobacterium, Streptococcus, Lactobacillus, Desulfitobacterium, Bacillus, Campylobacter, Vibrionaceae, Xylella, Sulfolobus, Aspergillus, Schizosaccharomyces, Listeria, Neisseria, Mesorhizobium, Ralstonia, Xanthomonas and Candida. Preferably, the lipid acyltransferase is obtainable, preferably obtained, from an organism from the genus Aeromonas.

In one aspect of the present invention the lipid acyltransferase is a polypeptide having lipid acyltransferase activity which polypeptide is obtainable by expression of:
a) a nucleotide sequence comprising the nucleotide sequence shown as SEQ ID No. 49 or a nucleotide sequence which as has 75% or more identity (preferably at least 80%, more preferably at least 90% identical) therewith;
b) a nucleic acid which encodes said polypeptide wherein said polypeptide is at least 70% (preferably at least 80%, more preferably at least 90% identical) identical with the polypeptide sequence shown in SEQ ID No. 16 or with the polypeptide sequence shown in SEQ ID No. 68;
c) a nucleic acid which hybridises under medium (or high) stringency conditions to a nucleic probe comprising the nucleotide sequence shown as SEQ ID No. 49; or
d) a nucleic acid which is a fragment of the nucleic acid sequences specified in a), b) or c).

In one embodiment preferably the lipid acyltransferase for use in the present invention is a polypeptide obtainable by expression of a nucleotide sequence, particularly the nucleotide sequence shown herein as SEQ ID No. 49, in Bacillus licheniformis.

In one aspect preferably the lipid acyltransferase for use in the present invention is a polypeptide having lipid acyltransferase activity which polypeptide comprises any one of the amino acid sequences shown as SEQ ID No. 68, SEQ ID No. 16, SEQ ID No. 1, SEQ ID No. 3, SEQ ID No. 4, SEQ ID No. 5, SEQ ID No. 6, SEQ ID No. 7, SEQ ID No. 8, SEQ ID No. 9, SEQ ID No. 10, SEQ ID No. 11, SEQ ID No. 12, SEQ ID No. 13, SEQ ID No. 14, SEQ ID No. 15, SEQ ID No. 17, SEQ ID No. 18, SEQ ID No. 19, SEQ ID No. 34, SEQ ID No. 35 or an amino acid sequence which as has 75% or more identity therewith.

In a preferred aspect preferably the lipid acyltransferase for use in the present invention is a polypeptide having lipid acyltransferase activity which polypeptide comprises the amino acid sequence shown as SEQ ID No. 68 or SEQ ID No. 16 or comprises an amino acid sequence which as has at least 75% identity therewith, preferably at least 80%, preferably at least 85%, preferably at least 95%, preferably at least 98% identity therewith.

In one embodiment the lipid acyltransferase for use in any one of the methods and/or uses of the present invention is encoded by a nucleotide sequence shown in SEQ ID No. 49, or is encoded by a nucleotide sequence which has at least 75% identity therewith, preferably at least 80%, preferably at least 85%, preferably at least 95%, preferably at least 98% identity therewith.

In addition or in the alternative, the nucleotide sequence encoding a lipid acyltransferase for use in any one of the methods and/or uses of the present invention encodes a lipid acyltransferase that may comprise the amino acid sequence shown as SEQ ID No. 68, or an amino acid sequence which has 75% or more homology thereto. Suitably, the nucleotide sequence encoding a lipid acyltransferase encodes a lipid acyltransferase that may comprise the amino acid sequence shown as SEQ ID No. 68.

In one embodiment preferably the lipid acyltransferase for use in any one of the methods and/or uses of the present invention is a lipid acyltransferase that is expressed in Bacillus licheniformis by transforming said B. licheniformis with a nucleotide sequence shown in SEQ ID No. 49 or a nucleotide sequence having at least 75% therewith (more preferably at least 80%, more preferably at least 85%, more preferably at least 95%, more preferably at least 98% identity therewith); culturing said B. licheniformis and isolating the lipid acyltransferase(s) produced therein.

In some aspects of the present invention, the nucleotide sequence encoding a lipid acyltransferase for use in any one of the methods and/or uses of the present invention encodes a lipid acyltransferase that comprises an aspartic acid residue at a position corresponding to N-80 in the amino acid sequence of the Aeromonas salmonicida lipid acyltransferase shown as SEQ ID No. 35.

In some aspects of the present invention, the lipid acyltransferase for use in any one of the methods and/or uses of the present invention is a lipid acyltransferase that comprises an aspartic acid residue at a position corresponding to N-80 in the amino acid sequence of the Aeromonas salmonicida lipid acyltransferase shown as SEQ ID No. 35.

As detailed above, other acyl-transferases suitable for use in the methods of the invention may be identified by identifying the presence of the GDSX, GANDY and HPT blocks either by alignment of the pFam00657 consensus sequence (SEQ ID No 2), and/or alignment to a GDSX acyltransferase, for example SEQ ID No 16. In order to assess their suitability for the present invention, i.e. identify those enzymes which have a transferase activity of at least 5%, more preferably at least 10%, more preferably at least 20%, more preferably at least 30%, more preferably at least 40%, more preferably 50%, more preferably at least 60%, more preferably at least 70%, more preferably at least 80%, more preferably at least 90% and more preferably at least 98% of the total enzyme activity, such acyltransferases are tested using the “Protocol for the determination of % acyltransferase activity” assay detailed hereinabove.

Preferably, the lipid acyltransferase enzyme may be characterised using the following criteria:
the enzyme possesses acyl transferase activity which may be defined as ester transfer activity whereby the acyl part of an original ester bond of a lipid acyl donor is transferred to an acyl acceptor to form a new ester; and
the enzyme comprises the amino acid sequence motif GDSX, wherein X is one or more of the following amino acid residues L, A, V, I, F, Y, H, Q, T, N, M or S.

Preferably, X of the GDSX motif is L or Y. More preferably, X of the GDSX motif is L. Thus, preferably the enzyme according to the present invention comprises the amino acid sequence motif GDSL.

The GDSX motif is comprised of four conserved amino acids. Preferably, the serine within the motif is a catalytic serine of the lipid acyl transferase enzyme. Suitably, the serine of the GDSX motif may be in a position corresponding to Ser-16 in Aeromonas hydrophila lipid acyltransferase enzyme taught in Brumlik & Buckley (Journal of Bacteriology April 1996, Vol. 178, No. 7, p 2060-2064).

To determine if a protein has the GDSX motif according to the present invention, the sequence is preferably compared with the hidden markov model profiles (HMM profiles) of the pfam database in accordance with the procedures taught in WO2004/064537 or WO2004/064987, incorporated herein by reference.

Preferably the lipid acyl transferase enzyme can be aligned using the Pfam00657 consensus sequence (for a full explanation see WO2004/064537 or WO2004/064987).

Preferably, a positive match with the hidden markov model profile (HMM profile) of the pfam00657 domain family indicates the presence of the GDSL or GDSX domain.

Preferably when aligned with the Pfam00657 consensus sequence the lipid acyltransferase for use in the methods or uses of the invention may have at least one, preferably more than one, preferably more than two, of the following, a GDSX block, a GANDY block, a HPT block. Suitably, the lipid acyltransferase may have a GDSX block and a GANDY block. Alternatively, the enzyme may have a GDSX block and a HPT block. Preferably the enzyme comprises at least a GDSX block. See WO2004/064537 or WO2004/064987 for further details.

Preferably, residues of the GANDY motif are selected from GANDY, GGNDA, GGNDL, most preferably GANDY.

The pfam00657 GDSX domain is a unique identifier which distinguishes proteins possessing this domain from other enzymes.

The pfam00657 consensus sequence is presented in FIG. 3 as SEQ ID No. 2. This is derived from the identification of the pfam family 00657, database version 6, which may also be referred to as pfam00657.6 herein.

The consensus sequence may be updated by using further releases of the pfam database (for example see WO2004/064537 or WO2004/064987).

In one embodiment, the lipid acyl transferase enzyme for use in any one of the methods and/or uses of the present invention is a lipid acyltransferase that may be characterised using the following criteria:
(i) the enzyme possesses acyl transferase activity which may be defined as ester transfer activity whereby the acyl part of an original ester bond of a lipid acyl donor is transferred to acyl acceptor to form a new ester;
(ii) the enzyme comprises the amino acid sequence motif GDSX, wherein X is one or more of the following amino acid residues L, A, V, I, F, Y, H, Q, T, N, M or S;
(iii) the enzyme comprises His-309 or comprises a histidine residue at a position corresponding to His-309 in the Aeromonas hydrophila lipid acyltransferase enzyme shown in FIGS. 2 and 4 (SEQ ID No. 1 or SEQ ID No. 3).

Preferably, the amino acid residue of the GDSX motif is L.

In SEQ ID No. 3 or SEQ ID No. 1 the first 18 amino acid residues form a signal sequence. His-309 of the full length sequence, that is the protein including the signal sequence, equates to His-291 of the mature part of the protein, i.e. the sequence without the signal sequence.

In one embodiment, the lipid acyl transferase enzyme for use any one of the methods and uses of the present invention is a lipid acyltransferase that comprises the following catalytic triad: Ser-34, Asp-306 and His-309 or comprises a serine residue, an aspartic acid residue and a histidine residue, respectively, at positions corresponding to Ser-34, Asp-306 and His-309 in the Aeromonas hydrophila lipid acyl transferase enzyme shown in FIG. 4 (SEQ ID No. 3) or FIG. 2 (SEQ ID No. 1). As stated above, in the sequence shown in SEQ ID No. 3 or SEQ ID No. 1 the first 18 amino acid residues form a signal sequence. Ser-34, Asp-306 and His-:309 of the full length sequence, that is the protein including the signal sequence, equate to Ser-16, Asp-288 and His-291 of the mature part of the protein, i.e. the sequence without the signal sequence. In the pfam00657 consensus sequence, as given in FIG. 3 (SEQ ID No. 2) the active site residues correspond to Ser-7, Asp-345 and His-348.

In one embodiment, the lipid acyl transferase enzyme for use any one of the methods and/or uses of the present invention is a lipid acyltransferase that may be characterised using the following criteria:
the enzyme possesses acyl transferase activity which may be defined as ester transfer activity whereby the acyl part of an original ester bond of a first lipid acyl donor is transferred to an acyl acceptor to form a new ester; and
the enzyme comprises at least Gly-32, Asp-33, Ser-34, Asp-134 and His-309 or comprises glycine, aspartic acid, serine, aspartic acid and histidine residues at positions corresponding to Gly-32, Asp-33, Ser-34, Asp-306 and His-309, respectively, in the Aeromonas hydrophila lipid acyltransferase enzyme shown in SEQ ID No. 3 or SEQ ID No. 1.

Suitably, the lipid acyltransferase enzyme for use in any one of the methods and/or uses of the present invention may be encoded by one of the following nucleotide sequences:
(a) the nucleotide sequence shown as SEQ ID No. 36;
(b) the nucleotide sequence shown as SEQ ID No. 38;
(c) the nucleotide sequence shown as SEQ ID No. 39;
(d) the nucleotide sequence shown as SEQ ID No. 42;
(e) the nucleotide sequence shown as SEQ ID No. 44;
(f) the nucleotide sequence shown as SEQ ID No. 46;
(g) the nucleotide sequence shown as SEQ ID No. 48;
(h) the nucleotide sequence shown as SEQ ID No. 49;
(i) the nucleotide sequence shown as SEQ ID No. 50;
(j) the nucleotide sequence shown as SEQ ID No. 51;
(k) the nucleotide sequence shown as SEQ ID No. 52;
(l) the nucleotide sequence shown as SEQ ID No. 53;
(m) the nucleotide sequence shown as SEQ ID No. 54;
(n) the nucleotide sequence shown as SEQ ID No. 55;
(o) the nucleotide sequence shown as SEQ ID No. 56;
(p) the nucleotide sequence shown as SEQ ID No. 57;
(q) the nucleotide sequence shown as SEQ ID No. 58;
(r) the nucleotide sequence shown as SEQ ID No. 59;
(s) the nucleotide sequence shown as SEQ ID No. 60;
the nucleotide sequence shown as SEQ ID No. 61;
(u) the nucleotide sequence shown as SEQ ID No. 62;
(v) the nucleotide sequence shown as SEQ ID No. 63;
(w) or a nucleotide sequence which has 70% or more, preferably 75% or more, identity with any one of the sequences shown as SEQ ID No. 36, SEQ ID No. 38, SEQ ID No. 39, SEQ ID No. 42, SEQ ID No. 44, SEQ ID No. 46, SEQ ID No. 48, SEQ ID No. 49, SEQ ID No. 50, SEQ ID No. 51, SEQ ID No. 52, SEQ ID No. 53, SEQ ID No. 54, SEQ ID No. 55, SEQ ID No. 56, SEQ ID No. 57, SEQ ID No. 58, SEQ ID No. 59, SEQ ID No. 60, SEQ ID No. 61, SEQ ID No. 62 or SEQ ID No. 63.

Suitably the nucleotide sequence may have 80% or more, preferably 85% or more, more preferably 90% or more and even more preferably 95% or more identity with any one of the sequences shown as SEQ ID No. 36, SEQ ID No. 38, SEQ ID No. 39, SEQ ID No. 42, SEQ ID No. 44, SEQ ID No. 46, SEQ ID No. 48, SEQ ID No. 49, SEQ ID No. 50, SEQ ID No. 51, SEQ ID No. 52, SEQ ID No. 53, SEQ ID No. 54, SEQ ID No. 55, SEQ ID No. 56, SEQ ID No. 57, SEQ ID No. 58, SEQ ID No. 59, SEQ ID No. 60, SEQ ID No. 61, SEQ ID No. 62 or SEQ ID No. 63.

Suitably, the lipid acyl transferase enzyme for use any one of the methods and/or uses of the present invention may be a lipid acyltransferase that comprises one or more of the following amino acid sequences:

(i) the amino acid sequence shown as SEQ ID No. 68

(ii) the amino acid sequence shown as SEQ ID No. 3

(iii) the amino acid sequence shown as SEQ ID No. 4

(iv) the amino acid sequence shown as SEQ ID No. 5

(v) the amino acid sequence shown as SEQ ID No. 6

(vi) the amino acid sequence shown as SEQ ID No. 7

(vii) the amino acid sequence shown as SEQ ID No. 8

(viii) the amino acid sequence shown as SEQ ID No. 9

(ix) the amino acid sequence shown as SEQ ID No. 10

(x) the amino acid sequence shown as SEQ ID No. 11

(xi) the amino acid sequence shown as SEQ ID No. 12

(xii) the amino acid sequence shown as SEQ ID No. 13

(xiii) the amino acid sequence shown as SEQ ID No. 14

(xiv) the amino acid sequence shown as SEQ ID No. 1

(xv) the amino acid sequence shown as SEQ ID No. 15

(xvi) the amino acid sequence shown as SEQ ID No. 16

(xvii) the amino acid sequence shown as SEQ ID No. 17

(xviii) the amino acid sequence shown as SEQ ID No. 18

(xix) the amino acid sequence shown as SEQ ID No. 34

(xx) the amino acid sequence shown as SEQ ID No. 35 or

an amino acid sequence which has 75%, 80%, 85%, 90%, 95%, 98% or more identity with any one of the sequences shown as SEQ ID No. 68, SEQ ID No. 1, SEQ ID No. 3, SEQ ID No. 4, SEQ ID No. 5, SEQ ID No. 6, SEQ ID No. 7, SEQ ID No. 8, SEQ ID No. 9, SEQ ID No. 10, SEQ ID No. 11, SEQ ID No. 12, SEQ ID No. 13, SEQ ID No. 14 or SEQ ID No. 15, SEQ ID No. 16, SEQ ID No. 17, SEQ ID No. 18, SEQ ID No. 34 or SEQ ID No. 35.

In one aspect, the lipid acyltransferase enzyme for use any one of the methods and/or uses of the present invention is a lipid acyltransferase that may be a lecithin:cholesterol acyltransferase (LCAT) or variant thereof (for example a variant made by molecular evolution).

Suitable LCATs are known in the art and may be obtainable from one or more of the following organisms for example: mammals, rat, mice, chickens, Drosophila melanogaster, plants, including Arabidopsis and Oryza sativa, nematodes, fungi and yeast.

A lipid acyltransferase enzyme for use in any one of the methods and/or uses of the present invention may be a lipid acyl transferases isolated from Aeromonas spp., preferably Aeromonas hydrophile or A. salmonicida, most preferably A. salmonicida or variants thereof.

It will be recognised by the skilled person that it is preferable that the signal peptides of the acyl transferase has been cleaved during expression of the transferase. The signal peptide of SEQ ID Nos. 1, 3, 4, 15 and 16 are amino acids 1-18. Therefore the most preferred regions are amino acids 19-335 for SEQ ID No. 1 and SEQ ID No. 3 (A. hydrophilia) and amino acids 19-336 for SEQ ID No. 4, SEQ ID No. 15 and SEQ ID No. 16. (A. salmonicida). When used to determine the homology of identity of the amino acid sequences, it is preferred that the alignments as herein described use the mature sequence.

Therefore the most preferred regions for determining homology (identity) are amino acids 19-335 for SEQ ID No. 1 and 3 (A. hydrophilia) and amino acids 19-336 for SEQ ID Nos. 4, 15 and 16 (A. salmonicida). SEQ ID Nos. 34 and 35 are mature protein sequences of a lipid acyl transferase from A. hydrophilia and A. salmonicida respectively which may or may not undergo further post-translational modification.

A lipid acyltransferase enzyme for use any one of the methods and uses of the present invention may be a lipid acyltransferase that may also be isolated from Thermobifida, preferably T. fusca, most preferably shown in SEQ ID Nos. 27, 28, 38, 40 or 47, or encoded by a nucleic acid comprising the nucleotide sequences SEQ ID No. 39 or 48.

A lipid acyltransferase enzyme for use any one of the methods and uses of the present invention may be a lipid acyltransferase that may also be isolated from Streptomyces, preferable S. avermitis, most preferably comprising SEQ ID No. 32. Other possible enzymes for use in the present invention from Streptomyces include those comprising the sequences shown as SEQ ID Nos. 5, 6, 9, 10, 11, 12, 13, 14, 26, 31, 33, 36, 37, 43 or 45 or encoded by the nucleotide sequences shown as SEQ ID No. 52, 53, 56, 57, 58, 59, 60 or 61.

An enzyme for use in the invention may also be isolated from Corynebacterium, preferably C. efficiens, most preferably comprising the sequences shown in SEQ ID No. 29 or SEQ ID No. 41, or encoded by the nucleotide sequences shown in SEQ ID No. 42.

In one embodiment the lipid acyltransferase according to the present invention may be a lipid acyltransferase obtainable, preferably obtained, from the Streptomyces strains L130 or L131 deposited by Danisco A/S of Langebrogade 1, DK-1001 Copenhagen K, Denmark under the Budapest Treaty on the International Recognition of the Deposit of Microorganisms for the purposes of Patent Procedure at the National Collection of Industrial, Marine and Food Bacteria (NCIMB) 23 St. Machar Street, Aberdeen Scotland, GB on 23 Jun. 2004 under accession numbers NCIMB 41226 and NCIMB 41227, respectively.

In one embodiment the enzyme according to the present invention may be preferably not be a phospholipase enzyme, such as a phospholipase A1 classified as E.C. 3.1.1.32 or a phospholipase A2 classified as E.C. 3.1.1.4.

### Variant Lipid Acyl Transferase

In a preferred embodiment the nucleotide sequence encoding a lipid acyltransferase for use in any one of the methods and/or uses of the present invention may encode a lipid acyltransferase that is a variant lipid acyl transferase.

Variants which have an increased activity on phospholipids, such as increased hydrolytic activity and/or increased transferase activity, preferably increased transferase activity on phospholipids may be used.

Preferably the variant lipid acyltransferase is prepared by one or more amino acid modifications of the lipid acyl transferases as defined hereinabove.

Suitably, the lipid acyltransferase for use in any one of the methods and uses of the present invention may be a lipid acyltransferase that may be a variant lipid acyltransferase, in which case the enzyme may be characterised in that the enzyme comprises the amino acid sequence motif GDSX, wherein X is one or more of the following amino acid residues L, A, V, I, F, Y, H, Q, T, N, M or S, and wherein the variant enzyme comprises one or more amino acid modifications compared with a parent sequence at any one or more of the amino acid residues defined in set 2 or set 4 or set 6 or set 7 (as defined in WO 2005/066347 and hereinbelow).

For instance the variant lipid acyltransferase may be characterised in that the enzyme comprises the amino acid sequence motif GDSX, wherein X is one or more of the following amino acid residues L, A, V, I, F, Y, H, Q, T, N, M or S, and wherein the variant enzyme comprises one or more amino acid modifications compared with a parent sequence at any one or more of the amino acid residues detailed in set 2 or set 4 or set 6 or set 7 (as defined in WO 2005/066347 and hereinbelow) identified by said parent sequence being structurally aligned with the structural model of P10480 defined herein, which is preferably obtained by structural alignment of P10480 crystal structure coordinates with 1IVN.PDB and/or 1DEO.PDB as defined in WO 2005/066347 and hereinbelow.

In a further embodiment a lipid acyltransferase for use in any one of the methods and/or uses of the present invention may be a variant lipid acyltransferase that may be characterised in that the enzyme comprises the amino acid sequence motif GDSX, wherein X is one or more of the following amino acid residues L, A, V, I, F, Y, H, Q, T, N, M or S, and wherein the variant enzyme comprises one or more amino acid modifications compared with a parent sequence at any one or more of the amino acid residues taught in set 2 identified when said parent sequence is aligned to the pfam consensus sequence (SEQ ID No. 2—FIG. 3) and modified according to a structural model of P10480 to ensure best fit overlap as defined in WO 2005/066347 and hereinbelow.

Suitably a lipid acyltransferase for use in any one of the methods and uses of the present invention may be a variant lipid acyltransferase enzyme that may comprise an amino acid sequence, which amino acid sequence is shown as SEQ ID No. 34, SEQ ID No. 3, SEQ ID No. 4, SEQ ID No. 5, SEQ ID No. 6, SEQ ID No. 7, SEQ ID No. 8, SEQ ID No. 9, SEQ ID No. 10, SEQ ID No. 11, SEQ ID No. 12, SEQ ID No. 13, SEQ ID No. 14, SEQ ID No. 1, SEQ ID No. 15, SEQ ID No. 25, SEQ ID No. 26, SEQ ID No. 27, SEQ ID No. 28, SEQ ID No. 29, SEQ ID No. 30, SEQ ID No. 32, SEQ ID No. 33 or SEQ ID No. 35 except for one or more amino acid modifications at any one or more of the amino acid residues defined in set 2 or set 4 or set 6 or set 7 (as defined in WO 2005/066347 and hereinbelow) identified by sequence alignment with SEQ ID No. 34.

Alternatively the lipid acyltransferase may be a variant lipid acyltransferase enzyme comprising an amino acid sequence, which amino acid sequence is shown as SEQ ID No. 34, SEQ ID No. 3, SEQ ID No. 4, SEQ ID No. 5, SEQ ID No. 6, SEQ ID No. 7, SEQ ID No. 8, SEQ ID No. 9, SEQ ID No. 10, SEQ ID No. 11, SEQ ID No. 12, SEQ ID No. 13, SEQ ID No. 14, SEQ ID No. 1, SEQ ID No. 15, SEQ ID No. 25, SEQ ID No. 26, SEQ ID No. 27, SEQ ID No. 28, SEQ ID No. 29, SEQ ID No. 30, SEQ ID No. 32, SEQ ID No. 33 or SEQ ID No. 35 except for one or more amino acid modifications at any one or more of the amino acid residues defined in set 2 or set 4 or set 6 or set 7 as defined in WO 2005/066347 and hereinbelow, identified by said parent sequence being structurally aligned with the structural model of P 10480 defined herein, which is preferably obtained by structural alignment of P10480 crystal structure coordinates with 1IVN.PDB and/or 1DEO.PDB as taught within WO 2005/066347 and hereinbelow.

Alternatively, the lipid acyltransferase may be a variant lipid acyltransferase enzyme comprising an amino acid sequence, which amino acid sequence is shown as SEQ ID No. 34, SEQ ID No. 3, SEQ ID No. 4, SEQ ID No. 5, SEQ ID No. 6, SEQ ID No. 7, SEQ ID No. 8, SEQ ID No. 9, SEQ ID No. 10, SEQ ID No. 11, SEQ ID No. 12, SEQ ID No. 13, SEQ ID No. 14, SEQ ID No. 1, SEQ ID No. 15, SEQ ID No. 25, SEQ ID No. 26, SEQ ID No. 27, SEQ ID No. 28, SEQ ID No. 29, SEQ ID No. 30, SEQ ID No. 32, SEQ ID No. 33 or SEQ ID No. 35 except for one or more amino acid modifications at any one or more of the amino acid residues taught in set 2 identified when said parent sequence is aligned to the pfam consensus sequence (SEQ ID No. 2) and modified according to a structural model of P10480 to ensure best fit overlap as taught within WO 2005/066347 and hereinbelow.

Preferably, the parent enzyme is an enzyme which comprises, or is homologous to, the amino acid sequence shown as SEQ ID No. 34 and/or SEQ ID No. 15 and/or SEQ ID No. 35.

Preferably, the lipid acyltransferase may be a variant enzyme which comprises an amino acid sequence, which amino acid sequence is shown as SEQ ID No. 34 or SEQ ID No. 35 except for one or more amino acid modifications at any one or more of the amino acid residues defined in set 2 or set 4 or set 6 or set 7 as defined in WO 2005/066347 and hereinbelow.

Other suitable variant lipid acyltransferases for use in the methods/uses of the present invention are those described in PCT/IB2009/054535.

The tertiary structure of the lipid acyltransferases has revealed an unusual and interesting structure which allows lipid acyltransferases to be engineered more successfully. In particular the lipid acyltransferase tertiary structure has revealed a cave and canyon structure the residues forming these structures are defined herein below.

Alterations in the cave region may (for example) alter the enzyme's substrate chain length specificity for example.

Alterations in the canyon (particularly some preferred key modifications) have been found to be important in for example enhancing or changing the enzyme's substrate specificity.

In particular it has been found by the present inventors that there are a number of modifications in the canyon which rank highly and produce interesting variants with improved properties—these can be found at positions 31, 27, 85, 86, 119 and 120. In some embodiments positions 31 and/or 27 are highly preferred.

These variant lipid acyltransferase enzyme may be encoded by a nucleotide sequence which has at least 90% identity with a nucleotide sequence encoding a parent lipid acyltransferase and comprise at least one modification (suitably at least two modifications) at a position(s) which corresponds in the encoded amino acid sequence to an amino acid(s) located in a) the canyon region of the enzyme and/or b) insertion site 1 and/or c) insertion site 2, wherein the canyon region, insertion site 1 and/or insertion site 2 of the enzyme is defined as that region which when aligned based on primary or tertiary structure corresponds to the canyon region, insertion site 1 or insertion site 2 of the enzyme shown herein as SEQ ID No. 16 or SEQ ID No. 68 as described herein below.

In one embodiment preferably the modification(s) at a position located in the canyon and/or insertion site 1 and/or insertion site 2 is combined with at least one modification at a position which corresponds in the encoded amino acid sequence to an amino acid located outside of the canyon region and/or insertion site 1 and/or insertion site 2.

In one embodiment, the lipid acyltransferase comprises at least one modification (suitably at least two modifications) at a position(s) which corresponds in the encoded amino acid sequence to an amino acid(s) located at position 27, 31, 85, 86, 122, 119, 120, 201, 245, 232, 235 and/or 236 (preferably at position 27, 31, 85, 86, 119 and/or 120, more preferably at position 27 and/or 31), wherein the position numbering is defined as that position which when aligned based on primary or tertiary structure corresponds to the same position of the enzyme shown herein as SEQ ID No. 16.

In a further embodiment, the variant lipid acyltransferase comprises at least one modification at a position(s) which corresponds in the encoded amino acid sequence to an amino acid(s) located at position 27 and/or 31 in combination with at least one further modification, wherein the position numbering is defined as that position which when aligned based on primary or tertiary structure corresponds to the same position of the enzyme shown herein as SEQ ID No. 16.

Suitably, the at least one further modification may be at one or more of the following positions 85, 86, 122, 119, 120, 201, 245, 23, 81, 82, 289, 227, 229, 233, 33, 207, 130, wherein the position numbering is defined as that position which when aligned based on primary or tertiary structure corresponds to the same position of the enzyme shown herein as SEQ ID No. 16.

The lipid acyltransferase amino acid sequence for use in the present invention may comprise a modified backbone such that at least one modification (suitably at least two modifications) is made at a position(s) which corresponds in the encoded amino acid sequence to an amino acid(s) located in a) the canyon region of the enzyme and/or b) insertion site 1 and/or c) insertion site 2, wherein the canyon region, insertion site 1 and/or insertion site 2 enzyme is defined as that region which when aligned based on primary or tertiary structure corresponds to the canyon region, insertion site 1 or insertion site 2, respectively, of the enzyme shown herein as SEQ ID No. 16 or SEQ ID No. 68.

In one embodiment preferably the modification(s) at a position located in the canyon and/or insertion site 1 and/or insertion site 2 is combined with at least one modification at a position which corresponds in the encoded amino acid sequence to an amino acid located outside of the canyon region and/or insertion site 1 and/or insertion site 2.

Preferably, the lipid acyltransferase amino acid sequence backbone is modified such that at least one modification (suitably at least two modifications) is made at a position(s) which corresponds in the encoded amino acid sequence to an amino acid(s) located in position 27, 31, 85, 86, 122, 119, 120, 201, 245, 232, 235 and/or 236 (preferably at position 27, 31, 85, 86 119 and/or 120, more preferably at position 27 and/or 31), wherein the position numbering is defined as that position which when aligned based on primary or tertiary structure corresponds to the same position of the enzyme shown herein as SEQ ID No. 16.

In further preferred embodiments, the lipid acyltransferase amino acid sequence backbone comprises at least one modification (suitably at least two modifications) at a position(s) which corresponds in the encoded amino acid sequence to an amino acid(s) located in position 27, 31 in combination with at least one further modification, wherein the position numbering is defined as that position which when aligned based on primary or tertiary structure corresponds to the same position of the enzyme shown herein as SEQ ID No. 16.

Suitably, the at least one further modification may be at one or more of the following positions 85, 86, 122, 119, 120, 201, 245, 23, 81, 82, 289, 227, 229, 233, 33, 207, 130, wherein the position numbering is defined as that position which when aligned based on primary or tertiary structure corresponds to the same position of the enzyme shown herein as SEQ ID No. 16.

Further provided is an altered or variant lipid acyltransferase for use in the present invention comprising an amino acid sequence that is at least 70% identical to the lipid acyltransferase from Aeromonas salmonicida shown herein as SEQ ID No. 16 or 68, wherein a substrate chain length specificity determining segment that lies immediately N-terminal of the Asp residue of the catalytic triad of said altered lipid acyltransferase has an altered length relative to said lipid acyltransferase from Aeromonas salmonicida shown herein as SEQ ID No. 16 or 68.

Preferably the alteration comprises an amino acid insertion or deletion in said substrate chain length specificity determining segment, such as substituting said substrate chain length specificity determining segment of said parent enzyme with the substrate chain length specificity determining segment of a different lipid acyltransferase to produce said altered lipid acyltransferase. Preferably, said altering increases the length of acyl chain that can be transferred by said lipid acyltransferase.

Preferably, the altered lipid acyltransferase comprises an amino acid sequence that is at least 90% identical to the lipid acyltransferase from Aeromonas salmonicida shown herein as SEQ ID No. 16 or 68.

The nucleotide sequence encoding the variant lipid acyltransferase enzyme before modification is a nucleotide sequence shown herein as SEQ ID No. 69, SEQ ID No. 49, SEQ ID No. 50, SEQ ID No. 51, SEQ ID No. 62, SEQ ID No. 63 or SEQ ID No. 24; or is a nucleotide sequence which has at least 70% identity (preferably at least 80%, more preferably at least 90%, even more preferably at least 95% identity) with a nucleotide sequence shown herein as SEQ ID No. 69, SEQ ID No. 49, SEQ ID No. 50, SEQ ID No. 51, SEQ ID No. 62, SEQ ID No, 63 or SEQ ID No. 24; or is a nucleotide sequence which is related to SEQ ID No. 69, SEQ ID No. 49, SEQ ID No. 50, SEQ ID No. 51, SEQ ID No. 62, SEQ ID No. 63, SEQ ID No. 24 by the degeneration of the genetic code; or is a nucleotide sequence which hybridises under medium stringency or high stringency conditions to a nucleotide sequence shown herein as SEQ ID No. 69, SEQ ID No. 49, SEQ ID No. 50, SEQ ID No. 51, SEQ ID No. 62, SEQ ID No. 63 or SEQ ID No. 24.

In a preferred embodiment, the variant lipid acyltransferase is encoded by a nucleic acid (preferably an isolated or recombinant nucleic acid) sequence which hybridises under medium or high stringency conditions over substantially the entire length of SEQ ID No. 49 or SEQ ID No. 69 or a compliment of SEQ ID No. 49 or SEQ ID No. 69, wherein the encoded polypeptide comprising one or more amino acid residues selected from Q, H, N, T, F, Y or C at position 31; R, Y, S, V, I, A, T, M, F, C or L at position 86; R, G, H, K, Y, D, N, V, C, Q, L, E, S or F at position 27; H, R, D, E 85; T or I at position 119; K or E at position 120; S, L, A, F, W, Y, R, H, M or C at position 122; R at position 201; S as position 245; A or V at position 235; G or S at position 232; G or E at position 236, wherein the positions are equivalent amino acid positions with respect of SEQ ID No. 16.

The variant lipid acyltransferase may comprise a pro-peptide or a polypeptide which has lipid acyltransferase activity and comprises an amino acid sequence which is at least 90% (preferably at least 95%, more preferably at least 98%, more preferably at least 99%) identical with the amino acid sequence shown as SEQ ID No. 16 or 68 and comprises one or more modifications at one or more of the following positions: 27, 31, 85, 86, 122, 119, 120, 201, 245, 232, 235 and/or 236 (preferably at position 27, 31, 85, 86, 119 and/or 120 more preferably at position 27 and/or 31).

In one embodiment the variant comprises a pro-peptide or a polypeptide which has lipid acyltransferase activity and comprises an amino acid sequence shown as SEQ ID No. 16 or 68 except for one or more modifications at one or more of the following positions: 27, 31, 85, 86, 122, 119, 120, 201, 245, 232, 235 and/or 236 (preferably at position 27, 31, 85, 86, 119 and/or 120 more preferably at position 27 and/or 31).

In another embodiment, the lipid acyltransferase comprises a pro-peptide or a polypeptide which has lipid acyltransferase activity and comprises an amino acid sequence which is at least 90% (preferably at least 95%, more preferably at least 98%, more preferably at least 99%) identical with the amino acid sequence shown as SEQ ID No. 16 or 68 and comprises one or more modifications at positions 27 and/or 31 in combination with at least one further modification, wherein the position numbering is defined as that position which when aligned based on primary or tertiary structure corresponds to the same position of the enzyme shown herein as SEQ ID No. 6.

Suitably, the at least one further modification may be at one or more of the following positions 85, 86, 122, 119, 120, 201, 245, 23, 81, 82, 289, 227, 229, 233, 33, 207, 130, wherein the position numbering is defined as that position which when aligned based on primary or tertiary structure corresponds to the same position of the enzyme shown herein as SEQ ID No. 16.

In a preferred embodiment, the lipid acyltransferase comprises a pro-peptide or a polypeptide which has lipid acyltransferase activity and comprises an amino acid sequence shown as SEQ ID No. 16 or 68 except for one or more modifications at one or more of the following positions: 27 and/or 31 in combination with at least one further modification.

Suitably, the at least one further modification may be at one or more of the following positions 85, 86, 122, 119, 120, 201, 245, 23, 81, 82, 289, 227, 229, 233, 33, 207 and/or 130, wherein the position numbering is defined as that position which when aligned based on primary or tertiary structure corresponds to the same position of the enzyme shown herein as SEQ ID No. 16.

The lipid acyltransferase may be a pro-peptide which undergoes further post-translational modification to a mature peptide, i.e. a polypeptide which has lipid acyltransferase activity. By way of example only SEQ ID No. 68 is the same as SEQ ID No. 16 except that SEQ ID No. 68 has undergone post-translational and/or post-transcriptional modification to remove some amino acids, more specifically 38 amino acids. Therefore the polypeptide shown herein as SEQ ID No. 16 could be considered in some circumstances (i.e. in some host cells) as a pro-peptide—which is further processed to a mature peptide by post-translational and/or post-transcriptional modification. The precise modifications, e.g. cleavage site(s), in respect of the post-translational and/or post-transcriptional modification may vary slightly depending on host species. In some host species there may be no post translational and/or post-transcriptional modification, hence the pro-peptide would then be equivalent to the mature peptide (i.e. a polypeptide which has lipid acyltransferase activity). Without wishing to be bound by theory, the cleavage site(s) may be shifted by a few residues (e.g. 1, 2 or 3 residues) in either direction compared with the cleavage site shown by reference to SEQ ID No. 68 compared with SEQ ID No.16. In other words, rather than cleavage at position 235-ATR to position 273 (RRSAS) for example, the cleavage may commence at residue 232, 233, 234, 235, 236, 237 or 238 for example. In addition or alternatively, the cleavage may end at residue 270, 271, 272, 273, 274, 275 or 276 for example. In addition or alternatively, the cleavage may result in the removal of about 38 amino acids, in some embodiments the cleavage may result in the removal of between 30-45 residues, such as 34-42 residues, such as 36-40 residues, preferably 38 residues.

In some embodiments, in order to establish homology to primary structure, the amino acid sequence of a lipid acyltransferase is directly compared to the lipid acyltransferase enzyme shown herein as SEQ ID No. 16 or 68 primary sequence and particularly to a set of residues known to be invariant in all or most lipid acyltransferases for which sequences are known. After aligning the conserved residues, allowing for necessary insertions and deletions in order to maintain alignment (i.e., avoiding the elimination of conserved residues through arbitrary deletion and insertion), the residues equivalent to particular amino acids in the primary sequence of SEQ ID No. 16 or 68 are defined. In preferred embodiments, alignment of conserved residues conserves 100% of such residues. However, alignment of greater than 75% or as little as 50% of conserved residues are also adequate to define equivalent residues. In preferred embodiments, conservation of the catalytic serine and histidine residues are maintained. Conserved residues are used to define the corresponding equivalent amino acid residues of the lipid acyltransferase shown in SEQ ID No. 16 or 68 in other lipid acyltransferases, such as from other Aeromonas species, as well as any other organisms.

In order to align a parent lipid acyltransferase with SEQ ID No. 16 or SEQ ID No. 68 (the reference sequence), sequence alignment such as pairwise alignment can be used (http://www.ebi.ac.uk/emboss/align/index.html). Thereby, the equivalent amino acids in alternative parental lipid acyltransferase polypeptides, which correspond to one or more of the amino acids defined with reference to SEQ ID No. 68 or SEQ ID No. 16 can be determined and modified. As the skilled person will readily appreciate, when using the emboss pairwise alignment, standard settings usually suffice. Corresponding residues can be identified using “needle” in order to make an alignment that covers the whole length of both sequences. However, it is also possible to find the best region of similarity between two sequences, using “water”.

Alternatively, particularly in instances where parent lipid acyltransferase shares low primary sequence homology with SEQ ID No. 16 or SEQ ID No. 68, the corresponding amino acids in alternative parent lipid acyltransferase which correspond to one or more of the amino acids defined with reference to SEQ ID No. 16 or SEQ ID No. 68 can be determined by structural alignment to the structural model of SEQ ID No. 68 or SEQ ID No. 16, preferably SEQ ID No. 68.

Thus, equivalent residues may be defined by determining homology at the level of tertiary structure for a lipid acyltransferase whose tertiary structure has been determined by X-ray crystallography. In this context, “equivalent residues” are defined as those for which the atomic coordinates of two or more of the main chain atoms of a particular amino acid residue of the lipid acyltransferase shown herein as SEQ ID No. 16 or 68 (N on N, CA on CA, C on C, and O on O) are within 0.13 nm and preferably 0.1 nm after alignment. Alignment is achieved after the best model has been oriented and positioned to give the maximum overlap of atomic coordinates of non-hydrogen protein atoms of the lipid acyltransferase in question to the lipid acyltransferase shown herein as SEQ ID No. 16 or 68. As known in the art, the best model is the crystallographic model giving the lowest R factor for experimental diffraction data at the highest resolution available. Equivalent residues which are functionally and/or structurally analogous to a specific residue of the lipid acyltransferase as shown herein as SEQ ID No. 16 or 68 are defined as those amino acids of the lipid acyltransferase that preferentially adopt a conformation such that they either alter, modify or modulate the protein structure, to effect changes in substrate specification, e.g. substrate binding and/or catalysis in a manner defined and attributed to a specific residue of the lipid acyltransferase shown herein as SEQ ID No. 16 or 68. Further, they are those residues of the lipid acyltransferase (in cases where a tertiary structure has been obtained by x-ray crystallography), which occupy an analogous position to the extent that although the main chain atoms of the given residue may not satisfy the criteria of equivalence on the basis of occupying a homologous position, the atomic coordinates of at least two of the side chain atoms of the residue lie with 0.13 nm of the corresponding side chain atoms of the lipid acyltransferase shown herein as SEQ ID No. 16 or 68.

The coordinates of the three dimensional structure of the lipid acyltransferase shown herein as SEQ ID No. 68 (which is a Aeromonas salmonicida lipid acyltransferase comprising an N80D mutation) are described in PCT/IB2009/054535 and find use in determining equivalent residues on the level of tertiary structure.

There is a large insertion in the acyltransferase of Aeromonas salmonicida between the last beta strand and the ASP—X-X_HIS motif when compared to structurally similar E. coli thioesterase. This insertion creates a large cavity (hereinafter referred to as the “cave” that binds the aliphatic chain of the acyl enzyme intermediate. Modulating the sequence and size of this region results in a smaller or larger “cave” or cavity for the aliphatic chain of the acyl enzyme intermediate, i.e., the acyl chain that is transferred by the enzyme. Thus the enzymes of this family may be engineered to preferentially transfer acyl chains of different lengths.

Four insertions are found in the Aeromonas salmonicida lipid acyltransferase relative to the E. coli thioesterase (PDB entry 1IVN) that link common secondary structural elements common to both structures.

The amino acids coordinates of these insertions in the lipid acyltransferase shown here as SEQ ID No. 68 are listed in the Table below:

TABLE
Insertions in lipid acyltransferase:

| Insertion | Residues |
|---|---|
| Insertion 1 | 22-36 |
| Insertion 2 | 74-88 |
| Insertion 3 | 162-168 |
| Insertion 4 | 213-281 |

As described in detail in PCT/IB2009/054535 in the lipid acyltransferase, there is a large surface for substrate to bind that can be divided into two areas that are separated by Ser 16 and His 291, where Ser 16 and His 291 along with Asp288 form the characteristic catalytic triad. These two areas can be characterized as being a deep channel or “canyon”—hereinafter referred to the “canyon”—leading into an enclosed cavity or “cave” running through the molecule.

The residues forming the canyon are listed in the Table below:

TABLE
CANYON residues:

| | |
|---|---|
| Insertion 1 | M23, M27, Y30, L31 |
| Segment 1 | F42, G67, G68 |
| Insertion 2 | D80, P81, K82, Q84, V85, I86 |
| Segment 2a | Y117, A119, Y120 |
| Insertion 4 | G229, Y230, V231 |

The residues forming the cave are listed in table below.

TABLE
CAVE residues:

| | |
|---|---|
| Segment 1 | D15, S16, L18 |
| Segment 2 | W111, A114, L115, L118 |
| Segment 3 | P156, D157, L158, Q160, N161 |
| Segment 4 | F206, A207, E208, M209, L210 |
| Segment 5 | M285, F286, V290, H291, P292 V295 |

Segments 3 and 4 precede insertions 3 and 4 respectively, and segment 5 immediately follows insertion 4. Insertions 4 and 5 also contribute to the over enclosure resulting in the cave, thus the cave is different to the canyon in that insertions 1 and 2 form the lining of the canyon while insertions 3 and 4 form the overlaying structure. Insertions 3 and insertion 4 cover the cave.

In one embodiment the lipid acyltransferase for use in the present invention may be altered by modifying the amino acid residues in one or more of the canyon, the cave, the insertion 1, the insertion 2, the insertion 3 or the insertion 4.

In one embodiment the lipid acyltransferase for use in the present invention may be altered by modifying the amino acid residues in one or more of the canyon, insertion 1 or insertion 2.

In one embodiment, the dimensions of the acyl chain binding cavity of a lipid acyltransferase may be altered by making changes to the amino acid residues that form the larger cave. This may be done by modulating the size the regions that link the common features of secondary structure as discussed above. In particular, the size of the cave may be altered by changing the amino acids in the region between the last (fifth) beta strand of the enzyme and the Asp-X-X-His motif that forms part of the catalytic triad.

The substrate chain length specificity determining segment of a lipid acyltransferase is a region of contiguous amino acids that lies between the β5 β-strand of the enzyme and the Asp residue of the catalytic triad of that enzyme (the Asp residue being part of the Asp-Xaa-Xaa-His motif).

The tertiary structures of the Aeromonas salmonicida lipid acyltransferase and the E. coli thioesterase (deposited as NCBI's Genbank database as accession number 1FVN_A; GID:33357066) each showing a signature three-layer alpha/beta/alpha structure, where the beta-sheets are composed of five parallel strands allow the substrate chain length specificity determining segments of each of the lipid acyltransferase enzymes to be determined.

The substrate chain length specificity determining segment of the Aeromonas salmonicida lipid acyltransferase lies immediately N-terminal to the Asp residue of the catalytic triad of the enzyme. However, the length of the substrate chain length specificity determining segment may vary according to the distance between the Asp residue and the β5 β-strand of the enzyme. For example, the substrate chain length specificity determining segments of the lipid acyltransferase are about 13 amino, 19 amino acids and about 70 amino acids in length, respectively. As such, depending on the lipid acyltransferase, a substrate chain length specificity determining segment may be in the range of 10 to 70 amino acids in length, e.g., in the range of 10 to 30 amino acids in length, 30 to 50 amino acids in length, or 50 to 70 amino acids.

The Table below provides an exemplary sequence for the substrate chain length specificity determining segment of the lipid acyltransferase enzyme.

A. salmonicida lipid acyltransferase (GCAT)

SEQ ID No. 73
AEMLRDPQNFGLSDVENPCYDGGYVWKPFATRSVSTDRQLSASPQERLA
IAGNPLLAQAVASPMARRSASPLNCEGKMF

In certain embodiments, the amino acid sequence of a substrate chain length specificity determining segment may or may not be the amino acid sequence of a wild-type enzyme. In certain embodiments, the substrate chain length specificity determining segment may have an amino acid sequence that is at least 70%, e.g., at least 80%, at least 90% or at least 95% identical to the substrate chain length specificity determining segment of a wild type lipid acyltransferase.

Suitably the variant enzyme may be prepared using site directed mutagenesis.

Preferred modifications are located at one or more of the following positions L031, 1086, MO27, V085, A119, Y120, W122, E201, F235, W232, A236, and/or Q245.

In particular key modifications include one or more of the following modifications: L31Q, H, N, T, F, Y or C (preferably L31 Q); M27R, G, H, K, Y, D, N, V, C, Q, L, E, S or F (preferably M27V); V85H, R, D or E; I86R, Y, S, V, I, A, T, M, F, C or L (preferably 186S or A); A119T or I; Y120K or E; W122S, L or A (preferably W122L); E201R; Q245S; F235A or V; W232G or S; and/or A236G or E.

In one embodiment when the at least one modification is made in the canyon the modification(s) are made at one or more of the following positions: 31, 27, 85, 86, 119, 120.

In particular key modifications in the canyon include one or more of the following modifications: L31Q, H, N, T, F, Y or C (preferably L31 Q); M27R, G, H, K, Y, D, N, V, C, Q, L, E, S or F (preferably M27V); V85H, R, D or E; I86R, Y, S, V, I, A, T, M, F, C or L (preferably I86S or A); A119T or I; Y120K or E, which may be in combination with one another and/or in combination with a further modification.

In one embodiment preferably when the modification is made in insertion site 1 the modifications are made at one or more positions 31 and/or 27. Suitably the modifications may be L31Q, H, N, T, F, Y or C (preferably L31 Q) and/or M27R, G, H, K, Y, D, N, V, C, Q, L, E, S or F (preferably M27V).

In one embodiment preferably when the modification is made in insertion site 2 the modifications are made at positions are 085, 086. Suitably the modifications may be V85H, R, D or E and/or 186R, Y, S, V, I, A, T, M, F, C or L.

In one embodiment preferably when the modification is made in insertion site 4 the modifications are made at position 245. Suitably the modification may be Q245S.

In one embodiment preferably the modification is made in at least insertion site 1.

In another embodiment preferably a modification is made in at least insertion site 1 in combination with a further modification in insertion site 2 and/or 4 and/or at one or more of the following positions 119, 120, 122, 201, 77, 130, 82, 120, 207, 167, 227, 215, 230, 289.

In a further embodiment preferably a modification is made in at least the canyon region in combination with a further modification in insertion site 4 and/or at one or more of the following positions 122, 201, 77, 130, 82, 120, 207, 167, 227, 215, 230, 289.

Preferred modifications are given for particular site:

R130R, V, Q, H, A, D, L, I, K, N, C, Y, G, S, F, T or M;

K82R, N, H, S, L, E, T, M or G;

G121S, R, G, E, K, D, N, V, Q or A;

Y74Y or W;

Y83 F or P;

I77T, M, H, Q, S, C, A, E, L, Y, F, R or V;

A207E;

Q167T, H, I, G, L or M;

D227L, C, S, E, F, V, I, T, Y, P, G, R, D, H or A;

N215G;

Y230A, G, V, R, I, T, S, N, H, E, D, Q, K; or

N289P.

In combination with one or more modifications at positions 31, 27, 85, 86, 119, 120, 122, 201, 245, 235, 232, and/or 236 (for example the modification may be one or more of the following: L31Q, H, N, T, F, Y or C (preferably L31 Q); M27R, G, H, K, Y, D, N, V, C, Q, L, E, S or F (preferably M27V); V85H, R, D or E; I86R, Y, 5, V, I, A, T, M, F, C or L (preferably I86S or A); A119T or I; Y120K or E; W122S, L or A (preferably W122L); E201R; Q245S; F235A or V; W232G or S; and/or A236G or E) suitably the variant lipid acyltransferase may be additionally modified at one or more of the following positions 130, 82, 121, 74, 83, 77, 207, 167, 227, 215, 230, 289 (for example the additional modification may be one or more of the following: R130R, V, Q, H, A, D, L, I, K, N, C, Y, G, S, F, T or M; K82R, N, H, S, L, E, T, M or G; G121S, R, G, E, K, D, N, V, Q or A; Y74Y or W; Y83 F or P; I77T, M, H, Q, S, C, A, E, L, Y, F, R or V; A207E; Q167T, H, I, G, L or M; D227L, C, S, E, F, V, I, T, Y, P, G, R, D, H or A; N215G; Y230A, G, V, R, I, T, S, N, H, E, D, Q, K; and/or N289P), preferably the variant lipid acyltransferase may be additionally modified at least one or more of the following positions: 130, 82, 77 or 227.

For the avoidance of doubt the lipid acyltransferase backbone when aligned (on a primary or tertiary basis) with the lipid acyltransferase enzyme shown herein as SEQ ID No. 16 preferably has D in position 80. We have therefore shown in many of the combinations taught herein N80D as a modification. If N80D is not mentioned as a suitable modification and the parent backbone does not comprise D in position 80, then an additional modification of N80D should be incorporated into the variant lipid acyltransferase to ensure that the variant comprises D in position 80.

When the backbone or parent lipid acyltransferase already contains the N80D modification, the other modifications can be expressed without referencing the N80D modification, i.e. L31Q, N80D, W122L could have been expressed as L31Q, W122L for example.

However, it is important to note that the N80D modification is a preferred modification and a backbone enzyme or parent enzyme is preferably used which already possesses amino acid D in position 80. If, however, a backbone is used which does not contain amino acid D in position (such as one more of the lipid acyltransferases shown here as SEQ ID No. 1, 3, 4, 15, 34, or 35 for instance) then preferably an additional modification of N80D is included.

Suitably, the substitution at position 31 identified by alignment of the parent sequence with SEQ ID No. 68 or SEQ ID No. 16 may be a substitution to an amino acid residue selected from the group consisting of: Q, H, Y and F, preferably Q.

Suitably, the variant polypeptide comprises one or more further modification(s) at any one or more of amino acid residue positions: 27, 77, 80, 82, 85, 85, 86, 121, 122, 130, 167, 207, 227, 230 and 289, which position is identified by alignment of the parent sequence with SEQ ID No. 68. Suitably, at least one of the one or more further modification(s) may be at amino acid residue position: 86, 122 or 130, which position is identified by alignment of the parent sequence with SEQ ID No. 68.

Suitably, the variant lipid acyltransferase comprises one or more of the following further substitutions: I86 (A, C, F, L, M, 5, T, V, R, I or Y); W122 (S, A, F, W, C, H, L, M, R or Y); R130A, C, D, G, H, I, K, L, M, N, Q, T, V, R, F or Y); or any combination thereof.

The variant lipid acyltransferase may comprise one of the following combinations of modifications (where the parent back bone already comprises amino acid D in position 80, the modification can be expressed without reference to N80D):

L31Q, N80D, 186S, W122F

L31Q, N80D, W122L

L31Q, N80D, 186V, W122L

L31Q, N80D, 1861, W122L

L31Q, N80D, 186S, R130R

L31Q, N80D, K82R, 186A

L31Q, N80D, 186S, W122W

L31Q, N80D, 186S, W122Y

M27V, L31Q, N80D

L31Q, N80D, 186A, W122L

L31Q, N80D, W122L

L31Q, N80D, 186S, G121S

L31Q, N80D, 186S

L31Q, N80D, K82R, 186S

L31Q, N80D, 186S, W122L, R130Y

L31Q, N80D, 186S, W122L, R130V

L31Q, N80D, 186S

L31Q, N80D, 186T, W122L

L31Q, N80D, 186S, W122L

L31Q, N80D, W122L, R130Q

L31Q, N80D, 186S, W122L, R130R

L31Q, N80D, 186S

L31Q, N80D, G121R

L31Q, N80D, 186A

M27C, L31Q, N80D

M27Q, L31Q, N80D

L31Q, N80D, G121S

L31Q, N80D, 186S, W122R

L31Q, N80D, R130Q

L31Q, N80D, 186S, W122H

L31Q, N80D, 186M, W122L

L31Q, N80D, R130N

L31Q, N80D, 186S, W122L

L31Q, N80D, K82N

L31Q, N80D, 186S, W122M

L31Q, N80D, W122L

L31Q, N80D, K82H

L31Q, N80D, R130H

L31Q, N80D, R130A

L31Q, N80D, G121S

L31Q, N80D, 186S, W122L, R130D

L31Q, N80D, 186M

L31Q, Y74Y, N80D

L31Q, N80D, R130L

L31Q, N80D, Y83F

L31Q, N80D, K₈₂S

L31Q, 177T, N80D

L31Q, N80D, 186S, W122L, R130I

L31Q, N80D, 186S, W122L

L31Q, N80D, 186F, W122L

M27N, L31Q, N80D

L31Q, N80D, Y83P

L31Q, N80D, R130K

L31Q, N80D, K82R, 186S, W122L

L31Q, N80D, K82L

L31Q, N80D, 186S, G121G

L31Q, N80D, 186A, R130Q

M27H, L31Q, N80D

L31Q, N80D, W122L, A207E

L31Q, N80D, W122L, R130L

L31Q, N80D, K82E

L31Q, N80D, G121E

L31Q, N80D, W122L, R130R

L31Q, 177M, N80D

L31Q, N80D, K82T

L31Q, N80D, W122L

L31Q, N80D, W122H

L31Q, N80D, Q167T

L31Q, 177H, N80D

L31Q, N80D, G121K

L31Q, 177Q, N80D

L31Q, N80D, W122L, R130N

L31Q, N80D, W122L

L31Q, N80D, G121D

L31Q, N80D, R130T

L31Q, N80D, R130T

L31Q, N80D, K82M

L31Q, N80D, Q167H

L31Q, N80D, 186T

L31Q, N80D, Q167I

L31Q, N80D, 186C

L31Q, N80D, Q167G

M27L, L31Q, N80D

L31Q, N80D, 186S, G121R

L31Q, 177S, N80D

L31Q, 177C, N80D

L31Q, N80D, G121N

L31Q, 177A, N80D

L31Q, N80D, R130M

L31Q, N80D, W122F

M27G, L31Q, N80D

L31Q, N80D, K82G

L31Q, N80D, 186S, W122L, R130K

L31Q, N80D, R130A

L31Q, N80D, 1861

L31Q, 177E, N80D

L31Q, N80D, D227L

L31Q, N80D, V85H, N215G

L31Q, N80D, 186A, W122L, R130N

L31Q, 177R, N80D

L31Q, N80D, 186F

L31Q, N80D, 186Y, W122L

M27K, L31Q, N80D

L31Q, N80D, D227C

L31Q, N80D, R130L

L31Q, N80D, 186C, W122L

L31Q, N80D, Q167L

L31Q, N80D, V85H

L31Q, N80D, Q167M

M27D, L31Q, N80D

L31Q, N80D, 186L

L31Q, N80D, Y230A

L31Q, N80D, W122R

L31Q, N80D, Y230G

L31Q, N80D, D227S

L31Q, N80D, W122L, A207E, N289P

L31Q, N80D, W122Y

L31Q, N80D, 186L, W122L

L31Q, N80D, K82R, 186S, G121S, R130Q

L31Q, Y74W, N80D

L31Q, N80D, R130F

L31Q, N80D, G121V

L31Q, N80D, W122L, R130M

L31Q, N80D, R130V

L31Q, N80D, Y230V

L31Q, N80D, N215G

L31Q, N80D, 186S, W122L, R130N

L31Q, N80D, Y230R

M27E, L31Q, N80D

L31Q, N80D, Y230I

L31Q, N80D, 186S, W122L, R130S

L31Q, N80D, K82R

L31Q, N80D, D227E

L31Q, N80D, K82R, 186A, G121S

L31Q, N80D, R130G

L31Q, 177V, N80D

L31Q, N80D, G121G

L31Q, N80D, Y230T

L31Q, N80D, K82R, 186S, R130N

L31Q, N80D, D227F

L31Q, N80D, 186A, G121R

L31Q, N80D, 186S, R130N

L31Q, N80D, W122C

L31Q, N80D, Y230S

L31Q, N80D, R130Y

L31Q, N80D, R130C

L31Q, 177L, N80D

A119T, N80D

A199A, N80D

G67A, N80D, V85H

wherein said positions are identified by alignment of the parent sequence with SEQ ID No. 68 or SEQ ID No. 16.

Suitably, the variant lipid acyltransferase may be identical to the parent lipid acyltransferase except for a modification at position 31 and, optionally, one or more further modification(s) at any one or more of amino acid residue positions: 27, 77, 80, 82, 85, 85, 86, 121, 122, 130, 167, 207, 227, 230 and 289, which position is identified by alignment of the parent sequence with SEQ ID No. 68 or SEQ ID No. 16.

Suitably, the variant lipid acyltransferase may be identical to the parent lipid acyltransferase except for a modification at position 31 and, optionally, one or more further modification(s) at any one or more of amino acid residue positions: 86, 122 or 130, which position is identified by alignment of the parent sequence with SEQ ID No. 68 or SEQ ID No. 16.

In one embodiment, where the parent sequence is SEQ ID No. 16 or SEQ ID No. 68 or where the parent sequence is encoded by SEQ ID No. 49 or SEQ ID No. 69, the variant polypeptide has any one of the modifications as detailed above, except for a modification at position 80. In this regard, SEQ ID No. 16, SEQ ID No. 68 or a polypeptide encoded by SEQ ID No. 49 or SEQ ID No. 69 will already have aspartic acid at position 80, when said positions are identified by alignment of the parent sequence with SEQ ID No. 16.

Suitably, the variant lipid acyltransferase or the variant lipid acyltransferase may have at least 75% identity to the parent lipid acyltransferase, suitably the variant lipid acyltransferase may have at least 75% or at least 80% or at least 85% or at least 90% or at least 95% or at least 98% identity to the parent lipid acyltransferase.

The present invention also relates to a variant polypeptide having lipid acyltransferase activity, wherein the variant comprises a modification at least position 31 compared to a parent lipid acyltransferase, wherein position 31 is identified by alignment with SEQ ID No. 68 or SEQ ID No. 16.

In one embodiment preferably the variant lipid acyltransferase has the following modifications and/or the following modifications are made in the methods of the present invention:
L31Q, N80D, W122L (which can be expressed as L31Q, W122L where the backbone enzyme already has D in position 80);
M27V, L31Q, N80D (which can be expressed as N27V, L31Q where the backbone enzyme already has D in position 80);
L31Q, N80D, K82R, 186A (which can be expressed as L31Q, K82R, 186A where the backbone enzyme already has D in position 80); and/or
L31Q, N80D, 186S, W122F (which can be expressed as L31Q, 186S, W122F where the backbone enzyme already has D in position 80).

### Improved Properties

The variant lipid acyltransferase for use in the present invention have at least one improved property compared with a parent (i.e. backbone) or unmodified lipid acyltransferase.

The term “improved property” as used herein may include a) an altered substrate specificity of the lipid acyltransferase, for instance and by way of example only i) an altered ability of the enzymes to use certain compounds as acceptors, for example an improved ability to utilise a carbohydrate as an acceptor molecule thus improving the enzymes ability to produce a carbohydrate ester) or ii) an altering ability to use saturated or unsaturated fatty acids as a substrate or iii) a changed specificity such that the variant lipid acyltransferase preferentially utilises the fatty acid from the Sn1 or Sn2 position of a lipid substrate or iv) an altered substrate chain length specificity of in the variant enzyme; b) altered kinetics of the enzyme; and/or c) lowered ability of the variant lipid acyltransferase to carry out a hydrolysis reaction whilst maintaining or enhancing the enzymes ability to carry out an acyl transferase reaction.

Other improved properties may be for example related to improvements and/or changes in pH and/or temperature stability, and/or detergent and/or oxidative stability. Indeed, it is contemplated that enzymes having various degrees of stability in one or more of these characteristics (pH, temperature, proteolytic stability, detergent stability, and/or oxidative stability) can be prepared in accordance with the present invention.

Characterization of wild-type (e.g. parent lipid acyltransferase) and mutant (e.g. variant lipid acyltransferase) proteins is accomplished via any means suitable and is preferably based on the assessment of properties of interest.

In some embodiments the variant enzyme, when compared with the parent enzyme, may have an increased transferase activity and either the same or less hydrolytic activity. In other words, suitably the variant enzyme may have a higher transferase activity to hydrolytic activity (e.g. transferase: hydrolysis activity) compared with the parent enzyme. Suitably, the variant enzyme may preferentially transfer an acyl group from a lipid (including phospholipid, galactolipid or triacylglycerol) to an acyl acceptor rather than simply hydrolysing the lipid.

Suitably, the lipid acyltransferase for use in the invention may be a variant with enhanced enzyme activity on polar lipids, preferably phospholipids and/or glycolipids; when compared to the parent enzyme. Preferably, such variants also have low or no activity on lyso-polar lipids. The enhanced activity on polar lipids, preferably phospholipids and/or glycolipids, may be the result of hydrolysis and/or transferase activity or a combination of both. Preferably the enhanced activity on polar lipids in the result of transferase activity.

Variant lipid acyltransferases for use in the invention may have decreased activity on triglycerides, and/or monoglycerides and/or diglycerides compared with the parent enzyme.

Suitably the variant enzyme may have no activity on triglycerides and/or monoglycerides and/or diglycerides.

### DEFINITION OF SETS

Amino Acid Set 1:
Amino acid set 1 (note that these are amino acids in 1IVN—FIG. 53 and FIG. 54)
Gly8, Asp9, Ser10, Leu11, Ser12, Tyr15, Gly44, Asp45, Thr46, Glu69, Leu70, Gly71, Gly72, Asn73, Asp74, Gly75, Leu76, Gln106, Ile107, Arg108, Leu109, Pro110, Tyr113, Phe121, Phe139, Phe140, Met141, Tyr145, Met151, Asp154, His157, Gly155, Ile156, Pro158

The highly conserved motifs, such as GDSx and catalytic residues, were deselected from set 1 (residues underlined). For the avoidance of doubt, set 1 defines the amino acid residues within 10 Å of the central carbon atom of a glycerol in the active site of the 1IVN model.

Amino Acid Set 2:
Amino acid set 2 (note that the numbering of the amino acids refers to the amino acids in the P10480 mature sequence)
Leu17, Lys22, Met23, Gly40, Asn80, Pro81, Lys82, Asn87, Asn88, Trp111, Val112, Ala114, Tyr117, Leu118, Pro156, Gly159, G1n160, Asn161, Pro162, Ser163, Ala164, Arg165, Ser166, Gln167, Lys168, Val169, Val170, Glu171, Ala172, Tyr179, His180, Asn181, Met209, Leu210, Arg211, Asn215, Lys284, Met285, Gln289 and Val290.

Selected residues in Set 1 compared with Set 2 are shown in Table 1.

TABLE 1

| | |
|---|---|
| IVN model | P10480 |

| | A. hyd homologue | | Mature sequence |
|---|---|---|---|
| IVN | PFAM | Structure | Residue Number |
| Gly8 | Gly32 | | |
| Asp9 | Asp33 | | |
| Ser10 | Ser34 | | |
| Leu11 | Leu35 | | Leu17 |
| Ser12 | Ser36 | | Ser18 |
| | | | Lys22 |
| | | | Met23 |
| Tyr15 | Gly58 | | Gly40 |
| Gly44 | Asn98 | | Asn80 |
| Asp45 | Pro99 | | Pro81 |
| Thr46 | Lys100 | | Lys82 |
| | | | Asn87 |
| | | | Asn88 |
| Glu69 | Trp129 | | Trp111 |
| Leu70 | Val130 | | Val112 |
| Gly71 | Gly131 | | |
| Gly72 | Ala132 | | Ala114 |
| Asn73 | Asn133 | | |
| Asp74 | Asp134 | | |
| Gly75 | Tyr135 | | Tyr117 |
| Leu76 | Leu136 | | Leu118 |
| Gln106 | | Pro174 | Pro156 |
| Ile107 | | Gly177 | Gly159 |
| Arg108 | | Gln178 | Gln160 |
| Leu109 | | Asn179 | Asn161 |
| Pro110 | | 180 to 190 | Pro162 |
| Tyr113 | | | Ser163 |
| | | | Ala164 |
| | | | Arg165 |
| | | | Ser166 |
| | | | Gln167 |
| | | | Lys168 |
| | | | Val169 |
| | | | Val170 |
| | | | Glu171 |
| | | | Ala172 |
| Phe121 | His198 | Tyr197 | Tyr179 |
| | | His198 | His180 |
| | | Asn199 | Asn181 |
| Phe139 | Met227 | | Met209 |
| Phe140 | Leu228 | | Leu210 |
| Met141 | Arg229 | | Arg211 |
| Tyr145 | Asn233 | | Asn215 |
| | | | Lys284 |
| Met151 | Met303 | | Met285 |
| Asp154 | Asp306 | | |
| Gly155 | Gln307 | | Gln289 |
| Ile156 | Val308 | | Val290 |
| His157 | His309 | | |
| Pro158 | Pro310 | | |

Amino Acid Set 3:
Amino acid set 3 is identical to set 2 but refers to the Aeromonas salmonicida (SEQ ID No. 35) coding sequence, i.e. the amino acid residue numbers are 18 higher in set 3 as this reflects the difference between the amino acid numbering in the mature protein (SEQ ID No. 35) compared with the protein including a signal sequence (SEQ ID No. 4).

The mature proteins of Aeromonas salmonicida GDSX (SEQ ID No. 35) and Aeromonas hydrophila GDSX (SEQ ID No. 34) differ in five amino acids. These are Thr3Ser, LYS182G1n, Glu309Ala, Thr310Asn, and Gly318—, where the salmonicida residue is listed first and the hydrophila residue is listed last. The hydrophila protein is only 317 amino acids long and lacks a residue in position 318. The Aeromonas salmonicida GDSX has considerably high activity on polar lipids such as galactolipid substrates than the Aeromonas hydrophila protein. Site scanning was performed on all five amino acid positions.

Amino Acid Set 4:

Amino acid set 4 is S3, Q182, E309, S310, and −318.

Amino Acid Set 5:

F13S, D15N, S18G, S18V, Y30F, D116N, D116E, D157 N, Y226F, D228N Y230F.

Amino Acid Set 6:
Amino acid set 6 is Ser3, Leu17, Lys22, Met23, Gly40, Asn80, Pro81, Lys82, Asn 87, Asn88, Trp111, Val112, Ala114, Tyr117, Leu118, Pro156, Gly159, Gln160, Asn161, Pro162, Ser163, Ala164, Arg165, Ser166, Gln167, Lys168, Val169, Val170, Glu171, Ala172, Tyr179, His180, Asn181, Gln182, Met209, Leu210, Arg211, Asn215, Lys284, Met285, Gln289, Val290, Glu309, Ser310, −318.

The numbering of the amino acids in set 6 refers to the amino acids residues in P10480 (SEQ ID No. 3)—corresponding amino acids in other sequence backbones can be determined by homology alignment and/or structural alignment to P10480 and/or 1IVN.

Amino Acid Set 7:
Amino acid set 7 is Ser3, Leu17, Lys22, Met23, Gly40, Asn80, Pro81, Lys82, Asn 87, Asn88, Trp111, Val112, Ala114, Tyr117, Leu118, Pro156, Gly159, Gln160, Asn161, Pro162, Ser163, Ala164, Arg165, Ser166, Gln167, Lys168, Val169, Val170, Glu171, Ala172, Tyr179, His180, Asn181, Gln182, Met209, Leu210, Arg211, Asn215, Lys284, Met285, Gln289, Val290, Glu309, Ser310, −318, Y30X (where X is selected from A, C, D, E, G, H, I, K, L, M, N, P, Q, R, S, T, V, or W), Y226X (where X is selected from A, C, D, E, G, H, I, K, L, M, N, P, Q, R, S, T, V, or W), Y230X (where X is selected from A, C, D, E, G, H, I, K, L, M, N, P, Q, R, S, T, V, or W), S18X (where X is selected from A, C, D, E, F, H, I, K, L, M, N, P, Q, R, T, W or Y), D157X (where X is selected from A, C, E, F, G, H, I, K, L, M, P, Q, R, S, T, V, W or Y).

The numbering of the amino acids in set 7 refers to the amino acids residues in P10480 (SEQ ID No. 3)—corresponding amino acids in other sequence backbones can be determined by homology alignment and/or structural alignment to P10480 and/or 1IVN).

Suitably, the variant enzyme comprises one or more of the following amino acid modifications compared with the parent enzyme:

S3E, A, G, K, M, Y, R, P, N, T or G

E309Q, R or A, preferably Q or R

−318Y, H, S or Y, preferably Y.

Preferably, X of the GDSX motif is L. Thus, preferably the parent enzyme comprises the amino acid motif GDSL.

Suitably, said first parent lipid acyltransferase may comprise any one of the following amino acid sequences: SEQ ID No. 34, SEQ ID No. 3, SEQ ID No. 4, SEQ ID No. 5, SEQ ID No. 6, SEQ ID No. 7, SEQ ID No. 8, SEQ ID No. 9, SEQ ID No. 10, SEQ ID No. 11, SEQ ID No. 12, SEQ ID No. 13, SEQ ID No. 14, SEQ ID No. 1, SEQ ID No. 15, SEQ ID No. 25, SEQ ID No. 26, SEQ ID No. 27, SEQ ID No. 28, SEQ ID No. 29, SEQ ID No. 30, SEQ ID No. 32, SEQ ID No. 33 or SEQ ID No. 35.

Suitably, said second related lipid acyltransferase may comprise any one of the following amino acid sequences: SEQ ID No. 3, SEQ ID No. 34, SEQ ID No. 4, SEQ ID No. 5, SEQ ID No. 6, SEQ ID No. 7, SEQ ID No. 8, SEQ ID No. 9, SEQ ID No. 10, SEQ ID No. 11, SEQ ID No. 12, SEQ ID No. 13, SEQ ID No. 14, SEQ ID No. 1, SEQ ID No. 15, SEQ ID No. 25, SEQ ID No. 26, SEQ ID No. 27, SEQ ID No. 28, SEQ ID No. 29, SEQ ID No. 30, SEQ ID No. 32, SEQ ID No. 33 or SEQ ID No. 35.

The variant enzyme must comprise at least one amino acid modification compared with the parent enzyme. In some embodiments, the variant enzyme may comprise at least 2, preferably at least 3, preferably at least 4, preferably at least 5, preferably at least 6, preferably at least 7, preferably at least 8, preferably at least 9, preferably at least 10 amino acid modifications compared with the parent enzyme.

When referring to specific amino acid residues herein the numbering is that obtained from alignment of the variant sequence with the reference sequence shown as SEQ ID No. 34 or SEQ ID No. 35.

In one aspect preferably the variant enzyme comprises one or more of the following amino acid substitutions:

S3A, C, D, E, F, G, H, I, K, L, M, N, P, Q, R, T,
V, W, or Y;
and/or
L17A, C, D, E, F, G, H, I, K, M, N, P, Q, R, S, T,
V, W, or Y;
and/or
S18A, C, D, E, F, H, I, K, L, M, N, P, Q, R, T, W,
or Y;
and/or
K22A, C, D, E, F, G, H, I, L, M, N, P, Q, R, S, T,
V, W, or Y;
and/or
M23A, C, D, E, F, G, H, I, K, L, N, P, Q, R, S, T,
V, W, or Y;
and/or
Y30A, C, D, E, G, H, I, K, L, M, N, P, Q, R, S, T,
V, or W;
and/or
G40A, C, D, E, F, H, I, K, L, M, N, P, Q, R, S, T,
V, W, or Y;
and/or
N80A, C, D, E, F, G, H, I, K, L, M, P, Q, R, S, T,
V, W, or Y;
and/or
P81A, C, D, E, F, G, H, I, K, L, M, N, Q, R, S, T,
V, W, or Y;
and/or
K82A, C, D, E, F, G, H, I, L, M, N, P, Q, R, S, T,
V, W, or Y;
and/or
N87A, C, D, E, F, G, H, I, K, L, M, P, Q, R, S, T,
V, W, or Y;
and/or
N88A, C, D, E, F, G, H, I, K, L, M, P, Q, R, S, T,
V, W, or Y;
and/or
W111A, C, D, E, F, G, H, I, K, L, M, N, P, Q, R,
S, T, V, W or Y;
and/or
V112A, C, D, E, F, G, H, I, K, L, M, N, P, Q, R,
S, T, W, or Y;
and/or
A114C, D, E, F, G, H, I, K, L, M, N, P, Q, R, S,
T, V, W, or Y;
and/or
Y117A, C, D, E, F, G, H, I, K, L, M, N, P, Q, R,
S, T, V, or W;
and/or
L118A, C, D, E, F, G, H, I, K, M, N, P, Q, R, S,
T, V, W, or Y;
and/or
P156A, C, D, E, F, G, H, I, K, L, M, N, Q, R, S,
T, V, W, or Y;
and/or
D157A, C, E, F, G, H, I, K, L, M, P, Q, R, S, T,
V, W, or Y;
and/or
G159A, C, D, E, F, H, I, K, L, M, N, P, Q, R, S,
T, V, W, or Y;
and/or
Q160A, C, D, E, F, G, H, I, K, L, M, N, P, R, S,
T, V, W, or Y;
and/or
N161A, C, D, E, F, G, H, I, K, L, M P, Q, R, S, T,
V, W, or Y;
and/or
P162A, C, D, E, F, G, H, I, K, L, M, N, Q, R, S,
T, V, W, or Y;
and/or
S163A, C, D, E, F, G, H, I, K, L, M, N, P, Q, R,
T, V, W, or Y;
and/or
A164C, D, E, F, G, H, I, K, L, M, N, P, Q, R, S,
T, V, W, or Y;
and/or
R165A, C, D, E, F, G, H, I, K, L, M, N, P, Q, S,
T, V, W, or Y;
and/or
S166A, C, D, E, F, G, H, I, K, L, M, N, P, Q, R,
T, V, W, or Y;
and/or
Q167A, C, D, E, F, G, H, I, K, L, M, N, P, R, S,
T, V, W, or Y;
and/or
K168A, C, D, E, F, G, H, I, L, M, N, P, Q, R, S,
T, V, W, or Y;
and/or
V169A, C, D, E, F, G, H, I, K, L, M, N, P, Q, R,
S, T, W, or Y;
and/or
V170A, C, D, E, F, G, H, I, K, L, M, N, P, Q, R,
S, T, W, or Y;
and/or
E171A, C, D, F, G, H, I, K, L, M, N, P, Q, R, S,
T, V, W, or Y;
and/or
A172C, D, E, F, G, H, I, K, L, M, N, P, Q, R, S,
T, V, W, or Y;
and/or
Y179A, C, D, E, F, G, H, I, K, L, M, N, P, Q, R,
S, T, V, or W;
and/or
H180A, C, D, E, F, G, I, K, L, M, P, Q, R, S, T,
V, W, or Y;
and/or
N181A, C, D, E, F, G, H, I, K, L, M, P, Q, R, S,
T, V, W, or Y;
and/or
Q182A, C, D, E, F, G, H, I, K, L, M, N, P, R, S,
T, V, W, or Y, preferably K;
and/or
M209A, C, D, E, F, G, H, I, K, L, N, P, Q, R, S,
T, V, W, or Y;
and/or
L210 A, C, D, E, F, G, H, I, K, M, N, P, Q, R, S,
T, V, W, or Y;
and/or
R211 A, C, D, E, F, G, H, I, K, L, M, N, P, Q, R,
S, T, V, W, or Y;
and/or
N215 A, C, D, E, F, G, H, I, K, L, M, N, P, Q, R,
S, T, V, W, or Y;
and/or
Y226A, C, D, E, G, H, I, K, L, M, N, P, Q, R, S,
T, V, or W;
and/or
Y230A, C, D, E, G, H, I, K, L, M, N, P, Q, R, S,
T, V or W;
and/or
K284A, C, D, E, F, G, H, I, L, M, N, P, Q, R, S,
T, V, W, or Y;
and/or
M285A, C, D, E, F, G, H, I, K, L, N, P, Q, R, S,
T, V, W, or Y;
and/or
Q289A, C, D, E, F, G, H, I, K, L, M, N, P, R, S,
T, V, W, or Y;
and/or
V290A, C, D, E, F, G, H, I, K, L, M, N, P, Q, R,
S, T, W, or Y;
and/or
E309A, C, D, F, G, H, I, K, L, M, N, P, Q, R, S,
T, V, W, or Y;
and/or
S310A, C, D, E, F, G, H, I, K, L, M, N, P, Q, R,
T, V, W, or Y.

In addition or alternatively thereto there may be one or more C-terminal extensions. Preferably the additional C-terminal extension is comprised of one or more aliphatic amino acids, preferably a non-polar amino acid, more preferably of I, L, V or G. Thus, the present invention further provides for a variant enzyme comprising one or more of the following C-terminal extensions: 318I, 318L, 318V, 318G.

Preferred variant enzymes may have a decreased hydrolytic activity against a phospholipid, such as phosphatidylcholine (PC), may also have an increased transferase activity from a phospholipid.

Preferred variant enzymes may have an increased transferase activity from a phospholipid, such as phosphatidylcholine (PC), these may also have an increased hydrolytic activity against a phospholipid.

Modification of one or more of the following residues may result in a variant enzyme having an increased absolute transferase activity against phospholipid:
S3, D157, S310, E309, Y179, N215, K22, Q289, M23, H180, M209, L210, R211, P81, V112, N80, L82, N88; N87

Specific preferred modifications which may provide a variant enzyme having an improved transferase activity from a phospholipid may be selected from one or more of the following:
S3A, C, D, E, F, G, H, I, K, L, M, N, P, Q, R, T, V, W or Y; preferably N, E, K, R, A, P or M, most preferably S3A
D157A, C, E, F, G, H, I, K, L, M, N, P, Q, R, S, T, V, W or Y; preferably D157S, R, E, N, G, T, V, Q, K or C
S310A, C, D, E, F, G, H, I, K, L, M, N, P, Q, R, T, V, W or Y; preferably S310′T
−318 E
E309A, C, D, E, F, G, H, I, K, L, M, N, P, Q, R, T, V, W or Y; preferably E309 R, E, L, R or A
Y179A, C, D, E, F, G, H, I, K, L, M, N, P, Q, R, S, T, V or W; preferably Y179 D, T, E, R, N, V, K, Q or S, more preferably E, R, N, V, K or Q
N215A, C, D, E, F, G, H, I, K, L, M, P, Q, R, S, T, V, W or Y; preferably N215 S, L, R or Y
K22A, C, D, E, F, G, H, I, L, M, N, P, Q, R, S, T, V, W or Y; preferably K22 E, R, C or A
Q289A, C, D, E, F, G, H, I, K, L, M, N, P, R, S, T, V, W or Y; preferably Q289 R, E, G, P or N
M23A, C, D, E, F, G, H, I, K, L N, P, Q, R, S, T, V, W or Y; preferably M23 K, Q, L, G, T or S
H180A, C, D, E, F, G, I, K, L, M, P, Q, R, S, T, V, W or Y; preferably H180 Q, R or K
M209 A, C, D, E, F, G, H, I, K, L, N, P, Q, R, S, T, V, W or Y; preferably M209 Q, S, R, A, N, Y, E, V or L
L210A, C, D, E, F, G, H, I, K, M, N, P, Q, R, S, T, V, W or Y; preferably L210 R, A, V, S, T, I, W or M
R211A, C, D, E, F, G, H, I, K, L, M, N, P, Q, S, T, V, W or Y; preferably R211T
P81A, C, D, E, F, G, H, I, K, L, M, N, Q, R, S, T, V, W or Y; preferably P81G
V112A, C, D, E, F, G, H, I, K, L, M, N, P, Q, R, S, T, W or Y; preferably V112C
N80A, C, D, E, F, G, H, I, K, L, M, Q, R, S, T, V, W or Y; preferably N80 R, G, N, D, P, T, E, V, A or G
L82A, C, D, E, F, G, H, I, M, N, P, Q, R, S, T, V, W or Y; preferably L82N, S or E
N88A, C, D, E, F, G, H, I, K, L, M, P, Q, R, S, T, V, W or Y; preferably N88C
N87A, C, D, E, F, G, H, I, K, L, M, P, Q, R, S, T, V, W or Y; preferably N87M or G

Preferred modification of one or more of the following residues results in a variant enzyme having an increased absolute transferase activity against phospholipid:

S3 N, R, A, G

M23 K, Q, L, G, T, S

H180 R

L82 G

Y179 E, R, N, V, K or Q

E309 R, S, L or A

One preferred modification is N80D. This is particularly the case when using the reference sequence SEQ ID No. 35 as the backbone. Thus, the reference sequence may be SEQ ID No. 16. This modification may be in combination with one or more further modifications. Therefore in a preferred embodiment of the present invention the nucleotide sequence encoding a lipid acyltransferase for use in any one of the methods and uses of the present invention may encode a lipid acyltransferase that comprises SEQ ID No. 35 or an amino acid sequence which has 75% or more, preferably 85% or more, more preferably 90% or more, even more preferably 95% or more, even more preferably 98% or more, or even more preferably 99% or more identity to SEQ ID No. 35.

As noted above, when referring to specific amino acid residues herein the numbering is that obtained from alignment of the variant sequence with the reference sequence shown as SEQ ID No. 34 or SEQ ID No. 35.

Much by preference, the nucleotide sequence encoding a lipid acyltransferase for use in any one of the methods and uses of the present invention may encode a lipid comprising the amino acid sequence shown as SEQ ID No. 16 or the amino acid sequence shown as SEQ ID No. 68, or an amino acid sequence which has 70% or more, preferably 75% or more, preferably 85% or more, more preferably 90% or more, even more preferably 95% or more, even more preferably 98% or more, or even more preferably 99% or more identity to SEQ ID No. 16 or SEQ ID No. 68. This enzyme may be considered a variant enzyme.

In a preferred embodiment, the variant enzyme comprises one of SEQ ID No. 70, SEQ ID No. 71 or SEQ ID No. 72.

The degree of identity is based on the number of sequence elements which are the same. The degree of identity in accordance with the present invention for amino acid sequences may be suitably determined by means of computer programs known in the art, such as Vector NTI 10 (Invitrogen Corp.). For pairwise alignment the score used is preferably BLOSUM62 with Gap opening penalty of 10.0 and Gap extension penalty of 0.1.

Suitably, the degree of identity with regard to an amino acid sequence is determined over at least 20 contiguous amino acids, preferably over at least 30 contiguous amino acids, preferably over at least 40 contiguous amino acids, preferably over at least 50 contiguous amino acids, preferably over at least 60 contiguous amino acids.

Suitably, the degree of identity with regard to an amino acid sequence may be determined over the whole sequence.

Suitably, the nucleotide sequence encoding a lipid acyltransferase or the lipid acyl transferase enzyme for use in the present invention may be obtainable, preferably obtained, from organisms from one or more of the following genera: Aeromonas, Streptomyces, Saccharomyces, Lactococcus, Mycobacterium, Streptococcus, Lactobacillus, Desulfitobacterium, Bacillus, Campylobacter, Vibrionaceae, Xylella, Sulfolobus, Aspergillus, Schizosaccharomyces, Listeria, Neisseria, Mesorhizobium, Ralstonia, Xanthomonas, Candida, Thermobifida and Corynebacterium.

Suitably, the nucleotide sequence encoding a lipid acyltransferase or the lipid acyl transferase enzyme for use in the present invention may be obtainable, preferably obtained, from one or more of the following organisms: Aeromonas hydrophila, Aeromonas salmonicida, Streptomyces coelicolor, Streptomyces rimosus, Mycobacterium, Streptococcus pyogenes, Lactococcus lactis, Streptococcus pyogenes, Streptococcus thermophilus, Streptomyces thermosacchari, Streptomyces avermitilis Lactobacillus helveticus, Desulfitobacterium dehalogenans, Bacillus sp, Campylobacter jejuni, Vibrionaceae, Xylella fastidiosa, Sulfolobus solfataricus, Saccharomyces cerevisiae, Aspergillus terreus, Schizosaccharomyces pombe, Listeria innocua, Listeria monocytogenes, Neisseria meningitidis, Mesorhizobium loti, Ralstonia solanacearum, Xanthomonas campestris, Xanthomonas axonopodis, Candida parapsilosis, Thermobifida fusca and Corynebacterium efficiens.

In one aspect, preferably the nucleotide sequence encoding a lipid acyltransferase for use in any one of the methods and/or uses of the present invention encodes a lipid acyl transferase enzyme according to the present invention is obtainable, preferably obtained or derived, from one or more of Aeromonas spp., Aeromonas hydrophila or Aeromonas salmonicida.

In one aspect, preferably the lipid acyltransferase for use in any one of the methods and/or uses of the present invention is a lipid acyl transferase enzyme obtainable, preferably obtained or derived, from one or more of Aeromonas spp., Aeromonas hydrophila or Aeromonas salmonicida.

Enzymes which function as lipid acyltransferases in accordance with the present invention can be routinely identified using the assay taught herein below:
The term “transferase” as used herein is interchangeable with the term “lipid acyltransferase”.

Suitably, the lipid acyltransferase as defined herein catalyses one or more of the following reactions: interesterification, transesterification, alcoholysis, hydrolysis.

The term “interesterification” refers to the enzymatic catalysed transfer of acyl groups between a lipid donor and lipid acceptor, wherein the lipid donor is not a free acyl group.

The term “transesterification” as used herein means the enzymatic catalysed transfer of an acyl group from a lipid donor (other than a free fatty acid) to an acyl acceptor (other than water).

As used herein, the term “alcoholysis” refers to the enzymatic cleavage of a covalent bond of an acid derivative by reaction with an alcohol ROH so that one of the products combines with the H of the alcohol and the other product combines with the OR group of the alcohol,

As used herein, the term “alcohol” refers to an alkyl compound containing a hydroxyl group.

As used herein, the term “hydrolysis” refers to the enzymatic catalysed transfer of an acyl group from a lipid to the OH group of a water molecule.

The term “without increasing or without substantially increasing the free fatty, acids” as used herein means that preferably the lipid acyl transferase according to the present invention has 100% transferase activity (i.e. transfers 100% of the acyl groups from an acyl donor onto the acyl acceptor, with no hydrolytic activity); however, the enzyme may transfer less than 100% of the acyl groups present in the lipid acyl donor to the acyl acceptor. In which case, preferably the acyltransferase activity accounts for at least 5%, more preferably at least 10%, more preferably at least 20%, more preferably at least 30%, more preferably at least 40%, more preferably 50%, more preferably at least 60%, more preferably at least 70%, more preferably at least 80%, more preferably at least 90% and more preferably at least 98% of the total enzyme activity. The % transferase activity (i.e. the transferase activity as a percentage of the total enzymatic activity) may be determined by the following the “Assay for Transferase Activity” given above.

In some aspects of the present invention, the term “without substantially increasing free fatty acids” as used herein means that the amount of free fatty acid in a edible oil treated with an lipid acyltransferase according to the present invention is less than the amount of free fatty acid produced in the edible oil when an enzyme other than a lipid acyltransferase according to the present invention had been used, such as for example as compared with the amount of free fatty acid produced when a conventional phospholipase enzyme, e.g. Lecitase Ultra™ (Novozymes A/S, Denmark), had been used.

### Combinations

The enzyme for use according to the present invention may be used with one or more other suitable enzymes. Thus, it is within the scope of the present invention that, in addition to the lipid acyl transferase enzyme for use in the invention, at least one further enzyme is present in the reaction composition. Such further enzymes include starch degrading enzymes such as endo- or exoamylases, pullulanases, debranching enzymes, hemicellulases including xylanases, cellulases, oxidoreductases, e.g. peroxidases, phenol oxidases, glucose oxidase, pyranose oxidase, sulfhydryl oxidase, or a carbohydrate oxidase such as one which oxidises maltose, for example hexose oxidase (HOX), lipases, phospholipases, glycolipases, galactolipases and proteases.

In one embodiment the lipid acyltransferase is present in combination with a lipase having one or more of the following lipase activities: glycolipase activity (E.C. 3.1.1.26, triacylglycerol lipase activity (E.C. 3.1.1.3), phospholipase A2 activity (E.C. 3.1.1.4) or phospholipase A1 activity (E.C. 3.1.1.32). Suitable, lipolytic enzymes are well known in the art and include by way of example the following lipolytic enzymes: LIPOPAN® F, LIPOPAN®XTRA and/or LECITASE® ULTRA (Novozymes A/S, Denmark), phospholipase A2 (e.g. phospholipase A2 from LIPOMOD™ 22L from Biocatalysts, LIPOMAX™ from Genencor), LIPOLASE® (Novozymes A/S, Denmark), YIELDMAX™ (Chr. Hansen, Denmark), PANAMORE™ (DSM), the lipases taught in WO 03/97835, EP 0 977 869 or EP 1 193 314.

The use of the lipid acyl transferase may also be in the presence of a phospholipase, such as phospholipase A1, phospholipase A2, phospholipase B, Phospholipase C and/or phospholipase D.

The use of the lipid acyl transferase and the one more other suitable enzymes may be performed sequentially or concurrently, e.g. the lipid acyl transferase treatment may occur prior to, concurrently with or subsequently to enzyme treatment with the one more other suitable enzymes.

In the case of sequential enzyme treatments, in some embodiments it may be advantageous to remove the first enzyme used, e.g. by heat deactivation or by use of an immobilised enzyme, prior to treatment with the second (and/or third etc.) enzyme.

It will be further understood that the presence of the additional enzyme may be as a result of deliberate addition of the enzyme, or alternatively, the additional enzyme may be present as a contaminant or at a residual level resulting from an earlier process to which the phospholipid composition has been exposed.

### Post-Transcription and Post-Translational Modifications

Suitably the lipid acyltransferase in accordance with the present invention may be encoded by any one of the nucleotide sequences taught herein.

Depending upon the host cell used post-transcriptional and/or post-translational modifications may be made. It is envisaged that the lipid acyltransferase for use in the present methods and/or uses encompasses lipid acyltransferases which have undergone post-transcriptional and/or post-translational modification.

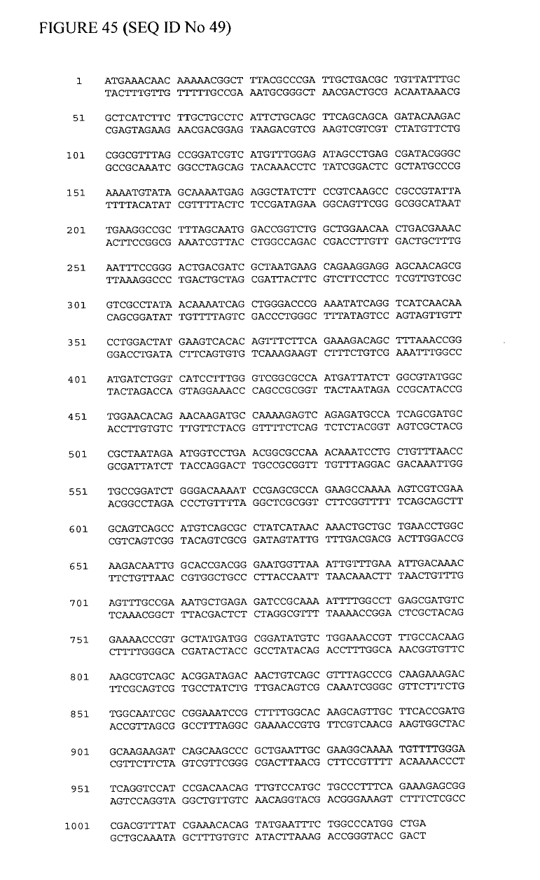

By way of example only, the expression of the nucleotide sequence shown herein as SEQ ID No. 49 (see FIG. 45) in a host cell (such as Bacillus licheniformis for example) results in post-transcriptional and/or post-translational modifications which leads to the amino acid sequence shown herein as SEQ ID No. 68.

SEQ ID No. 68 is the same as SEQ ID No. 16 except that SEQ ID No. 68 has undergone post-translational and/or post-transcriptional modification to remove some amino acids, more specifically 38 amino acids. Notably the N-terminal and C-terminal part of the molecule are covalently linked by an S-S bridge between two cysteines. Amino residues 236 and 236 of SEQ ID No. 38 are not covalently linked following post-translational modification. The two peptides formed are held together by one or more S-S bridges.

The precise cleavage site(s) in respect of the post-translational and/or post-transcriptional modification may vary slightly such that by way of example only the 38 amino acids removed (as shown in SEQ ID No. 68 compared with SEQ ID No. 16) may vary slightly. Without wishing to be bound by theory, the cleavage site may be shifted by a few residues (e.g. 1, 2 or 3 residues) in either direction compared with the cleavage site shown by reference to SEQ ID No. 68 compared with SEQ ID No. 16. In other words, rather than cleavage at position 235-ATR to position 273 (RRSAS) for example, the cleavage may commence at residue 232, 233, 234, 235, 236, 237 or 238 for example. In addition or alternatively, the cleavage may result in the removal of about 38 amino acids, in some embodiments the cleavage may result in the removal of between 30-45 residues, such as 34-42 residues, such as 36-40 residues, preferably 38 residues.

### Isolated

In one aspect, the lipid acyltransferase is a recovered/isolated lipid acyltransferase. Thus, the lipid acyltransferase produced may be in an isolated form.

In another aspect, the nucleotide sequence encoding a lipid acyltransferase for use in the present invention may be in an isolated form.

The term “isolated” means that the sequence or protein is at least substantially free from at least one other component with which the sequence or protein is naturally associated in nature and as found in nature.

In one aspect the phytosterol ester and/or phytostanol ester may be isolated or separated from the other constituents of the reaction admixture or reaction composition. In this regard, the term “isolated” or “isolating” means that the phytosterol ester and/or phytostanol ester is at least substantially free from at least one other component) found in the reaction admixture or reaction composition or is treated to render it at least substantially free from at least one other component found in the reaction admixture or reaction composition.

In one aspect the phytosterol ester and/or phytostanol ester is in an isolated form.

### Purified

In one aspect, the lipid acyltransferase may be in a purified form.

In another aspect, the nucleotide sequence encoding a lipid acyltransferase for use in the present invention may be in a purified form.

In a further aspect the phytosterol ester and/or phytostanol ester may be in a purified form.

The term “purified” means that the enzyme or the phytostanol ester or phytosterol ester is in a relatively pure state—e.g. at least about 51% pure, or at least about 75%, or at least about 80%, or at least about 90% pure, or at least about 95% pure or at least about 98% pure.

In one aspect the term “purifying” means that the phytostanol ester and/or phytosterol ester is treated to render it in a relatively pure state—e.g. at least about 51% pure, or at least about 75%, or at least about 80%, or at least about 90% pure, or at least about 95% pure or at least about 98% pure.

### Foodstuff

The term “foodstuff” as used herein means a substance which is suitable for human and/or animal consumption. Hence the term “food” or “foodstuff” used herein includes “feed” and a “feedstuff”.

Suitably, the term “foodstuff” as used herein may mean a foodstuff in a form which is ready for consumption. Alternatively or in addition, however, the term foodstuff as used herein may mean one or more food materials which are used in the preparation of a foodstuff. By way of example only, the term foodstuff encompasses both baked goods produced from dough as well as the dough used in the preparation of said baked goods.

In a preferred aspect the present invention provides a foodstuff as defined above wherein the foodstuff is selected from one or more of the following: eggs, egg-based products, including but not limited to mayonnaise, salad dressings, sauces, ice creams, egg powder, modified egg yolk and products made therefrom; baked goods, including breads, cakes, sweet dough products, laminated doughs, liquid batters, muffins, doughnuts, biscuits, crackers and cookies; confectionery, including chocolate, candies, caramels, halawa, gums, including sugar free and sugar sweetened gums, bubble gum, soft bubble gum, chewing gum and puddings; frozen products including sorbets, preferably frozen dairy products, including ice cream and ice milk; dairy products, including cheese, butter, milk, coffee cream, whipped cream, custard cream, milk drinks and yoghurts; mousses, whipped vegetable creams, meat products, including processed meat products; edible oils and fats, aerated and non-aerated whipped products, oil-in-water emulsions, water-in-oil emulsions, margarine, shortening and spreads including low fat and very low fat spreads; dressings, mayonnaise, dips, cream based sauces, cream based soups, beverages, spice emulsions and sauces.

Suitably the foodstuff in accordance with the present invention may be a “fine foods”, including cakes, pastry, confectionery, chocolates, fudge and the like.

In one aspect the foodstuff in accordance with the present invention may be a, dough product or a baked product, such as a bread, a fried product, a snack, cakes, pies, brownies, cookies, noodles, snack items such as crackers, graham crackers, pretzels, and potato chips, and pasta.

In a further aspect, the foodstuff in accordance with the present invention may be a plant derived food product such as flours, pre-mixes, oils, fats, cocoa butter, coffee whitener, salad dressings, margarine, spreads, peanut butter, shortenings, ice cream, cooking oils.

In another aspect, the foodstuff in accordance with the present invention may be a dairy product, including butter, milk, cream, cheese such as natural, processed, and imitation cheeses in a variety of forms (including shredded, block, slices or grated), cream cheese, ice cream, frozen desserts, yoghurt, yoghurt drinks, butter fat, anhydrous milk fat, other dairy products.

In another aspect, the foodstuff in accordance with the present invention may be a food product containing animal derived ingredients, such as processed meat products, cooking oils, shortenings.

In a further aspect, the foodstuff in accordance with the present invention may be a beverage, a fruit, mixed fruit, a vegetable or wine. In some cases the beverage may contain up to 20 g/l of added phytosterol esters.

In another aspect, the foodstuff in accordance with the present invention may be an animal feed. The animal feed may be enriched with phytosterol esters and/or phytostanol esters, preferably with beta-sitosterol/stanol ester. Suitably, the animal feed may be a poultry feed. When the foodstuff is poultry feed, the present invention may be used to lower the cholesterol content of eggs produced by poultry fed on the foodstuff according to the present invention.

In one aspect the foodstuff may be selected from one or more of the following: eggs, egg-based products, including mayonnaise, salad dressings, sauces, ice cream, egg powder, modified egg yolk and products made therefrom.

In a further aspect foodstuff is preferably a margarine or mayonnaise.

The term “food material” as used herein means at least one component or at least one ingredient of a foodstuff.

### Personal Care Products

Phytosterols and phytostanols are compounds with strong dermatological (anti-inflammatory and anti-erythemal) and biological (hyptcholesterolemic) activity and are of interest for dermo-cosmetics and nutrition products.

The phytosterol esters and/or phytostanol esters prepared by the method and uses of the present invention include any cosmetic product or cosmetic emulsion for human use, including soaps, skin creams, facial creams, face masks, skin cleanser, tooth paste, lipstick, perfumes, make-up, foundation, blusher, mascara, eyeshadow, sunscreen lotions, hair conditioner, and hair colouring.

### Pharmaceutical Compositions

The present invention also provides a pharmaceutical composition comprising a sterol esters and/or stanol esters produced by methods or uses of the present invention and a pharmaceutically acceptable carrier, diluent or excipient (including combinations thereof).

The pharmaceutical compositions may be for human or animal usage in human and veterinary medicine and will typically comprise any one or more of a pharmaceutically acceptable diluent, carrier, or excipient. Acceptable carriers or diluents for therapeutic use are well known in the pharmaceutical art, and are described, for example, in Remington's Pharmaceutical Sciences, Mack Publishing Co. (A. R. Gennaro edit. 1985). The choice of pharmaceutical carrier, excipient or diluent can be selected with regard to the intended route of administration and standard pharmaceutical practice. The pharmaceutical compositions may comprise as—or in addition to—the carrier, excipient or diluent any suitable binder(s), lubricant(s), suspending agent(s), coating agent(s), solubilising agent(s).

Preservatives, stabilisers, dyes and even flavouring agents may be provided in the pharmaceutical composition. Examples of preservatives include sodium benzoate, sorbic acid and esters of p-hydroxybenzoic acid. Antioxidants and suspending agents may be also used.

There may be different composition/formulation requirements dependent on the different delivery systems. By way of example, the pharmaceutical composition of the present invention may be formulated to be delivered using a mini-pump or by a mucosal route, for example, as a nasal spray or aerosol for inhalation or ingestable solution, or parenterally in which the composition is formulated by an injectable form, for delivery, by, for example, an intravenous, intramuscular or subcutaneous route. Alternatively, the formulation may be designed to be delivered by both routes.

Where the agent is to be delivered mucosally through the gastrointestinal mucosa, it should be able to remain stable during transit though the gastrointestinal tract; for example, it should be resistant to proteolytic degradation, stable at acid pH and resistant to the detergent effects of bile.

Where appropriate, the pharmaceutical compositions can be administered by inhalation, in the form of a suppository or pessary, topically in the form of a lotion, solution, cream, ointment or dusting powder, by use of a skin patch, orally in the form of tablets containing excipients such as starch or lactose, or in capsules or ovules either alone or in admixture with excipients, or in the form of elixirs, solutions or suspensions containing flavouring or colouring agents, or they can be injected parenterally, for example intravenously, intramuscularly or subcutaneously. For parenteral administration, the compositions may be best used in the form of a sterile aqueous solution which may contain other substances, for example enough salts or monosaccharides to make the solution isotonic with blood. For buccal or sublingual administration the compositions may be administered in the form of tablets or lozenges which can be formulated in a conventional manner.

Preferably the pharmaceutical composition is in a form that is suitable for oral delivery.

### Cloning a Nucleotide Sequence Encoding a Polypeptide According to the Present Invention

A nucleotide sequence encoding either a polypeptide which has the specific properties as defined herein or a polypeptide which is suitable for modification may be isolated from any cell or organism producing said polypeptide. Various methods are well known within the art for the isolation of nucleotide sequences.

For example, a genomic DNA and/or cDNA library may be constructed using chromosomal DNA or messenger RNA from the organism producing the polypeptide. If the amino acid sequence of the polypeptide is known, labeled oligonucleotide probes may be synthesised and used to identify polypeptide-encoding clones from the genomic library prepared from the organism. Alternatively, a labelled oligonucleotide probe containing sequences homologous to another known polypeptide gene could be used to identify polypeptide-encoding clones. In the latter case, hybridisation and washing conditions of lower stringency are used.

Alternatively, polypeptide-encoding clones could be identified by inserting fragments of genomic DNA into an expression vector, such as a plasmid, transforming enzyme-negative bacteria with the resulting genomic DNA library, and then plating the transformed bacteria onto agar containing an enzyme inhibited by the polypeptide, thereby allowing clones expressing the polypeptide to be identified.

In a yet further alternative, the nucleotide sequence encoding the polypeptide may be prepared synthetically by established standard methods, e.g. the phosphoroamidite method described by Beucage S. L. et al (1981) Tetrahedron Letters 22, p 1859-1869, or the method described by Matthes et al (1984) EMBO J. 3, p 801-805. In the phosphoroamidite method, oligonucleotides are synthesised, e.g. in an automatic DNA synthesiser, purified, annealed, ligated and cloned in appropriate vectors.

The nucleotide sequence may be of mixed genomic and synthetic origin, mixed synthetic and cDNA origin, or mixed genomic and cDNA origin, prepared by ligating fragments of synthetic, genomic or cDNA origin (as appropriate) in accordance with standard techniques. Each ligated fragment corresponds to various parts of the entire nucleotide sequence. The DNA sequence may also be prepared by polymerase chain reaction (PCR) using specific primers, for instance as described in U.S. Pat. No. 4,683,202 or in Saiki R K et al (Science (1988) 239, pp 487-491).

### Nucleotide Sequences

The present invention also encompasses nucleotide sequences encoding polypeptides having the specific properties as defined herein. The term “nucleotide sequence” as used herein refers to an oligonucleotide sequence or polynucleotide sequence, and variant, homologues, fragments and derivatives thereof (such as portions thereof). The nucleotide sequence may be of genomic or synthetic or recombinant origin, which may be double-stranded or single-stranded whether representing the sense or antisense strand.

The term “nucleotide sequence” in relation to the present invention includes genomic DNA, cDNA, synthetic DNA, and RNA. Preferably it means DNA, more preferably cDNA for the coding sequence.

In a preferred embodiment, the nucleotide sequence per se encoding a polypeptide having the specific properties as defined herein does not cover the native nucleotide sequence in its natural environment when it is linked to its naturally associated sequence(s) that is/are also in its/their natural environment. For ease of reference, we shall call this preferred embodiment the “non-native nucleotide sequence”. In this regard, the term “native nucleotide sequence” means an entire nucleotide sequence that is in its native environment and when operatively linked to an entire promoter with which it is naturally associated, which promoter is also in its native environment. Thus, the polypeptide of the present invention can be expressed by a nucleotide sequence in its native organism but wherein the nucleotide sequence is not under the control of the promoter with which it is naturally associated within that organism.

Preferably the polypeptide is not a native polypeptide. In this regard, the term “native polypeptide” means an entire polypeptide that is in its native environment and when it has been expressed by its native nucleotide sequence.

Typically, the nucleotide sequence encoding polypeptides having the specific properties as defined herein is prepared using recombinant DNA techniques (i.e. recombinant DNA). However, in an alternative embodiment of the invention, the nucleotide sequence could be synthesised, in whole or in part, using chemical methods well known in the art (see Caruthers M H et al (1980) Nuc Acids Res Symp Ser 215-23 and Horn T et al (1980) Nuc Acids Res Symp Ser 225-232).

### Molecular Evolution

Once an enzyme-encoding nucleotide sequence has been isolated, or a putative enzyme-encoding nucleotide sequence has been identified, it may be desirable to modify the selected nucleotide sequence, for example it may be desirable to mutate the sequence in order to prepare an enzyme in accordance with the present invention.

Mutations may be introduced using synthetic oligonucleotides. These oligonucleotides contain nucleotide sequences flanking the desired mutation sites.

A suitable method is disclosed in Morinaga et al (Biotechnology (1984) 2, p 646-649). Another method of introducing mutations into enzyme-encoding nucleotide sequences is described in Nelson and Long (Analytical Biochemistry (1989), 180, p 147-151).

Instead of site directed mutagenesis, such as described above, one can introduce mutations randomly for instance using a commercial kit such as the GeneMorph PCR mutagenesis kit from Stratagene, or the Diversify PCR random mutagenesis kit from Clontech. EP 0 583 265 refers to methods of optimising PCR based mutagenesis, which can also be combined with the use of mutagenic DNA analogues such as those described in EP 0 866 796. Error prone PCR technologies are suitable for the production of variants of lipid acyl transferases with preferred characteristics. WO0206457 refers to molecular evolution of lipases.

A third method to obtain novel sequences is to fragment non-identical nucleotide sequences, either by using any number of restriction enzymes or an enzyme such as Dnase I, and reassembling full nucleotide sequences coding for functional proteins. Alternatively one can use one or multiple non-identical nucleotide sequences and introduce mutations during the reassembly of the full nucleotide sequence. DNA shuffling and family shuffling technologies are suitable for the production of variants of lipid acyl transferases with preferred characteristics. Suitable methods for performing ‘shuffling’ can be found in EP0 752 008, EP1 138 763, EP1 103 606. Shuffling can also be combined with other forms of DNA mutagenesis as described in U.S. Pat. No. 6,180,406 and WO 01/34835.

Thus, it is possible to produce numerous site directed or random mutations into a nucleotide sequence, either in vivo or in vitro, and to subsequently screen for improved functionality of the encoded polypeptide by various means. Using in silico and exo mediated recombination methods (see WO 00/58517, U.S. Pat. No. 6,344,328, U.S. Pat. No. 6,361,974), for example, molecular evolution can be performed where the variant produced retains very low homology to known enzymes or proteins. Such variants thereby obtained may have significant structural analogy to known transferase enzymes, but have very low amino acid sequence homology.

As a non-limiting example, in addition, mutations or natural variants of a polynucleotide sequence can be recombined with either the wild type or other mutations or natural variants to produce new variants. Such new variants can also be screened for improved functionality of the encoded polypeptide.

The application of the above-mentioned and similar molecular evolution methods allows the identification and selection of variants of the enzymes of the present invention which have preferred characteristics without any prior knowledge of protein structure or function, and allows the production of non-predictable but beneficial mutations or variants. There are numerous examples of the application of molecular evolution in the art for the optimisation or alteration of enzyme activity, such examples include, but are not limited to one or more of the following: optimised expression and/or activity in a host cell or in vitro, increased enzymatic activity, altered substrate and/or product specificity, increased or decreased enzymatic or structural stability, altered enzymatic activity/specificity in preferred environmental conditions, e.g. temperature, pH, substrate

As will be apparent to a person skilled in the art, using molecular evolution tools an enzyme may be altered to improve the functionality of the enzyme.

Suitably, the nucleotide sequence encoding a lipid acyltransferase used in the invention may encode a variant lipid acyltransferase, i.e. the lipid acyltransferase may contain at least one amino acid substitution, deletion or addition, when compared to a parental enzyme. Variant enzymes retain at least 1%, 2%, 3%, 5%, 10%, 15%, 20%, 30%, 40%, 50%, 60%, 70%, 80%, 90%, 95%, 97%, 99% identity with the parent enzyme. Suitable parent enzymes may include any enzyme with esterase or lipase activity. Preferably, the parent enzyme aligns to the pfam00657 consensus sequence.

In a preferable embodiment a variant lipid acyltransferase enzyme retains or incorporates at least one or more of the pfam00657 consensus sequence amino acid residues found in the GDSX, GANDY and HPT blocks.

Enzymes, such as lipases with no or low lipid acyltransferase activity in an aqueous environment may be mutated using molecular evolution tools to introduce or enhance the transferase activity, thereby producing a lipid acyltransferase enzyme with significant transferase activity suitable for use in the compositions and methods of the present invention.

Suitably, the nucleotide sequence encoding a lipid acyltransferase for use in any one of the methods and/or uses of the present invention may encode a lipid acyltransferase that may be a variant with enhanced enzyme activity on polar lipids, preferably phospholipids when compared to the parent enzyme.

Alternatively, the variant enzyme may have increased thermostability.

Variants of lipid acyltransferases are known, and one or more of such variants may be suitable for use in the methods and uses according to the present invention and/or in the enzyme compositions according to the present invention. By way of example only, variants of lipid acyltransferases are described in the following references may be used in accordance with the present invention: Hilton & Buckley J. Biol. Chem. 1991 Jan. 15: 266 (2): 997-1000; Robertson et al J. Biol. Chem. 1994 Jan. 21; 269(3):2146-50; Brumlik et al J. Bacteriol 1996 April; 178 (7): 2060-4; Peelman et al Protein Sci. 1998 March; 7(3):587-99.

### Amino Acid Sequences

The present invention also encompasses the use of amino acid sequences encoded by a nucleotide sequence which encodes a lipid acyltransferase for use in any one of the methods and/or uses of the present invention.

As used herein, the term “amino acid sequence” is synonymous with the term “polypeptide” and/or the term “protein”. In some instances, the term “amino acid sequence” is synonymous with the term “peptide”.

The amino acid sequence may be prepared/isolated from a suitable source, or it may be made synthetically or it may be prepared by use of recombinant DNA techniques.

Suitably, the amino acid sequences may be obtained from the isolated polypeptides taught herein by standard techniques.

One suitable method for determining amino acid sequences from isolated polypeptides is as follows:
Purified polypeptide may be freeze-dried and 100 μg of the freeze-dried material may be dissolved in 50 μl of a mixture of 8 M urea and 0.4 M ammonium hydrogen carbonate, pH 8.4. The dissolved protein may be denatured and reduced for 15 minutes at 50° C. following overlay with nitrogen and addition of 5 μl of 45 mM dithiothreitol. After cooling to room temperature, 5 μl of 100 mM iodoacetamide may be added for the cysteine residues to be derivatized for 15 minutes at room temperature in the dark under nitrogen.
135 μl of water and 5 μg of endoproteinase Lys-C in 5 μl of water may be added to the above reaction mixture and the digestion may be carried out at 37° C. under nitrogen for 24 hours.

The resulting peptides may be separated by reverse phase HPLC on a VYDAC C18 column (0.46×15 cm; 10 μm; The Separation Group, California, USA) using solvent A: 0.1% TFA in water and solvent B: 0.1% TFA in acetonitrile. Selected peptides may be re-chromatographed on a Develosil C18 column using the same solvent system, prior to N-terminal sequencing. Sequencing may be done using an Applied Biosystems 476A sequencer using pulsed liquid fast cycles according to the manufacturer's instructions (Applied Biosystems, California, USA).

### Sequence Identity or Sequence Homology

Here, the term “homologue” means an entity having a certain homology with the subject amino acid sequences and the subject nucleotide sequences. Here, the term “homology” can be equated with “identity”.

The homologous amino acid sequence and/or nucleotide sequence should provide and/or encode a polypeptide which retains the functional activity and/or enhances the activity of the enzyme.

In the present context, a homologous sequence is taken to include an amino acid sequence which may be at least 75, 85 or 90% identical, preferably at least 95 or 98% identical to the subject sequence. Typically, the homologues will comprise the same active sites etc. as the subject amino acid sequence. Although homology can also be considered in terms of similarity (i.e. amino acid residues having similar chemical properties/functions), in the context of the present invention it is preferred to express homology in terms of sequence identity.

In the present context, a homologous sequence is taken to include a nucleotide sequence which may be at least 75, 85 or 90% identical, preferably at least 95 or 98% identical to a nucleotide sequence encoding a polypeptide of the present invention (the subject sequence). Typically, the homologues will comprise the same sequences that code for the active sites etc. as the subject sequence. Although homology can also be considered in terms of similarity (i.e. amino acid residues having similar chemical properties/functions), in the context of the present invention it is preferred to express homology in terms of sequence identity.

Homology comparisons can be conducted by eye, or more usually, with the aid of readily available sequence comparison programs. These commercially available computer programs can calculate % homology between two or more sequences.

% homology may be calculated over contiguous sequences, i.e. one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion will cause the following amino acid residues to be put out of alignment, thus potentially resulting in a large reduction in % homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible—reflecting higher relatedness between the two compared sequences—will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons.

Calculation of maximum % homology therefore firstly requires the production of an optimal alignment, taking into consideration gap penalties. A suitable computer program for carrying out such an alignment is the Vector NTI Advance™ 11 (Invitrogen Corp.). Examples of other software that can perform sequence comparisons include, but are not limited to, the BLAST package (see Ausubel et al 1999 Short Protocols in Molecular Biology, 4th Ed—Chapter 18), and FASTA (Altschul et al 1990 J. Mol. Biol. 403-410). Both BLAST and FASTA are available for offline and online searching (see Ausubel et al 1999, pages 7-58 to 7-60). However, for some applications, it is preferred to use the Vector NTI Advance™ 11 program. A new tool, called BLAST 2 Sequences is also available for comparing protein and nucleotide sequence (see FEMS Microbiol Lett 1999 174(2): 247-50; and FEMS Microbiol Lett 1999 177(1): 187-8).

Although the final % homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. Vector NTI programs generally use either the public default values or a custom symbol comparison table if supplied (see user manual for further details). For some applications, it is preferred to use the default values for the Vector NTI Advance™ 11 package.

Alternatively, percentage homologies may be calculated using the multiple alignment feature in Vector NTI Advance™ 11 (Invitrogen Corp.), based on an algorithm, analogous to CLUSTAL (Higgins D G & Sharp P M (1988), Gene 73(1), 237-244).

Once the software has produced an optimal alignment, it is possible to calculate % homology, preferably % sequence identity. The software typically does this as part of the sequence comparison and generates a numerical result.

Should Gap Penalties be used when determining sequence identity, then preferably the default parameters for the programme are used for pairwise alignment. For example, the following parameters are the current default parameters for pairwise alignment for BLAST 2:

| FOR BLAST2 | DNA | PROTEIN |
|---|---|---|
| EXPECT THRESHOLD | 10 | 10 |
| WORD SIZE | 11 | 3 |
| SCORING PARAMETERS | | |
| Match/Mismatch Scores | 2, −3 | n/a |
| Matrix | n/a | BLOSUM62 |
| Gap Costs | Existence: 5 | Existence: 11 |
| | Extension: 2 | Extension: 1 |

In one embodiment, preferably the sequence identity for the nucleotide sequences and/or amino acid sequences may be determined using BLAST2 (blastn) with the scoring parameters set as defined above.

For the purposes of the present invention, the degree of identity is based on the number of sequence elements which are the same. The degree of identity in accordance with the present invention for amino acid sequences may be suitably determined by means of computer programs known in the art such as Vector NTI Advance™ 11 (Invitrogen Corp.). For pairwise alignment the scoring parameters used are preferably BLOSUM62 with Gap existence penalty of 11 and Gap extension penalty of 1.

Suitably, the degree of identity with regard to a nucleotide sequence is determined over at least 20 contiguous nucleotides, preferably over at least 30 contiguous nucleotides, preferably over at least 40 contiguous nucleotides, preferably over at least 50 contiguous nucleotides, preferably over at least 60 contiguous nucleotides, preferably over at least 100 contiguous nucleotides.

Suitably, the degree of identity with regard to a nucleotide sequence may be determined over the whole sequence.

The sequences may also have deletions, insertions or substitutions of amino acid residues which produce a silent change and result in a functionally equivalent substance. Deliberate amino acid substitutions may be made on the basis of similarity in polarity, charge, solubility, hydrophobicity, hydrophilicity, and/or the amphipathic nature of the residues as long as the secondary binding activity of the substance is retained. For example, negatively charged amino acids include aspartic acid and glutamic acid; positively charged amino acids include lysine and arginine; and amino acids with uncharged polar head groups having similar hydrophilicity values include leucine, isoleucine, valine, glycine, alanine, asparagine, glutamine, serine, threonine, phenylalanine, and tyrosine.

Conservative substitutions may be made, for example according to the Table below. Amino acids in the same block in the second column and preferably in the same line in the third column may be substituted for each other:

| | | |
|---|---|---|
| ALIPHATIC | Non-polar | G A P |
| | | I L V |
| | Polar—uncharged | C S T M |
| | | N Q |
| | Polar—charged | D E |
| | | K R |
| AROMATIC | | H F W Y |

The present invention also encompasses homologous substitution (substitution and replacement are both used herein to mean the interchange of an existing amino acid residue, with an alternative residue) that may occur i.e. like-for-like substitution such as basic for basic, acidic for acidic, polar for polar etc. Non-homologous substitution may also occur i.e. from one class of residue to another or alternatively involving the inclusion of unnatural amino acids such as ornithine (hereinafter referred to as Z), diaminobutyric acid ornithine (hereinafter referred to as B), norleucine ornithine (hereinafter referred to as O), pyriylalanine, thienylalanine, naphthylalanine and phenylglycine.

Replacements may also be made by unnatural amino acids.

Variant amino acid sequences may include suitable spacer groups that may be inserted between any two amino acid residues of the sequence including alkyl groups such as methyl, ethyl or propyl groups in addition to amino acid spacers such as glycine or β-alanine residues. A further form of variation, involves the presence of one or more amino acid residues in peptoid form, will be well understood by those skilled in the art. For the avoidance of doubt, “the peptoid form” is used to refer to variant amino acid residues wherein the α-carbon substituent group is on the residue's nitrogen atom rather than the α-carbon. Processes for preparing peptides in the peptoid form are known in the art, for example Simon R J et al., PNAS (1992) 89(20), 9367-9371 and Horwell D C, Trends Biotechnol. (1995) 13(4), 132-134.

Nucleotide sequences for use in the present invention or encoding a polypeptide having the specific properties defined herein may include within them synthetic or modified nucleotides. A number of different types of modification to oligonucleotides are known in the art. These include methylphosphonate and phosphorothioate backbones and/or the addition of acridine or polylysine chains at the 3′ and/or 5′ ends of the molecule. For the purposes of the present invention, it is to be understood that the nucleotide sequences described herein may be modified by any method available in the art. Such modifications may be carried out in order to enhance the in vivo activity or life span of nucleotide sequences.

The present invention also encompasses the use of nucleotide sequences that are complementary to the sequences discussed herein, or any derivative, fragment or derivative thereof. If the sequence is complementary to a fragment thereof then that sequence can be used as a probe to identify similar coding sequences in other organisms etc.

Polynucleotides which are not 100% homologous to the sequences of the present invention but fall within the scope of the invention can be obtained in a number of ways. Other variants of the sequences described herein may be obtained for example by probing DNA libraries made from a range of individuals, for example individuals from different populations. In addition, other viral/bacterial, or cellular homologues particularly cellular homologues found in mammalian cells (e.g. rat, mouse, bovine and primate cells), may be obtained and such homologues and fragments thereof in general will be capable of selectively hybridising to the sequences shown in the sequence listing herein. Such sequences may be obtained by probing cDNA libraries made from or genomic DNA libraries from other animal species, and probing such libraries with probes comprising all or part of any one of the sequences in the attached sequence listings under conditions of medium to high stringency. Similar considerations apply to obtaining species homologues and allelic variants of the polypeptide or nucleotide sequences of the invention.

Variants and strain/species homologues may also be obtained using degenerate PCR which will use primers designed to target sequences within the variants and homologues encoding conserved amino acid sequences within the sequences of the present invention. Conserved sequences can be predicted, for example, by aligning the amino acid sequences from several variants/homologues. Sequence alignments can be performed using computer software known in the art. For example the GCG Wisconsin PileUp program is widely used.

The primers used in degenerate PCR will contain one or more degenerate positions and will be used at stringency conditions lower than those used for cloning sequences with single sequence primers against known sequences.

Alternatively, such polynucleotides may be obtained by site directed mutagenesis of characterised sequences. This may be useful where for example silent codon sequence changes are required to optimise codon preferences for a particular host cell in which the polynucleotide sequences are being expressed. Other sequence changes may be desired in order to introduce restriction polypeptide recognition sites, or to alter the property or function of the polypeptides encoded by the polynucleotides.

Polynucleotides (nucleotide sequences) of the invention may be used to produce a primer, e.g. a PCR primer, a primer for an alternative amplification reaction, a probe e.g. labelled with a revealing label by conventional means using radioactive or non-radioactive labels, or the polynucleotides may be cloned into vectors. Such primers, probes and other fragments will be at least 15, preferably at least 20, for example at least 25, 30 or 40 nucleotides in length, and are also encompassed by the term polynucleotides of the invention as used herein.

Polynucleotides such as DNA polynucleotides and probes according to the invention may be produced recombinantly, synthetically, or by any means available to those of skill in the art. They may also be cloned by standard techniques.

In general, primers will be produced by synthetic means, involving a stepwise manufacture of the desired nucleic acid sequence one nucleotide at a time. Techniques for accomplishing this using automated techniques are readily available in the art.

Longer polynucleotides will generally be produced using recombinant means, for example using a PCR (polymerase chain reaction) cloning techniques. This will involve making a pair of primers (e.g. of about 15 to 30 nucleotides) flanking a region of the lipid targeting sequence which it is desired to clone, bringing the primers into contact with mRNA or cDNA obtained from an animal or human cell, performing a polymerase chain reaction under conditions which bring about amplification of the desired region, isolating the amplified fragment (e.g. by purifying the reaction mixture on an agarose gel) and recovering the amplified DNA. The primers may be designed to contain suitable restriction enzyme recognition sites so that the amplified DNA can be cloned into a suitable cloning vector.

### Hybridisation

The present invention also encompasses the use of sequences that are complementary to the sequences of the present invention or sequences that are capable of hybridising either to the sequences of the present invention or to sequences that are complementary thereto.

The term “hybridisation” as used herein shall include “the process by which a strand of nucleic acid joins with a complementary strand through base pairing” as well as the process of amplification as carried out in polymerase chain reaction (PCR) technologies.

The present invention also encompasses the use of nucleotide sequences that are capable of hybridising to the sequences that are complementary to the subject sequences discussed herein, or any derivative, fragment or derivative thereof.

The present invention also encompasses sequences that are complementary to sequences that are capable of hybridising to the nucleotide sequences discussed herein.

Hybridisation conditions are based on the melting temperature (Tm) of the nucleotide binding complex, as taught in Berger and Kimmel (1987, Guide to Molecular Cloning Techniques, Methods in Enzymology, Vol. 152, Academic Press, San Diego Calif.), and confer a defined “stringency” as explained below.

Maximum stringency typically occurs at about Tm-5° C. (5° C. below the Tm of the probe); high stringency at about 5° C. to 10° C. below Tm; intermediate stringency at about 10° C. to 20° C. below Tm; and low stringency at about 20° C. to 25° C. below Tm. As will be understood by those of skill in the art, a maximum stringency hybridisation can be used to identify or detect identical nucleotide sequences while an intermediate (or low) stringency hybridisation can be used to identify or detect similar or related polynucleotide sequences.

Preferably, the present invention encompasses the use of sequences that are complementary to sequences that are capable of hybridising under high stringency conditions or intermediate stringency conditions to nucleotide sequences encoding polypeptides having the specific properties as defined herein.

More preferably, the present invention encompasses the use of sequences that are complementary to sequences that are capable of hybridising under high stringency conditions (e.g. 65° C. and 0.1×SSC {1×SSC=0.15 M NaCl, 0.015 M Na-citrate pH 7.0}) to nucleotide sequences encoding polypeptides having the specific properties as defined herein.

The present invention also relates to the use of nucleotide sequences that can hybridise to the nucleotide sequences discussed herein (including complementary sequences of those discussed herein).

The present invention also relates to the use of nucleotide sequences that are complementary to sequences that can hybridise to the nucleotide sequences discussed herein (including complementary sequences of those discussed herein).

Also included within the scope of the present invention are the use of polynucleotide sequences that are capable of hybridising to the nucleotide sequences discussed herein under conditions of intermediate to maximal stringency.

In a preferred aspect, the present invention covers the use of nucleotide sequences that can hybridise to the nucleotide sequences discussed herein, or the complement thereof, under stringent conditions (e.g. 50° C. and 0.2×SSC).

In a more preferred aspect, the present invention covers the use of nucleotide sequences that can hybridise to the nucleotide sequences discussed herein, or the complement thereof, under high stringency conditions (e.g. 65° C. and 0.1×SSC).

### Expression of Polypeptides

A nucleotide sequence for use in the present invention or for encoding a polypeptide having the specific properties as defined herein can be incorporated into a recombinant replicable vector. The vector may be used to replicate and express the nucleotide sequence, in polypeptide form, in and/or from a compatible host cell. Expression may be controlled using control sequences which include promoters/enhancers and other expression regulation signals. Prokaryotic promoters and promoters functional in eukaryotic cells may be used. Tissue specific or stimuli specific promoters may be used. Chimeric promoters may also be used comprising sequence elements from two or more different promoters described above.

The polypeptide produced by a host recombinant cell by expression of the nucleotide sequence may be secreted or may be contained intracellularly depending on the sequence and/or the vector used. The coding sequences can be designed with signal sequences which direct secretion of the substance coding sequences through a particular prokaryotic or eukaryotic cell membrane.

### Constructs

The term “construct”—which is synonymous with terms such as “conjugate”, “cassette” and “hybrid”—includes a nucleotide sequence encoding a polypeptide having the specific properties as defined herein for use according to the present invention directly or indirectly attached to a promoter. An example of an indirect attachment is the provision of a suitable spacer group such as an intron sequence, such as the Sh1-intron or the ADH intron, intermediate the promoter and the nucleotide sequence of the present invention. The same is true for the term “fused” in relation to the present invention which includes direct or indirect attachment. In some cases, the terms do not cover the natural combination of the nucleotide sequence coding for the protein ordinarily associated with the wild type gene promoter and when they are both in their natural environment.

The construct may even contain or express a marker which allows for the selection of the genetic construct.

For some applications, preferably the construct comprises at least a nucleotide sequence of the present invention or a nucleotide sequence encoding a polypeptide having the specific properties as defined herein operably linked to a promoter.

### Organism

The term “organism” in relation to the present invention includes any organism that could comprise a nucleotide sequence according to the present invention or a nucleotide sequence encoding for a polypeptide having the specific properties as defined herein and/or products obtained therefrom.

The term “transgenic organism” in relation to the present invention includes any organism that comprises a nucleotide sequence coding for a polypeptide having the specific properties as defined herein and/or the products obtained therefrom, and/or wherein a promoter can allow expression of the nucleotide sequence coding for a polypeptide having the specific properties as defined herein within the organism. Preferably the nucleotide sequence is incorporated in the genome of the organism.

The term “transgenic organism” does not cover native nucleotide coding sequences in their natural environment when they are under the control of their native promoter which is also in its natural environment.

Therefore, the transgenic organism of the present invention includes an organism comprising any one of, or combinations of, a nucleotide sequence coding for a polypeptide having the specific properties as defined herein, constructs as defined herein, vectors as defined herein, plasmids as defined herein, cells as defined herein, or the products thereof. For example the transgenic organism can also comprise a nucleotide sequence coding for a polypeptide having the specific properties as defined herein under the control of a promoter not associated with a sequence encoding a lipid acyltransferase in nature.

### Host Cell

The lipid acyltransferase may be produced by expression of a nucleotide sequence in a host organism wherein the host organism can be a prokaryotic or a eukaryotic organism.

In one embodiment of the present invention the lipid acyl transferase according to the present invention in expressed in a host cell, for example a bacterial cells, such as a Bacillus spp, for example a Bacillus licheniformis host cell (as taught in WO2008/090395—incorporated herein by reference).

Alternative host cells may be fungi, yeasts or plants for example.

### Transformation of Host Cells/Organism

The host organism can be a prokaryotic or a eukaryotic organism.

Examples of suitable prokaryotic hosts include bacteria such as E. coli and Bacillus licheniformis, preferably B. licheniformis. Transformation of B. licheniformis with nucleotide sequences encoding lipid acyltransferases is taught in WO2008/090395—incorporated herein by reference.

Teachings on the transformation of other prokaryotic hosts is well documented in the art, for example see Sambrook et al (Molecular Cloning: A Laboratory Manual, 2nd edition, 1989, Cold Spring Harbor Laboratory Press). If a prokaryotic host is used then the nucleotide sequence may need to be suitably modified before transformation—such as by removal of introns.

In another embodiment the transgenic organism can be a yeast.

Filamentous fungi cells may be transformed using various methods known in the art—such as a process involving protoplast formation and transformation of the protoplasts followed by regeneration of the cell wall in a manner known. The use of Aspergillus as a host microorganism is described in EP 0 238 023.

Another host organism can be a plant. A review of the general techniques used for transforming plants may be found in articles by Potrykus (Annu Rev Plant Physiol Plant Mol Biol [1991] 42:205-225) and Christou (Agro-Food-Industry Hi-Tech March/April 1994 17-27). Further teachings on plant transformation may be found in EP-A-0449375.

The invention will now be described, by way of example only, with reference to the following Figures and Examples.

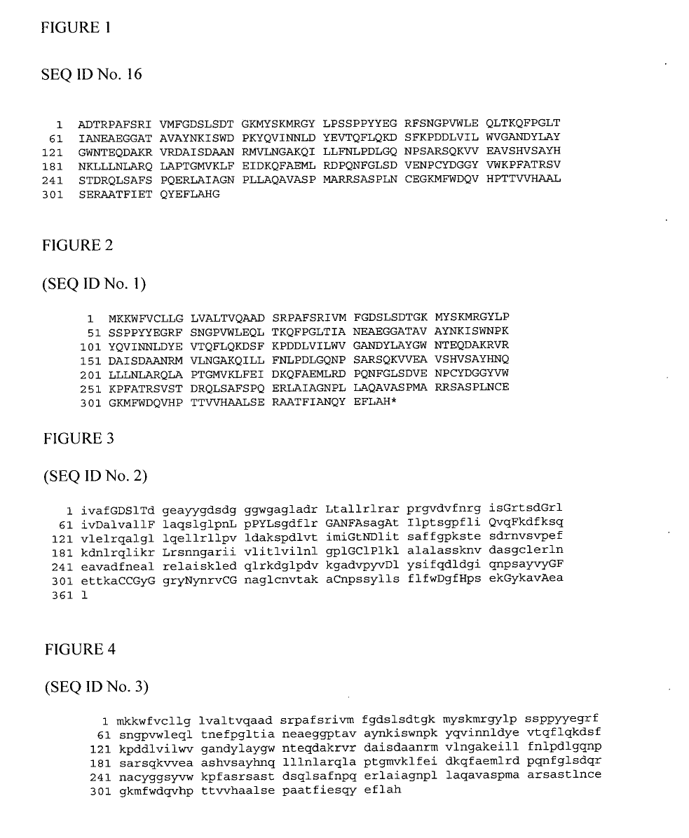

FIG. 1 shows the amino acid sequence of a mutant Aeromonas salmonicida mature lipid acyltransferase (GCAT) with a mutation of Asn80Asp (notably, amino acid 80 is in the mature sequence) (SEQ ID 16);

FIG. 2 shows an amino acid sequence (SEQ ID No. 1) a lipid acyl transferase from Aeromonas hydrophila (ATCC #7965);

FIG. 3 shows a pfam00657 consensus sequence from database version 6 (SEQ ID No. 2);

FIG. 4 shows an amino acid sequence (SEQ ID No. 3) obtained from the organism Aeromonas hydrophila (P10480; GI:121051);

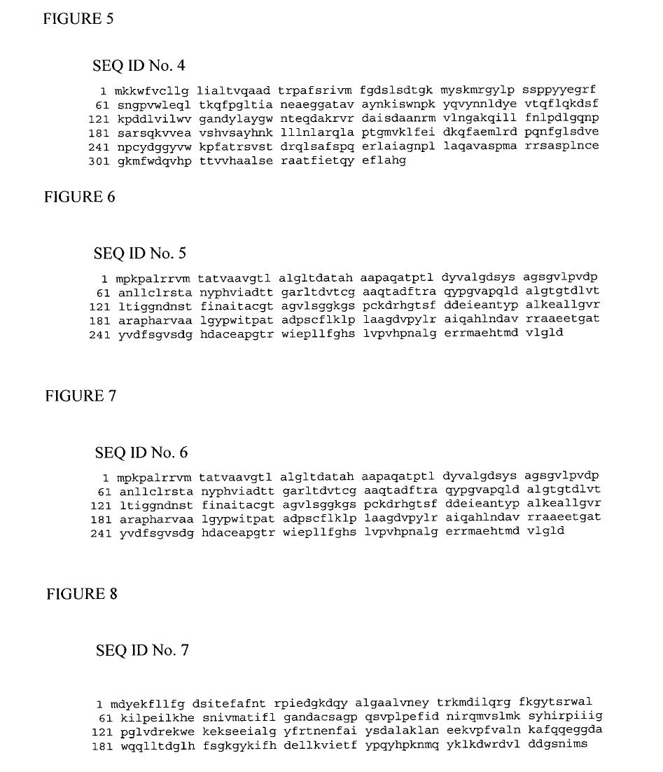

FIG. 5 shows an amino acid sequence (SEQ ID No. 4) obtained from the organism Aeromonas salmonicida (AAG098404; GI:9964017);

FIG. 6 shows an amino acid sequence (SEQ ID No. 5) obtained from the organism Streptomyces coelicolor A3(2) (Genbank accession number NP_{—}631558);

FIG. 7 shows an amino acid sequence (SEQ ID No. 6) obtained from the organism Streptomyces coelicolor A3(2) (Genbank accession number: CAC42140);

FIG. 8 shows an amino acid sequence (SEQ ID No. 7) obtained from the organism Saccharomyces cerevisiae (Genbank accession number P41734);

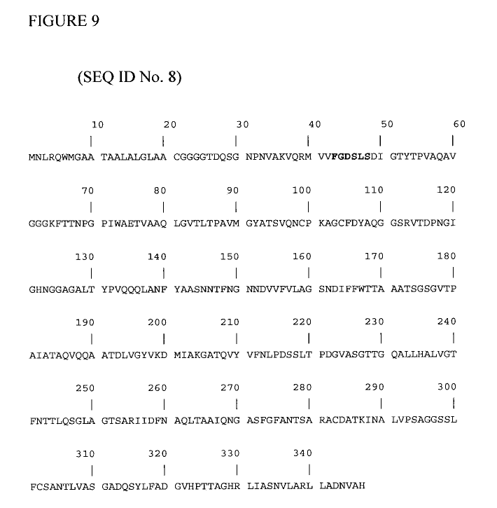

FIG. 9 shows an amino acid sequence (SEQ ID No. 8) obtained from the organism Ralstonia (Genbank accession number: AL646052);

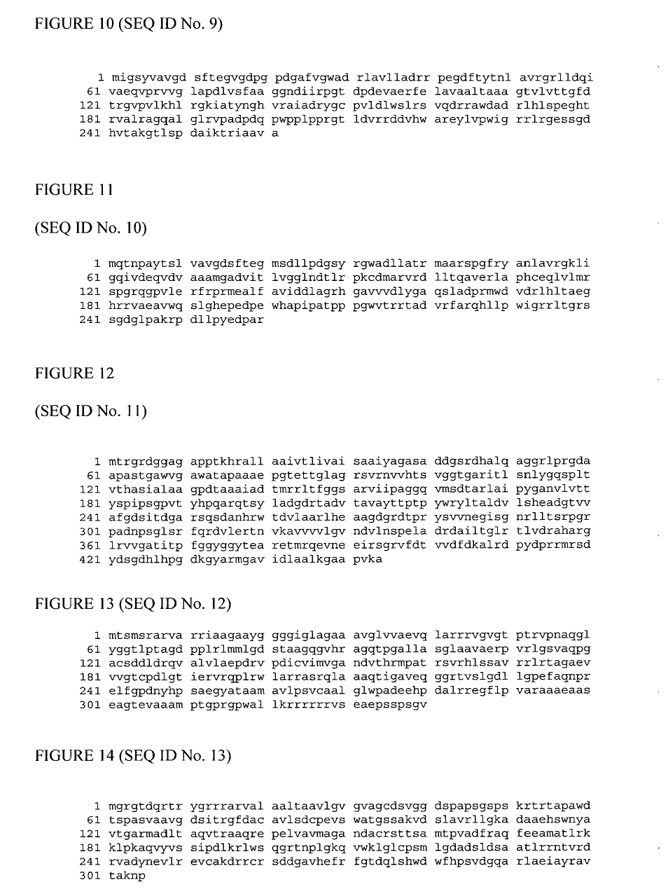

FIG. 10 shows SEQ ID No. 9. Scoe1 NCBI protein accession code CAB39707.1 GI:4539178 conserved hypothetical protein [Streptomyces coelicolor A3(2)];

FIG. 11 shows an amino acid shown as SEQ ID No. 10. Scoe2 NCBI protein accession code CAC01477.1 GI:9716139 conserved hypothetical protein [Streptomyces coelicolor A3(2)];

FIG. 12 shows an amino acid sequence (SEQ ID No. 11) Scoe3 NCBI protein accession code CAB88833.1 GI:7635996 putative secreted protein. [Streptomyces coelicolor A3(2)];

FIG. 13 shows an amino acid sequence (SEQ ID No. 12) Scoe4 NCBI protein accession code CAB89450.1 GI:7672261 putative secreted protein. [Streptomyces coelicolor A3(2)];

FIG. 14 shows an amino acid sequence (SEQ ID No. 13) Scoe5 NCBI protein accession code CAB62724.1 GI:6562793 putative lipoprotein [Streptomyces coelicolor A3(2)];

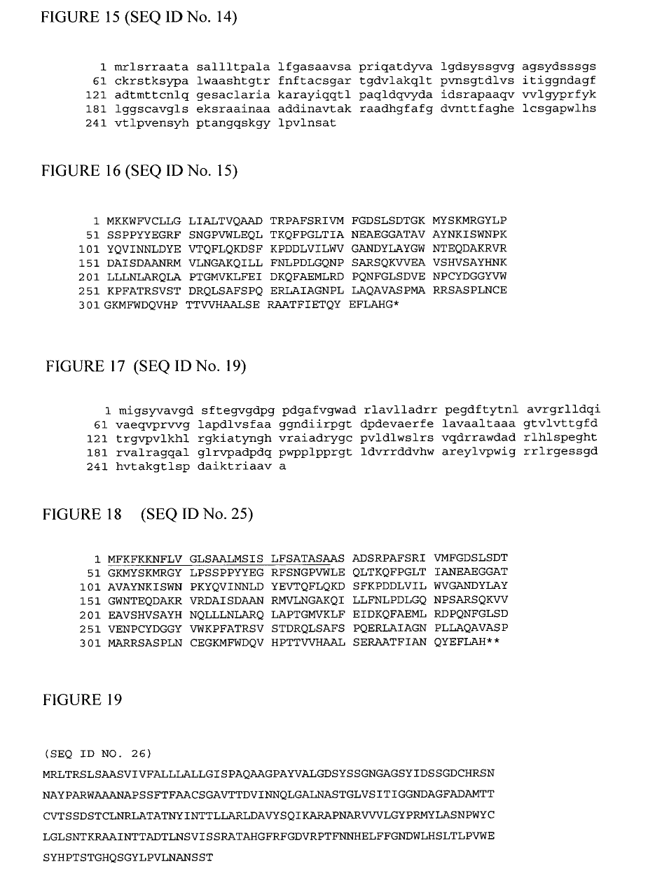

FIG. 15 shows an amino acid sequence (SEQ ID No. 14) Srim1 NCBI protein accession code AAK84028.1 GI:15082088 GDSL-lipase [Streptomyces rimosus];

FIG. 16 shows an amino acid sequence (SEQ ID No. 15) of a lipid acyltransferase from Aeromonas salmonicida subsp. Salmonicida (ATCC#14174);

FIG. 17 shows SEQ ID No. 19. Scoe1 NCBI protein accession code CAB39707.1 GI:4539178 conserved hypothetical protein [Streptomyces coelicolor A3(2)];

FIG. 18 shows an amino acid sequence (SEQ ID No. 25) of the fusion construct used for mutagenesis of the Aeromonas hydrophila lipid acyltransferase gene. The underlined amino acids is a xylanase signal peptide;

FIG. 19 shows a polypeptide sequence of a lipid acyltransferase enzyme from Streptomyces (SEQ ID No. 26);

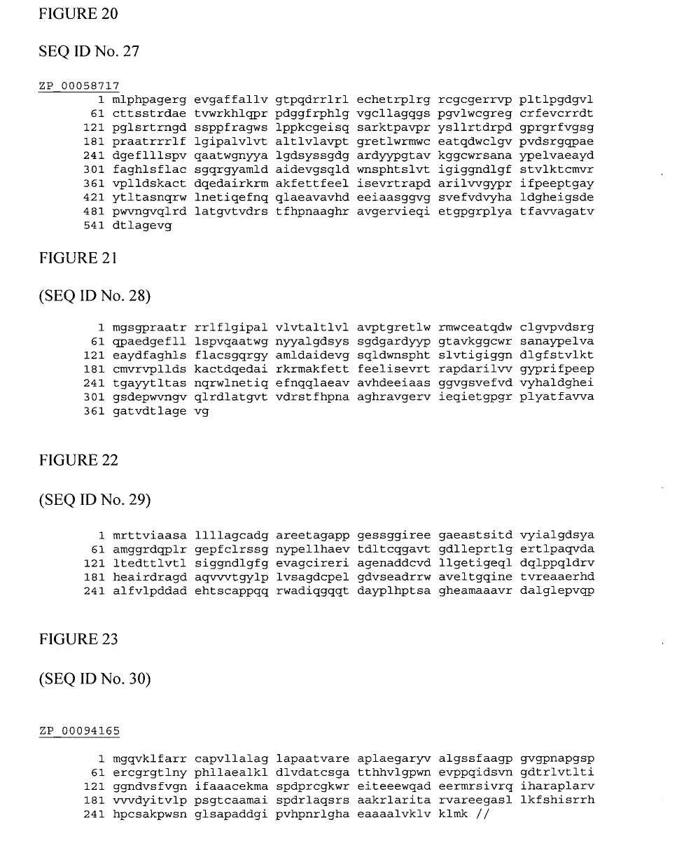

FIG. 20 shows a polypeptide sequence of a lipid acyltransferase enzyme from Thermobifida (SEQ ID No. 27);

FIG. 21 shows a polypeptide sequence of a lipid acyltransferase enzyme from Thermobifida (SEQ ID No. 28);

FIG. 22 shows a polypeptide of a lipid acyltransferase enzyme from Corynebacterium efficiens GDSx 300 amino acid (SEQ ID No. 29);

FIG. 23 shows a polypeptide of a lipid acyltransferase enzyme from Novosphingobium aromaticivorans GDSx 284 amino acid (SEQ ID No. 30);

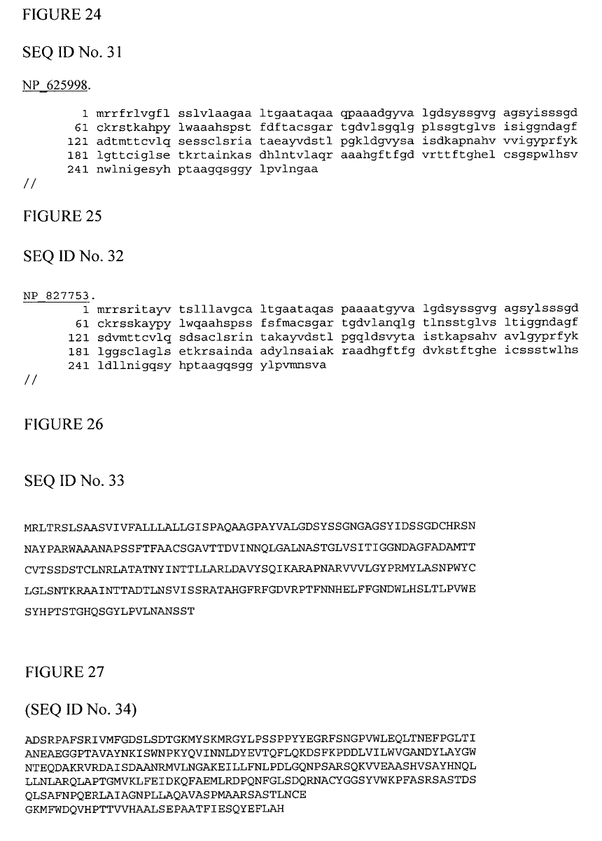

FIG. 24 shows a polypeptide of a lipid acyltransferase enzyme from Streptomyces coelicolor GDSx 269 aa (SEQ ID No. 31);

FIG. 25 shows a polypeptide of a lipid acyltransferase enzyme from Streptomyces avermitilis\GDSx 269 amino acid (SEQ ID No. 32);

FIG. 26 shows a polypeptide of a lipid acyltransferase enzyme from Streptomyces (SEQ ID No. 33);

FIG. 27 shows an amino acid sequence (SEQ ID No. 34) obtained from the organism Aeromonas hydrophila (P10480; GI:121051) (notably, this is the mature sequence);

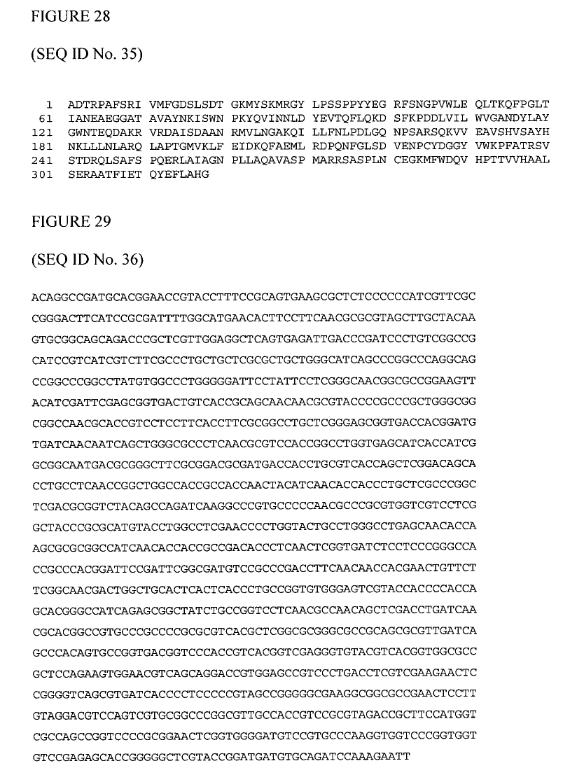

FIG. 28 shows the amino acid sequence (SEQ ID No. 35) of a mutant Aeromonas salmonicida mature lipid acyltransferase (GCAT) (notably, this is the mature sequence);

FIG. 29 shows a nucleotide sequence (SEQ ID No. 36) from Streptomyces thermosacchari;

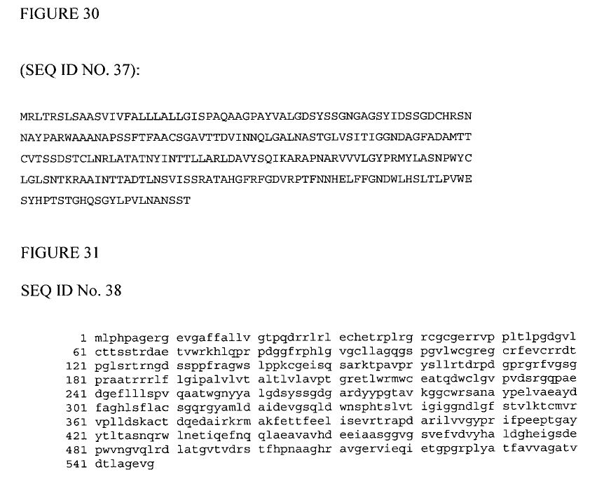

FIG. 30 shows an amino acid sequence (SEQ ID No. 37) from Streptomyces thermosacchari;

FIG. 31 shows an amino acid sequence (SEQ ID No. 38) from Thermobifida fusca/GDSx 548 amino acid;

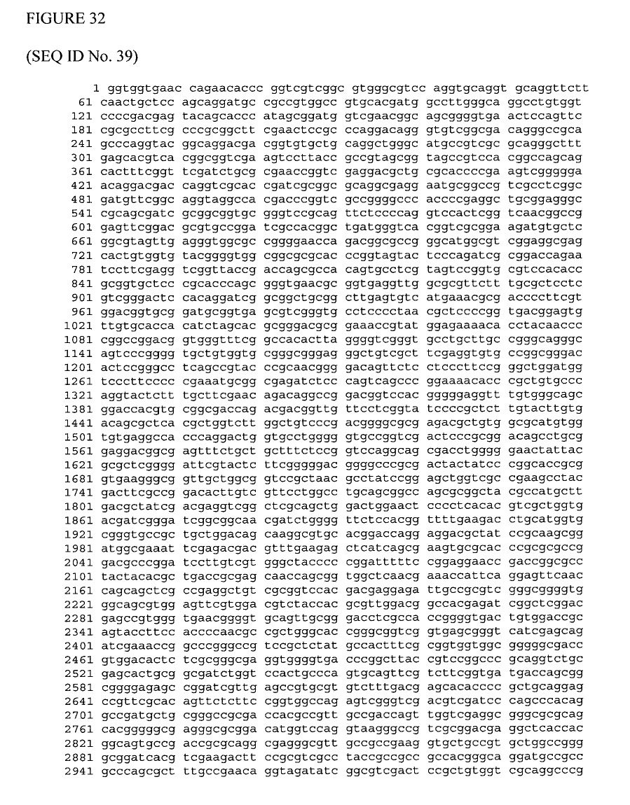

FIG. 32 shows a nucleotide sequence (SEQ ID No. 39) from Thermobifida fusca;

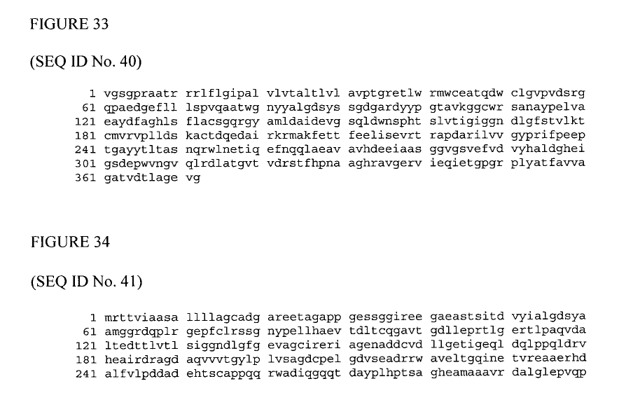

FIG. 33 shows an amino acid sequence (SEQ ID No. 40) from Thermobifida fusca/GDSx;

FIG. 34 shows an amino acid sequence (SEQ ID No. 41) from Corynebacterium efficiens/GDSx 300 amino acid;

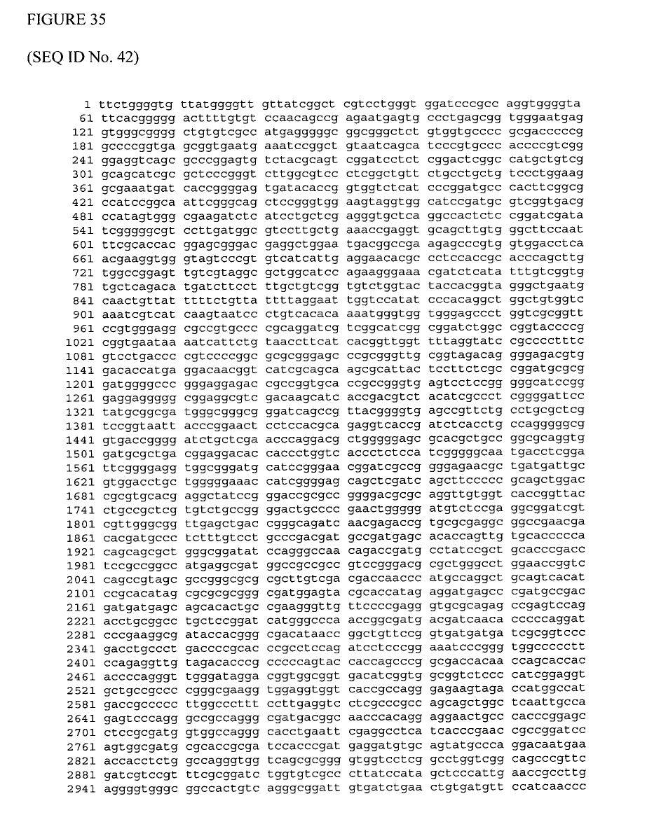

FIG. 35 shows a nucleotide sequence (SEQ ID No. 42) from Corynebacterium efficiens;

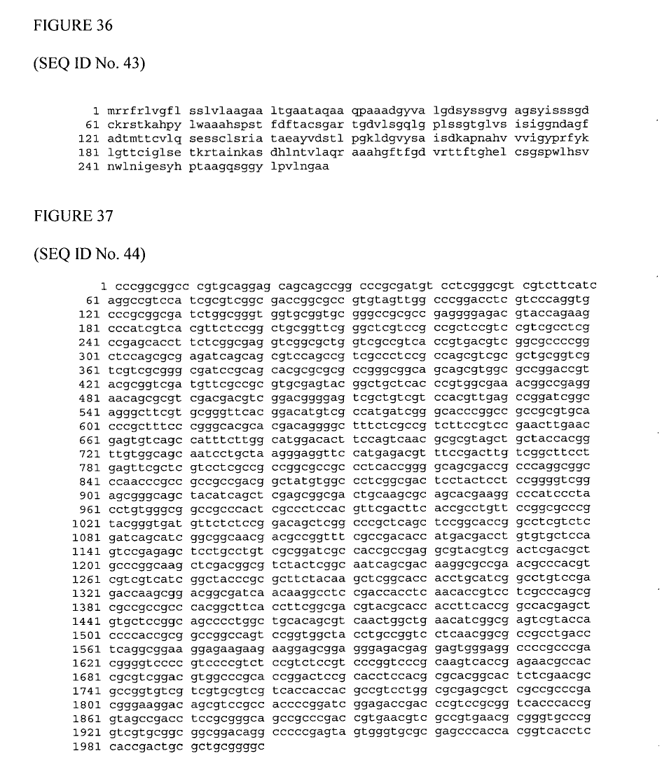

FIG. 36 shows an amino acid sequence (SEQ ID No. 43) from S. coelicolor/GDSx 268 amino acid;

FIG. 37 shows a nucleotide sequence (SEQ ID No. 44) from S. coelicolor;

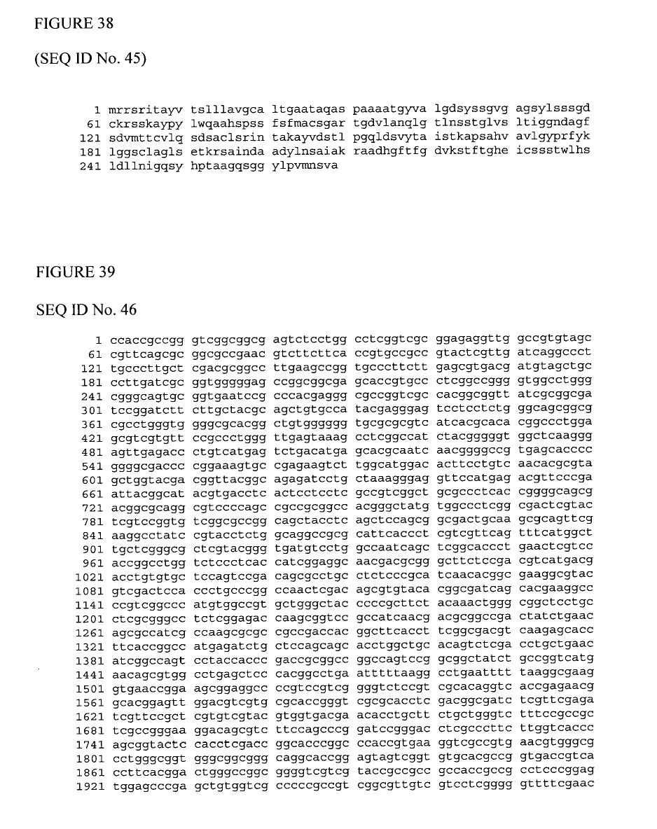

FIG. 38 shows an amino acid sequence (SEQ ID No. 45) from S. avermitilis;

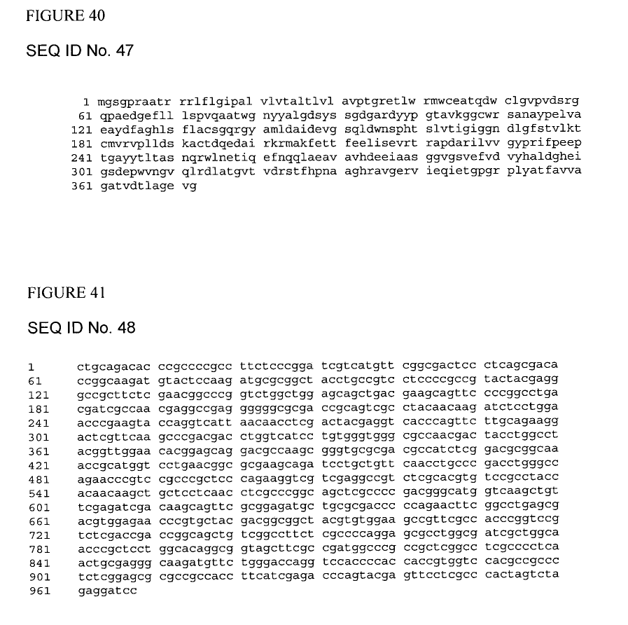

FIG. 39 shows a nucleotide sequence (SEQ ID No. 46) from S. avermitilis;

FIG. 40 shows an amino acid sequence (SEQ ID No. 47) from Thermobifida fusca/GDSx;

FIG. 41 shows a nucleotide sequence (SEQ ID No. 48) from Thermobifida fusca/GDSx;

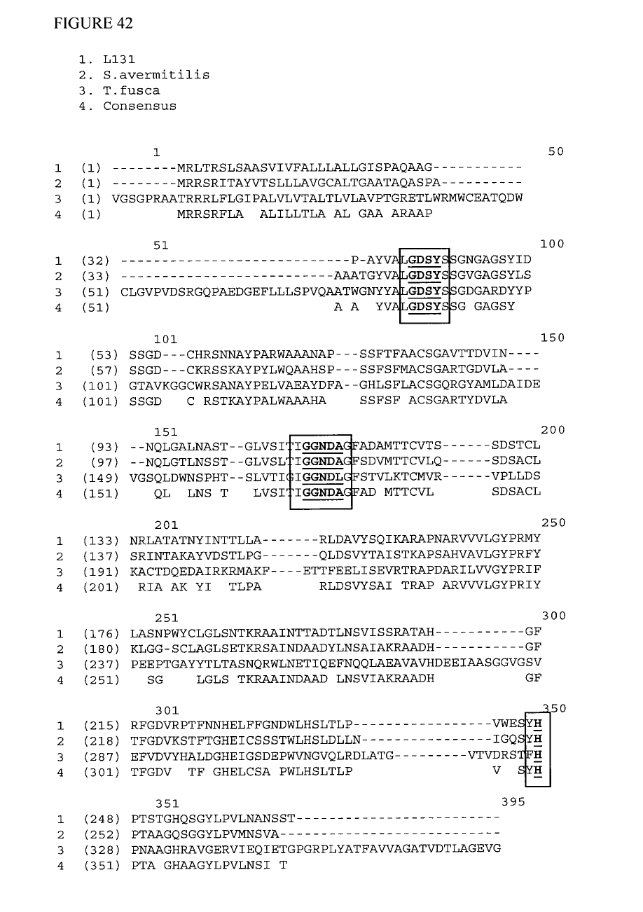

FIG. 42 shows an alignment of the L131 and homologues from S. avermitilis and T. fusca illustrates that the conservation of the GDSx motif (GDSY in L131 and S. avermitilis and T. fusca), the GANDY box, which is either GGNDA or GGNDL, and the HPT block (considered to be the conserved catalytic histidine). These three conserved blocks are highlighted;

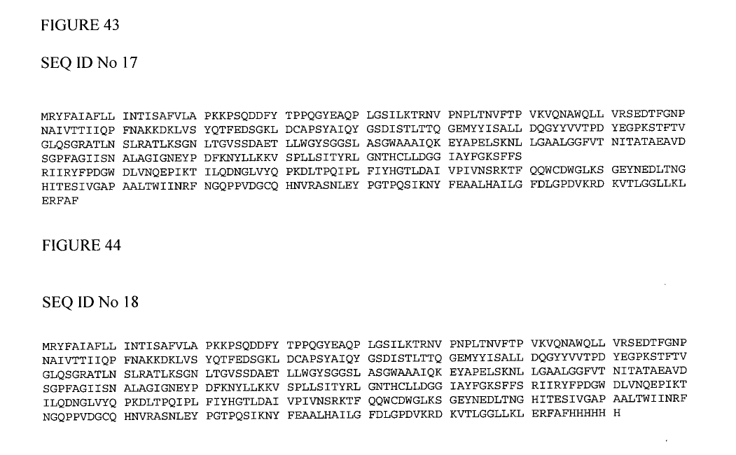

FIG. 43 shows SEQ ID No 17 which is the amino acid sequence of a lipid acyltransferase from Candida parapsilosis;

FIG. 44 shows SEQ ID No 18 which is the amino acid sequence of a lipid acyltransferase from Candida parapsilosis;

FIG. 45 shows a nucleotide sequence from Aeromonas salmonicida (SEQ ID No. 49) including the signal sequence (preLAT—positions 1 to 87);

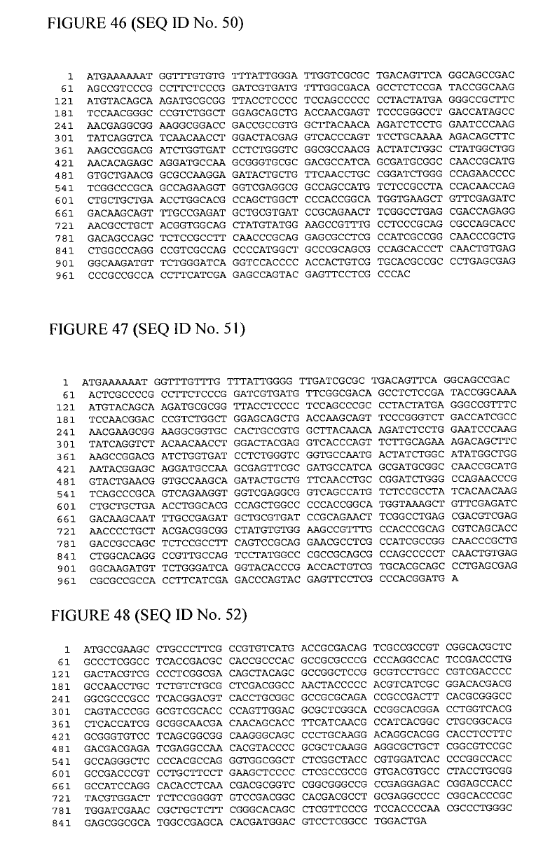

FIG. 46 shows a nucleotide sequence (SEQ ID No. 50) encoding a lipid acyl transferase according to the present invention obtained from the organism Aeromonas hydrophila;

FIG. 47 shows a nucleotide sequence (SEQ ID No. 51) encoding a lipid acyl transferase according to the present invention obtained from the organism Aeromonas salmonicida;

FIG. 48 shows a nucleotide sequence (SEQ ID No. 52) encoding a lipid acyl transferase according to the present invention obtained from the organism Streptomyces coelicolor A3(2) (Genbank accession number NC_{—}003888.1:8327480.8328367);

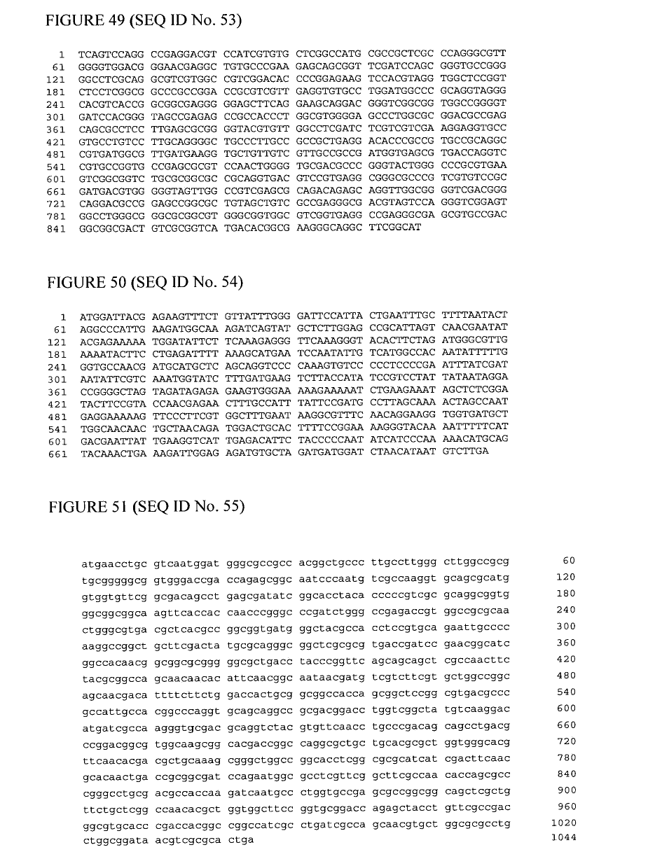

FIG. 49 shows a nucleotide sequence (SEQ ID No. 53) encoding a lipid acyl transferase according to the present invention obtained from the organism Streptomyces coelicolor A3(2) (Genbank accession number AL939131.1:265480.266367);

FIG. 50 shows a nucleotide sequence (SEQ ID No. 54) encoding a lipid acyl transferase according to the present invention obtained from the organism Saccharomyces cerevisiae (Genbank accession number Z75034);

FIG. 51 shows a nucleotide sequence (SEQ ID No. 55) encoding a lipid acyl transferase according to the present invention obtained from the organism Ralstonia;

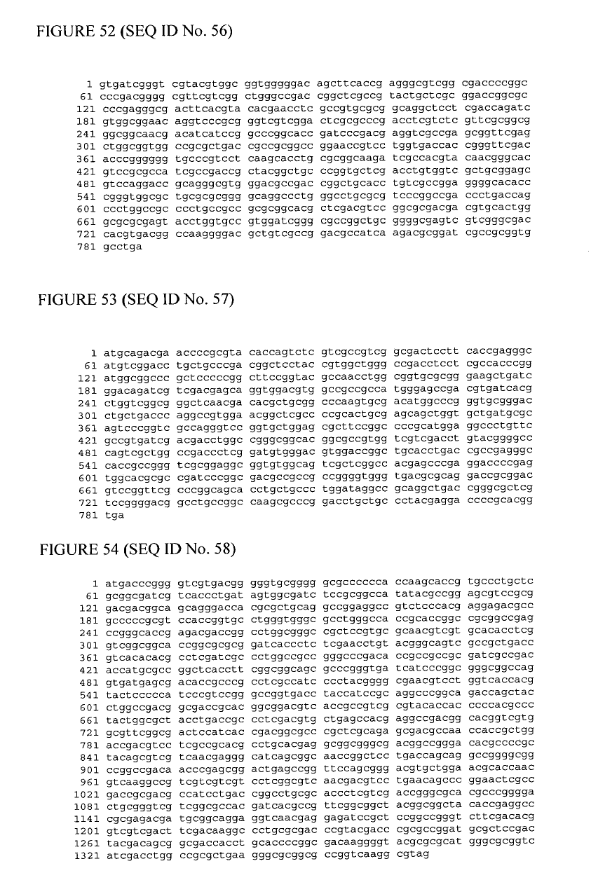

FIG. 52 shows a nucleotide sequence shown as SEQ ID No. 56 encoding NCBI protein accession code CAB39707.1 GI:4539178 conserved hypothetical protein [Streptomyces coelicolor A3 (2)];

FIG. 53 shows a nucleotide sequence shown as SEQ ID No. 57 encoding Scoe2 NCBI protein accession code CAC01477.1 GI:9716139 conserved hypothetical protein [Streptomyces coelicolor A3(2)];

FIG. 54 shows a nucleotide sequence shown as SEQ ID No. 58 encoding Scoe3 NCBI protein accession code CAB88833.1 GI:7635996 putative secreted protein. [Streptomyces coelicolor A3(2)];

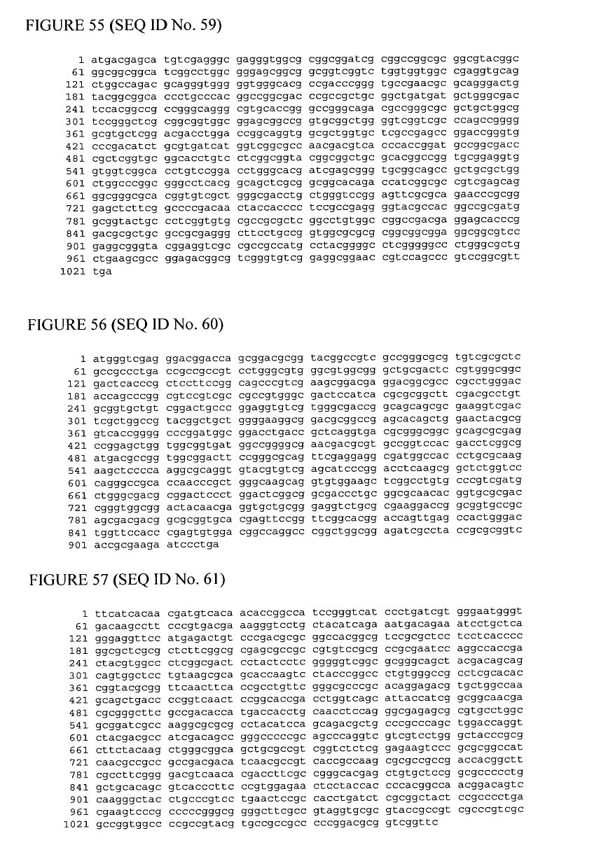

FIG. 55 shows a nucleotide sequence shown as SEQ ID No. 59 encoding Scoe4 NCBI protein accession code CAB89450.1 GI:7672261 putative secreted protein. [Streptomyces coelicolor A3(2)];

FIG. 56 shows a nucleotide sequence shown as SEQ ID No. 60, encoding Scoe5 NCBI protein accession code CAB62724.1 GI:6562793 putative lipoprotein [Streptomyces coelicolor A3(2)];

FIG. 57 shows a nucleotide sequence shown as SEQ ID No. 61 encoding Srim1 NCBI protein accession code AAK84028.1 GI:15082088 GDSL-lipase [Streptomyces rimosus];

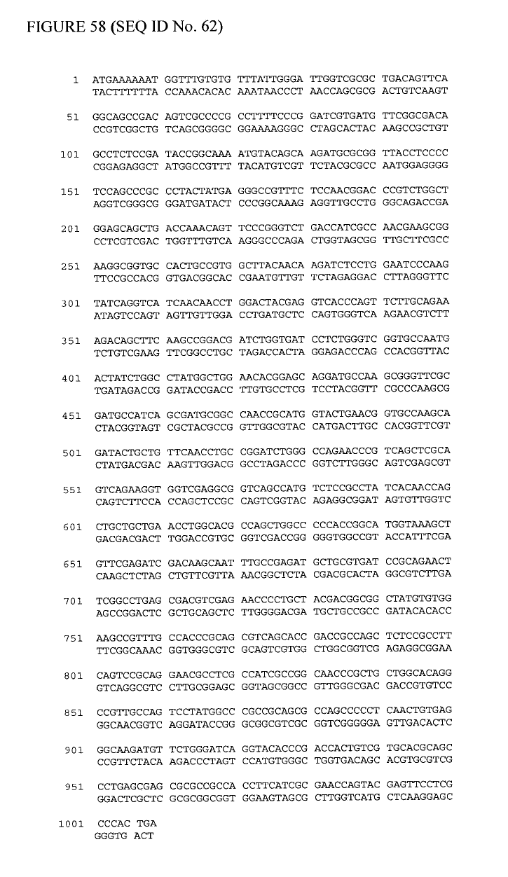

FIG. 58 shows a nucleotide sequence (SEQ ID No. 62) encoding a lipid acyltransferase from Aeromonas hydrophila (ATCC #7965);

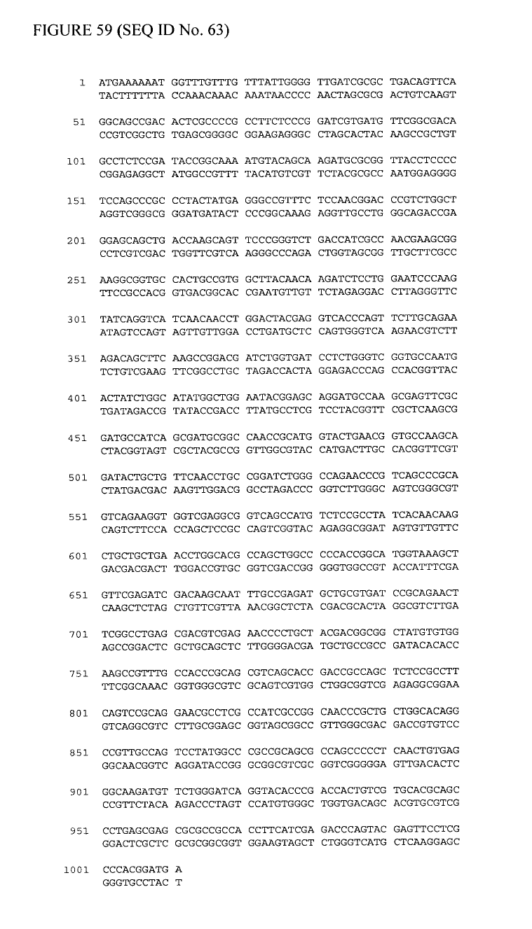

FIG. 59 shows a nucleotide sequence (SEQ ID No 63) encoding a lipid acyltransferase from Aeromonas salmonicida subsp. Salmonicida (ATCC#14174);

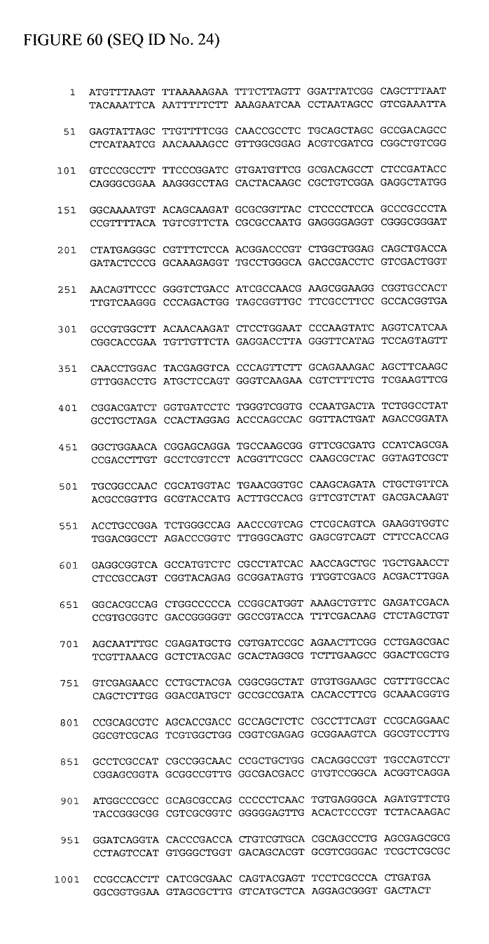

FIG. 60 shows a nucleotide sequence (SEQ ID No. 24) encoding an enzyme from Aeromonas hydrophila including a xylanase signal peptide;

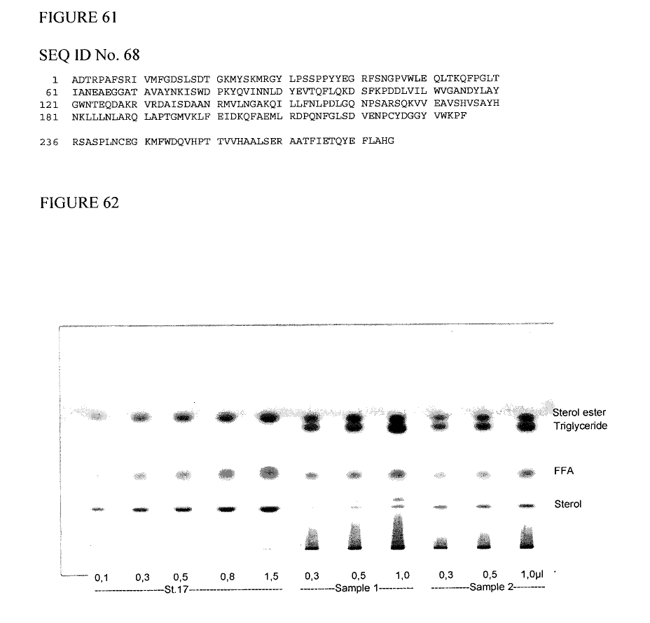

FIG. 61 shows the amino acid sequence (SEQ ID No. 68) of a mutant Aeromonas salmonicida mature lipid acyltransferase (GCAT) with a mutation of Asn80Asp (notably, amino acid 80 is in the mature sequence) and after undergoing post-translational modification—amino acid residues 235 and 236 of SEQ ID No. 68 are not covalently linked following post-translational modification. The two peptides formed are held together by one or more S-S bridges. Amino acid 236 in SEQ ID No. 68 corresponds with the amino acid residue number 274 in SEQ ID No. 16 shown herein.

FIG. 62 shows a TLC analysis of sterol gum phase reaction products

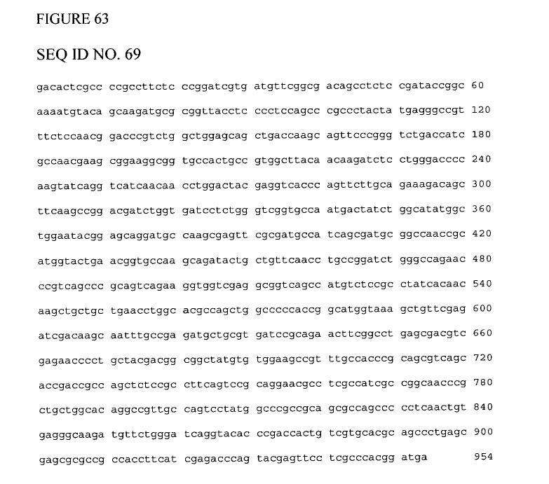

FIG. 63 shows a nucleotide sequence (SEQ ID NO. 69) which encodes a lipid acyltransferase from A. salmonicida;

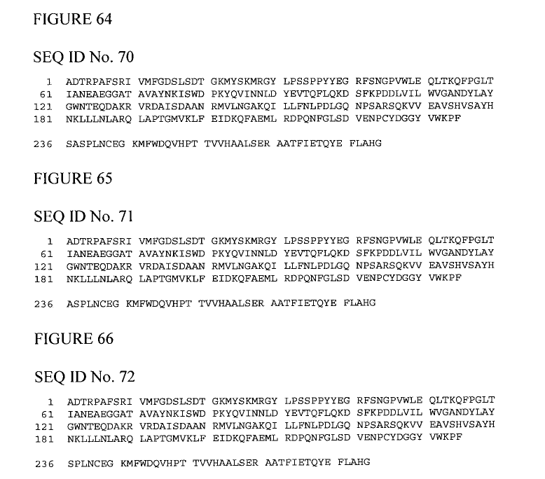

FIG. 64 shows the amino acid sequence of a mutant Aeromonas salmonicida mature lipid acyltransferase (GCAT) with a mutation of Asn80Asp (notably, amino acid 80 is in the mature sequence)—shown herein as SEQ ID No. 16—and after undergoing post-translational modification as SEQ ID No. 70—amino acid residues 235 and 236 of SEQ ID No. 70 are not covalently linked following post-translational modification; the two peptides formed are held together by one or more S-S bridges; amino acid 236 in SEQ ID No. 70 corresponds with the amino acid residue number 275 in SEQ ID No. 16 shown herein;

FIG. 65 shows the amino acid sequence of a mutant Aeromonas salmonicida mature lipid acyltransferase (GCAT) with a mutation of Asn80Asp (notably, amino acid 80 is in the mature sequence)—shown herein as SEQ ID No. 16—and after undergoing post-translational modification as SEQ ID No. 71—amino acid residues 235 and 236 of SEQ ID No. 71 are not covalently linked following post-translational modification; the two peptides formed are held together by one or more S-S bridges; amino acid 236 in SEQ ID No. 71 corresponds with the amino acid residue number 276 in SEQ ID No. 16 shown herein; and

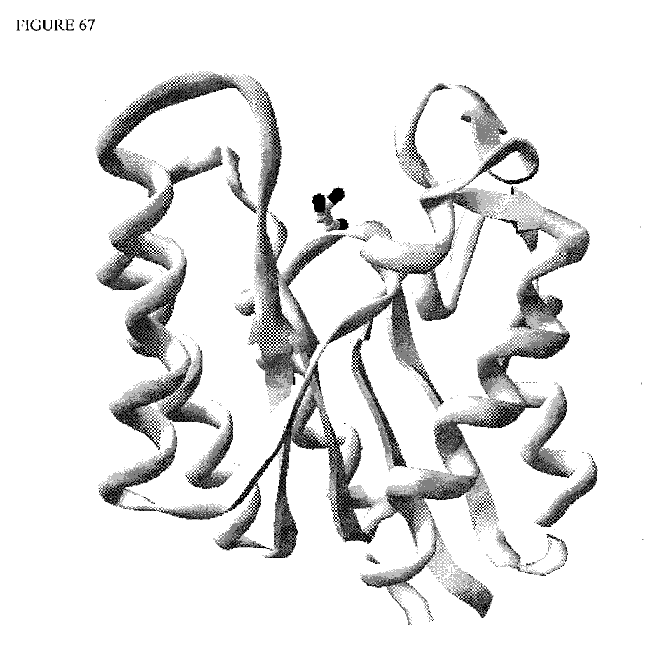

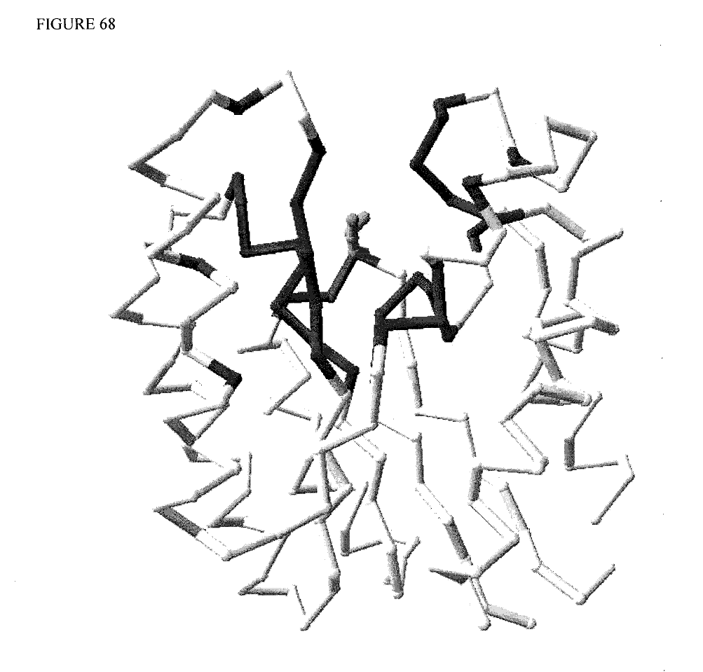

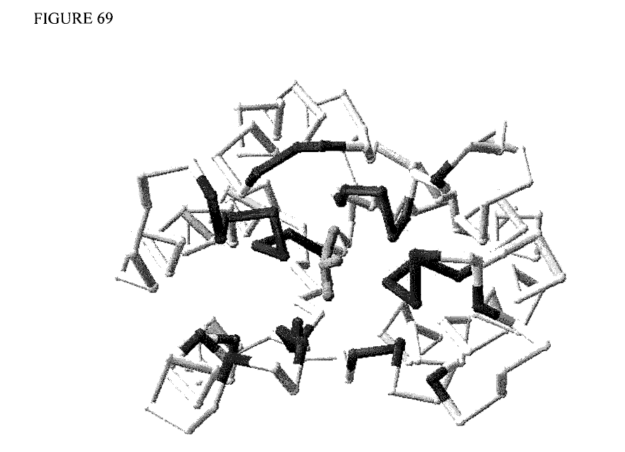

FIG. 66 shows the amino acid sequence of a mutant Aeromonas salmonicida mature lipid acyltransferase (GCAT) with a mutation of Asn80Asp (notably, amino acid 80 is in the mature sequence)—shown herein as SEQ ID No. 16—and after undergoing post-translational modification as SEQ ID No. 72—amino acid residues 235 and 236 of SEQ ID No. 72 are not covalently linked following post-translational modification; the two peptides formed are held together by one or more S-S bridges; amino acid 236 in SEQ ID No. 72 corresponds with the amino acid residue number 277 in SEQ ID No. 16 shown herein.

FIG. 67 shows a ribbon representation of the 1IVN.PDB crystal structure which has glycerol in the active site. The Figure was made using the Deep View Swiss-PDB viewer;

FIG. 68 shows 1IVN.PDB Crystal Structure—Side View using Deep View Swiss-PDB viewer, with glycerol in active site—residues within 10 \acute{Å} of active site glycerol are coloured black;

FIG. 69 shows 1IVN.PDB Crystal Structure—Top View using Deep View Swiss-PDB viewer, with glycerol in active site—residues within 10 \acute{Å} of active site glycerol are coloured black;

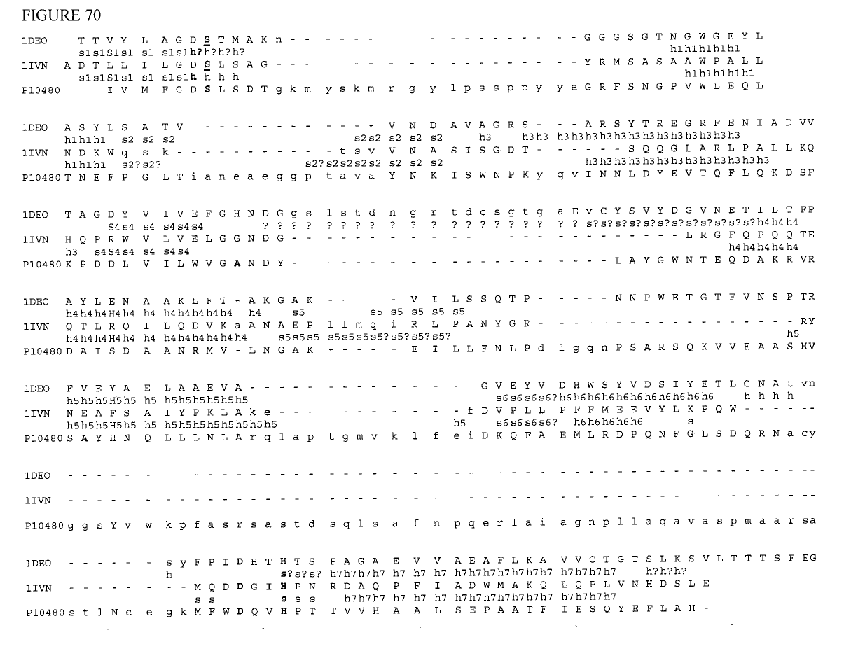

FIG. 70 shows alignment 1;

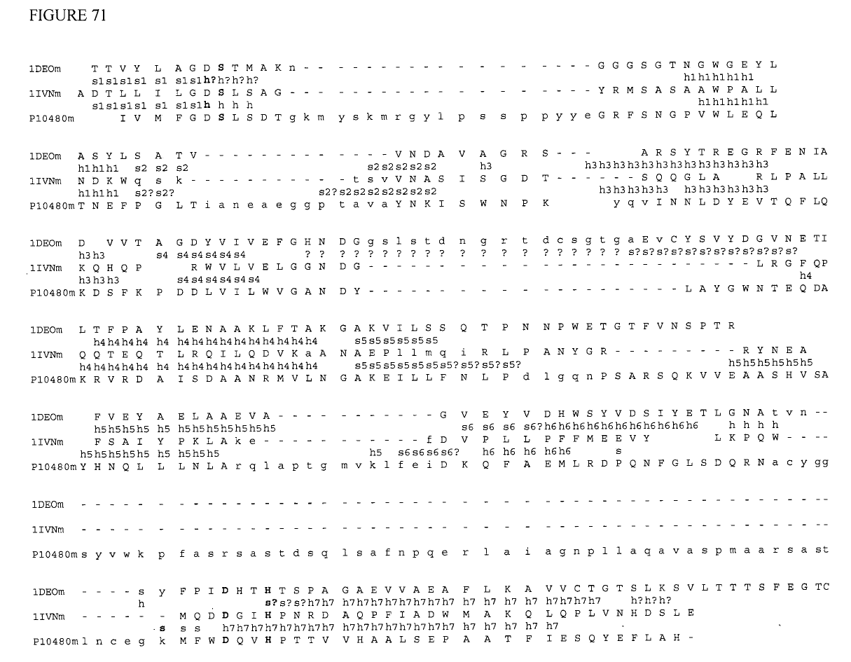

FIG. 71 shows alignment 2;

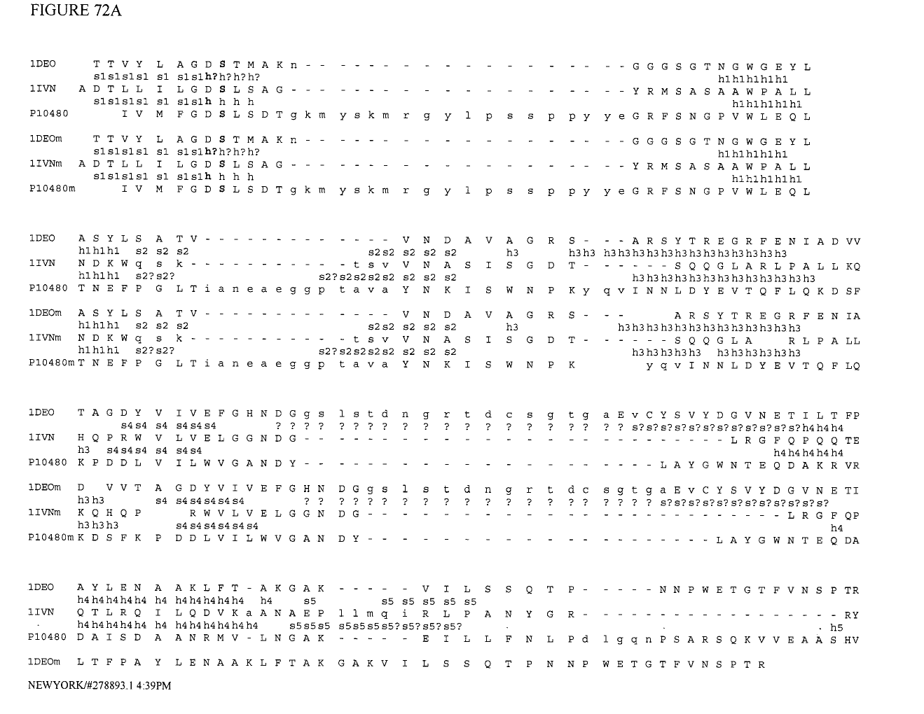

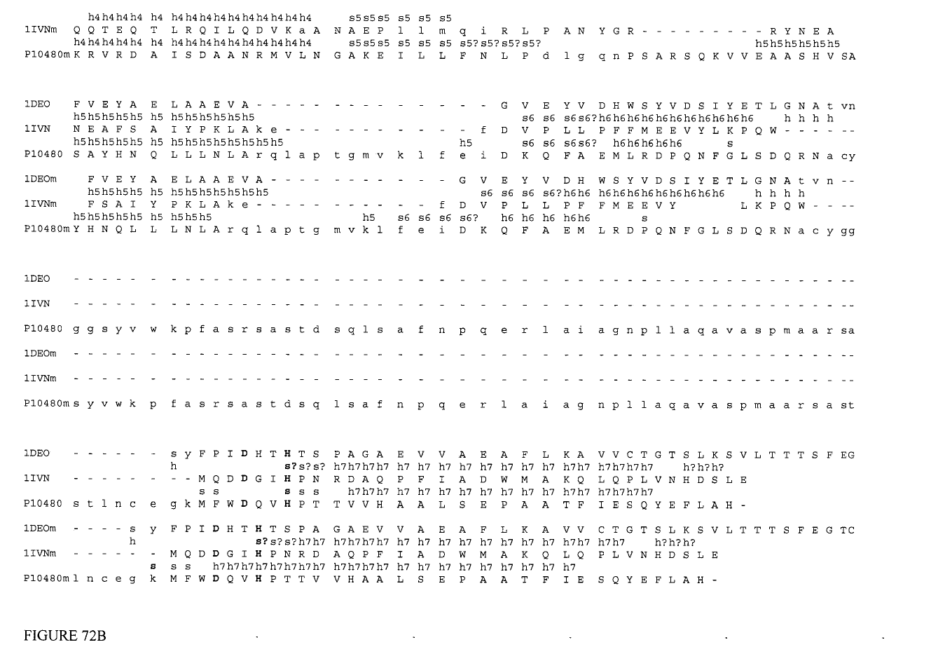

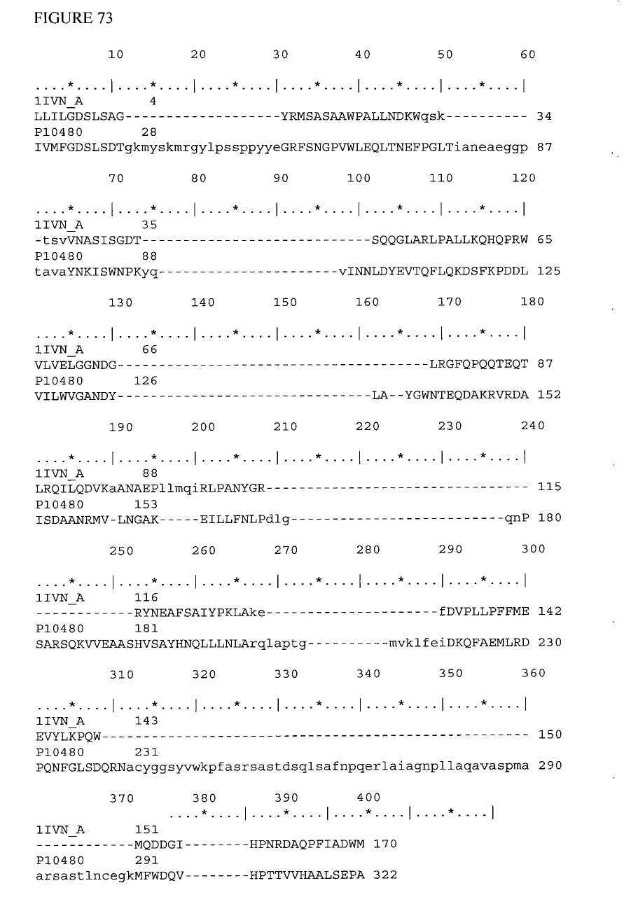

FIGS. 72A, 72B and 73 show an alignment of 1IVN to P10480 (P10480 is the database sequence for A. hydrophila enzyme), this alignment was obtained from the PFAM database and used in the model building process; and

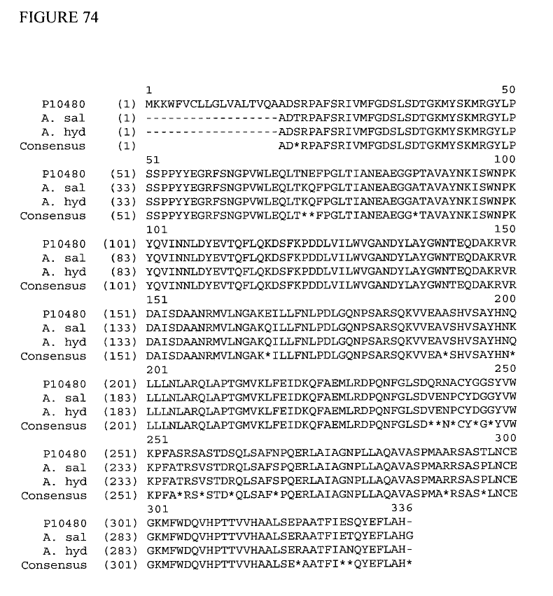

FIG. 74 shows an alignment where P10480 is the database sequence for Aeromonas hydrophila. This sequence is used for the model construction and the site selection (note that the full protein (SEQ ID No. 25) is depicted, the mature protein (equivalent to SEQ ID No. 34) starts at residue 19. A. sal is Aeromonas salmonicida (SEQ ID No. 4) GDSX lipase, A. hyd is Aeromonas hydrophila (SEQ ID No. 34) GDSX lipase; the consensus sequence contains a * at the position of a difference between the listed sequences).

### EXAMPLE 1

Phytosterol esters and phytostanol esters have found several application in industry, including in the food industry as a functional ingredient with cholesterol lowering effects.

Synthesis of phytosterol esters and phytostanol esters by chemical catalysis is quite complicated, if often carried out using organic solvents and often needs several purification steps to isolate the ester formed.

The inventors have found that lipid acyltransferases can be used as an enzymatic catalyst for the synthesis of phytosterol ester from phytosterol and phytostanol ester from phytostanol.

The lipid donor is a phospholipid composition. Suitably the phospholipid composition may be a gum phase obtained from water degumming of soya oil. Preferably the phytosterol ester and/or phytostanol ester is isolated or purified from the reaction composition or admixture and used as an isolated phytosterol ester and/or phytostanol ester. Notably however, the reaction composition or admixture does not typically comprise harmful constituents (such as organic solvents and the like) and therefore the need for complex purification and/or isolation of the phytosterol esters or phytostanol esters can be avoided.

### Material and Methods:

KLM3′-Glycerophospholipid cholesterol acyltransferase (FoodPro LysoMax Oil) (KTP 08015)—Activity 1300 LATU/g (available from Danisco A/S)
Gum phase from water degumming of Brazilian soya bean (called SYP from Solae Aarhus)
Dried gum phase, SYP dried on a rotary evaporator.
Phytosterol-Generol 122 N from Henkel Germany

### HPTLC Analysis

The phytosterol and phytosterol ester samples were analysed using HPTLC.

### Applicator: Automatic TLC Sampler 4, CAMAG

HPTLC plate: 20×10 cm, Merck no. 1.05641. Activated 10 minutes at 160° C. before use.

Application:
0.2 g reaction mixture of gum and phytosterol was dissolved in 3 ml Hexan:Isopropanol 3:2.
0.3 or 0.5 or 1 μl of the sample was applied to the HPTLC plate.
A standard solution (no. 17) containing 0.1% oleic acid, 0.1% cholesterol and 0.1% cholesterol ester was applied (0.1, 0.3, 0.5, 0.8 and 1.5 μl) and used for the calculation of the phytosterol and phytosterol ester in the reaction mixture.

### TLC Applicator.

Running buffer no. 5: P-ether:Methyl Tert Butyl Ketone:Acetic acid 70:30:1

Elution: The plate was eluted 7 cm using an Automatic Developing Chamber ADC2 from Camag.

### Development:

The plate was dried on a Camag TLC Plate Heater III for 6 minutes at 160° C., cooled, and dipped into 6% cupri acetate in 16% H₃PO₄. Additionally dried 10 minutes at 160° C. and evaluated directly.

The density of the components on the TLC plate was analysed by a Camag TLC Scanner 3.

### EXPERIMENTAL

Enzymatic synthesis of phytosterol ester was made with the recipes shown in Table 1

TABLE 1
Recipe for synthesis of sterol ester

| | | Sample 1 | Sample 2 |
|---|---|---|---|
| | | (reaction | (reaction |
| | | composition) | composition) |
| Dried gum phase | g | 10 | |
| Gum phase (comprising 30.3% | g | | 15 |
| water, 41.8% phospholipids and | | | |
| 27.9% triglyceride and fatty acids) | | | |
| Generol 122N | g | 1 | 1 |
| KLM3′, 1300 TIPU/g | g | 0.1 | 0.1 |
| Water | g | 0.2 | |

Each of the gum phases and Generol 122 N were mixed together. In sample 1 most of the phytosterols were dissolved. In sample 2 the phytosterols were only partly solubilised. The enzyme (and water if added) were added and the samples were incubated at 55° C. and samples were taken out after 1 and 4 days. After 4 days sample 1 was a homogenous liquid with no phytosterol. Sample 2 was also almost homogenous but the sample was not liquid.

The overall water content in the reaction mixture of sample 1 was about 2.2% w/w water, and the overall water content in the reaction mixture of sample 2 was about 28.5% w/w water.

The samples were analysed by TLC and the conversion of phytosterols were calculated with results shown in table 2 and FIG. 62.

TABLE 2
% phytosterol esterified as a function of reaction time.

| | Reaction time | Esterified |
|---|---|---|
| Sample | Days | Sterol, % |
| 1 | 1 | 64.6 |
| 1 | 4 | 94.3 |
| 2 | 1 | 58.6 |
| 2 | 4 | 72.8 |

FIG. 62 shows a TLC analysis of phytosterol gum phase reaction products.

The results in table 2 confirm that lipid acyltransferases (e.g. KLM3′) gives a very high conversion of phytosterol to phytosterol ester in both samples. A>90% conversion was observed in sample 1 and the product appears as a homogenous liquid product with all sterol ester solubilised. A good conversion of phytosterol to phytosterol ester was also observed in sample 2.

By suitable adjustment of the enzyme dosage it is possible have even higher conversion and a shorter incubation time.

The sterol ester may be isolated or purified using any conventional isolation or purification methods. The sterol ester may then be used in food compositions or foodstuffs or personal care products as known in the art.

In some embodiments heat treatment to 100° C. can be used to inactivate the enzyme and the sterol ester phospholipid sample can be used directly in food applications or personal care products for sterol enrichment (i.e. without any isolation or purification).

### Conclusion:

Experiments have shown that it is possible produce phytosterol ester from phytosterols and a phospholipid composition (e.g. a gum phase obtained from water degumming of oil), by an enzymatic reaction catalysed by a lipid acyltransferase. More than 90% conversion of the phytosterol to phytosterol esters is possible.

### EXAMPLE 2

| Recipe | | 1 | 2 | 3 |
|---|---|---|---|---|
| Gum phase (comprising 30.3% | g | 15 | 15 | 15 |
| water, 41.8% phospholipids and | | | | |
| 27.9% triglyceride and fatty acids) | | | | |
| Phytostanol | g | 1 | 1 | 2 |
| KLM3′ (lipid acyltransferase), | g | 0.1 | | 0.1 |
| 1300 TIPU/g | | | | |

Gum phase from water degumming is heated to 55° C. Plant stanol isolated from wood is added during agitation. A lipid acyltransferase (KLM3′) is added and the reaction mixture is incubated at 55° C. with agitation. After 20 hours the reaction mixture is heated to 95° C. to inactivate the enzyme, and the sample is analyzed by HPTLC for stanol and stanol ester.

In sample no 1 and 3 more than 50% of the stanols are esterified and in sample no 2 no stanol esters are formed.

All publications mentioned in the above specification are herein incorporated by reference. Various modifications and variations of the described methods and system of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in biochemistry and biotechnology or related fields are intended to be within the scope of the following claims.

1. A method of producing a phytosterol ester and/or a phytostanol ester comprising:
a) preparing a reaction composition by admixing a phospholipid composition comprising at least between about 10% to about 70% plant phospholipid; a lipid acyltransferase;
and a phytosterol and/or a phytostanol; and optionally water, wherein the reaction composition comprises at least 2% water w/w; and
b) isolating or purifying at least one phytosterol ester and/or phytostanol ester.

2. A method according to claim 1 wherein the phytosterol and/or phytostanol is added in amount of at least 5% of the overall reaction mixture.

3. A method according to claim 1 wherein the phytosterol ester and/or phytostanol ester is admixed with a foodstuff or food ingredient.

4. A method according to claim 1 wherein the phytosterol ester and/or phytostanol ester is admixed with a pharmaceutical diluent, carrier or excipient or a cosmetic diluent, carrier or excipient.

7. A method according to claim 1 wherein the phytosterol and/or phytostanol comprises one or more of the following structural features:
i) a 3-beta hydroxy group or a 3-alpha hydroxy group; and/or
ii) A:B rings in the cis position or A:B rings in the trans position or C₅-C₆ is unsaturated.

8. A method according claim 1 wherein the phytosterol is one or more of the following selected from the group consisting of: alpha-sitosterol, beta-sitosterol, stigmasterol, ergosterol, campesterol, 5,6-dihydrosterol, brassicasterol, alpha-spinasterol, beta-spinasterol, gamma-spinasterol, deltaspinasterol, fucosterol, dimosterol, ascosterol, serebisterol, episterol, anasterol, hyposterol, chondrillasterol, desmosterol, chalinosterol, poriferasterol, clionasterol, sterol glycosides, and other natural or synthetic isomeric forms and derivatives.

9. A method according to claim 1 wherein a lyso-phospholipid is also produced.

10. A method according to claim 9 wherein the lyso-phospholipid is purified or isolated.

11. A method according to claim 1 wherein the lipid acyltransferase comprises a GDSX motif (SEQ ID NO: 20) and/or a GANDY motif (SEQ ID NO: 113).

12. A method according to claim 1 wherein the lipid acyltransferase is characterised as an enzyme which possesses acyltransferase activity and which comprises the amino acid sequence motif GDSX, (SEQ ID NO: 20) wherein X is one or more of the following amino acid residues L, A, V, I, F, Y, H, Q, T, N, M or S.

13. A method according to claim 1 wherein the lipid acyltransferase when tested using the “Protocol for the determination of % acyltransferase activity” has a transferase activity of at least 15%.

14. A method according to claim 1 wherein the lipid acyltransferase is a polypeptide obtainable by expression of a nucleotide sequence in Bacillus licheniformis.

15. The method according to claim 1 wherein the phospholipid composition is a gum phase obtained by degumming (such as by chemical degumming, enzymatic degumming, total degumming, super degumming, water degumming, or a combination of two or more thereof) of an edible oil or a crude edible oil.

16. The method according to claim 1 wherein the phospholipid composition is a soapstock obtained by treating a crude edible oil or an edible oil with an acid and/or an alkaline (such as sodium hydroxide) and isolating the soapstock fraction.

17. The method according to claim 15 or claim 16 wherein the gum phase or the soapstock is purified, or dried, or solvent fractionated, or a combination of two or more thereof prior to admixing same with the lipid acyltransferase and the phytosterol and/or phytostanol and optionally water.

18. A composition comprising a phytosterol ester and/or a phytostanol ester obtained by the method of claim 1.

19. A foodstuff comprising a phytosterol ester and/or a phytostanol ester obtained by the method of claim 1.

20. A personal care (e.g. cosmetic) composition comprising a phytosterol ester and/or a phytostanol ester obtained by the method of claim 1 and optionally a cosmetic diluent, excipient or carrier.

21. A method of producing a foodstuff comprising a phytosterol ester and/or a phytostanol ester, wherein the method comprises the step of adding the composition of claim 18 to a foodstuff and/or a food material.

22. A method of producing a personal care product (e.g. a cosmetic) comprising a phytosterol ester and/or a phytostanol ester, wherein the method comprises the step of adding the composition of claim 18 to a further personal care product (cosmetic) constituent.


SEQUENCE LISTING (118 sequences)

SEQ ID NO: 1 - protein, 335 aa, Aeromonas hydrophila
MKKWFVCLLGLVALTVQAADSRPAFSRIVMFGDSLSDTGKMYSKMRGYLPSSPPYYEGRFSNGPVWLEQLTKQFPGLTIANEAEGGATAVAYNKISWNPKYQVINNLDYEVTQFLQKDSFKPDDLVILWVGANDYLAYGWNTEQDAKRVRDAISDAANRMVLNGAKQILLFNLPDLGQNPSARSQKVVEAVSHVSAYHNQLLLNLARQLAPTGMVKLFEIDKQFAEMLRDPQNFGLSDVENPCYDGGYVWKPFATRSVSTDRQLSAFSPQERLAIAGNPLLAQAVASPMARRSASPLNCEGKMFWDQVHPTTVVHAALSERAATFIANQYEFLAH

SEQ ID NO: 2 - protein, 361 aa, Artificial Sequence
  Description of Artificial Sequence Synthetic pfam00657 polypeptide
IVAFGDSLTDGEAYYGDSDGGGWGAGLADRLTALLRLRARPRGVDVFNRGISGRTSDGRLIVDALVALLFLAQSLGLPNLPPYLSGDFLRGANFASAGATILPTSGPFLIQVQFKDFKSQVLELRQALGLLQELLRLLPVLDAKSPDLVTIMIGTNDLITSAFFGPKSTESDRNVSVPEFKDNLRQLIKRLRSNNGARIIVLITLVILNLGPLGCLPLKLALALASSKNVDASGCLERLNEAVADFNEALRELAISKLEDQLRKDGLPDVKGADVPYVDLYSIFQDLDGIQNPSAYVYGFETTKACCGYGGRYNYNRVCGNAGLCNVTAKACNPSSYLLSFLFWDGFHPSEKGYKAVAEAL

SEQ ID NO: 3 - protein, 335 aa, Aeromonas hydrophila
MKKWFVCLLGLVALTVQAADSRPAFSRIVMFGDSLSDTGKMYSKMRGYLPSSPPYYEGRFSNGPVWLEQLTNEFPGLTIANEAEGGPTAVAYNKISWNPKYQVINNLDYEVTQFLQKDSFKPDDLVILWVGANDYLAYGWNTEQDAKRVRDAISDAANRMVLNGAKEILLFNLPDLGQNPSARSQKVVEAASHVSAYHNQLLLNLARQLAPTGMVKLFEIDKQFAEMLRDPQNFGLSDQRNACYGGSYVWKPFASRSASTDSQLSAFNPQERLAIAGNPLLAQAVASPMAARSASTLNCEGKMFWDQVHPTTVVHAALSEPAATFIESQYEFLAH

SEQ ID NO: 4 - protein, 336 aa, Aeromonas salmonicida
MKKWFVCLLGLIALTVQAADTRPAFSRIVMFGDSLSDTGKMYSKMRGYLPSSPPYYEGRFSNGPVWLEQLTKQFPGLTIANEAEGGATAVAYNKISWNPKYQVYNNLDYEVTQFLQKDSFKPDDLVILWVGANDYLAYGWNTEQDAKRVRDAISDAANRMVLNGAKQILLFNLPDLGQNPSARSQKVVEAVSHVSAYHNKLLLNLARQLAPTGMVKLFEIDKQFAEMLRDPQNFGLSDVENPCYDGGYVWKPFATRSVSTDRQLSAFSPQERLAIAGNPLLAQAVASPMARRSASPLNCEGKMFWDQVHPTTVVHAALSERAATFIETQYEFLAHG

SEQ ID NO: 5 - protein, 295 aa, Streptomyces coelicolor
MPKPALRRVMTATVAAVGTLALGLTDATAHAAPAQATPTLDYVALGDSYSAGSGVLPVDPANLLCLRSTANYPHVIADTTGARLTDVTCGAAQTADFTRAQYPGVAPQLDALGTGTDLVTLTIGGNDNSTFINAITACGTAGVLSGGKGSPCKDRHGTSFDDEIEANTYPALKEALLGVRARAPHARVAALGYPWITPATADPSCFLKLPLAAGDVPYLRAIQAHLNDAVRRAAEETGATYVDFSGVSDGHDACEAPGTRWIEPLLFGHSLVPVHPNALGERRMAEHTMDVLGLD

SEQ ID NO: 6 - protein, 295 aa, Streptomyces coelicolor; identical to SEQ ID NO: 5

SEQ ID NO: 7 - protein, 238 aa, Saccharomyces cerevisiae
MDYEKFLLFGDSITEFAFNTRPIEDGKDQYALGAALVNEYTRKMDILQRGFKGYTSRWALKILPEILKHESNIVMATIFLGANDACSAGPQSVPLPEFIDNIRQMVSLMKSYHIRPIIIGPGLVDREKWEKEKSEEIALGYFRTNENFAIYSDALAKLANEEKVPFVALNKAFQQEGGDAWQQLLTDGLHFSGKGYKIFHDELLKVIETFYPQYHPKNMQYKLKDWRDVLDDGSNIMS

SEQ ID NO: 8 - protein, 347 aa, Ralstonia sp.
MNLRQWMGAATAALALGLAACGGGGTDQSGNPNVAKVQRMVVFGDSLSDIGTYTPVAQAVGGGKFTTNPGPIWAETVAAQLGVTLTPAVMGYATSVQNCPKAGCFDYAQGGSRVTDPNGIGHNGGAGALTYPVQQQLANFYAASNNTFNGNNDVVFVLAGSNDIFFWTTAAATSGSGVTPAIATAQVQQAATDLVGYVKDMIAKGATQVYVFNLPDSSLTPDGVASGTTGQALLHALVGTFNTTLQSGLAGTSARIIDFNAQLTAAIQNGASFGFANTSARACDATKINALVPSAGGSSLFCSANTLVASGADQSYLFADGVHPTTAGHRLIASNVLARLLADNVAH

SEQ ID NO: 9 - protein, 261 aa, Streptomyces coelicolor
MIGSYVAVGDSFTEGVGDPGPDGAFVGWADRLAVLLADRRPEGDFTYTNLAVRGRLLDQIVAEQVPRVVGLAPDLVSFAAGGNDIIRPGTDPDEVAERFELAVAALTAAAGTVLVTTGFDTRGVPVLKHLRGKIATYNGHVRAIADRYGCPVLDLWSLRSVQDRRAWDADRLHLSPEGHTRVALRAGQALGLRVPADPDQPWPPLPPRGTLDVRRDDVHWAREYLVPWIGRRLRGESSGDHVTAKGTLSPDAIKTRIAAVA

SEQ ID NO: 10 - protein, 260 aa, Streptomyces coelicolor
MQTNPAYTSLVAVGDSFTEGMSDLLPDGSYRGWADLLATRMAARSPGFRYANLAVRGKLIGQIVDEQVDVAAAMGADVITLVGGLNDTLRPKCDMARVRDLLTQAVERLAPHCEQLVLMRSPGRQGPVLERFRPRMEALFAVIDDLAGRHGAVVVDLYGAQSLADPRMWDVDRLHLTAEGHRRVAEAVWQSLGHEPEDPEWHAPIPATPPPGWVTRRTADVRFARQHLLPWIGRRLTGRSSGDGLPAKRPDLLPYEDPAR

SEQ ID NO: 11 - protein, 454 aa, Streptomyces coelicolor
MTRGRDGGAGAPPTKHRALLAAIVTLIVAISAAIYAGASADDGSRDHALQAGGRLPRGDAAPASTGAWVGAWATAPAAAEPGTETTGLAGRSVRNVVHTSVGGTGARITLSNLYGQSPLTVTHASIALAAGPDTAAAIADTMRRLTFGGSARVIIPAGGQVMSDTARLAIPYGANVLVTTYSPIPSGPVTYHPQARQTSYLADGDRTADVTAVAYTTPTPYWRYLTALDVLSHEADGTVVAFGDSITDGARSQSDANHRWTDVLAARLHEAAGDGRDTPRYSVVNEGISGNRLLTSRPGRPADNPSGLSRFQRDVLERTNVKAVVVVLGVNDVLNSPELADRDAILTGLRTLVDRAHARGLRVVGATITPFGGYGGYTEARETMRQEVNEEIRSGRVFDTVVDFDKALRDPYDPRRMRSDYDSGDHLHPGDKGYARMGAVIDLAALKGAAPVKA

SEQ ID NO: 12 - protein, 340 aa, Streptomyces coelicolor
MTSMSRARVARRIAAGAAYGGGGIGLAGAAAVGLVVAEVQLARRRVGVGTPTRVPNAQGLYGGTLPTAGDPPLRLMMLGDSTAAGQGVHRAGQTPGALLASGLAAVAERPVRLGSVAQPGACSDDLDRQVALVLAEPDRVPDICVIMVGANDVTHRMPATRSVRHLSSAVRRLRTAGAEVVVGTCPDLGTIERVRQPLRWLARRASRQLAAAQTIGAVEQGGRTVSLGDLLGPEFAQNPRELFGPDNYHPSAEGYATAAMAVLPSVCAALGLWPADEEHPDALRREGFLPVARAAAEAASEAGTEVAAAMPTGPRGPWALLKRRRRRRVSEAEPSSPSGV

SEQ ID NO: 13 - protein, 305 aa, Streptomyces coelicolor
MGRGTDQRTRYGRRRARVALAALTAAVLGVGVAGCDSVGGDSPAPSGSPSKRTRTAPAWDTSPASVAAVGDSITRGFDACAVLSDCPEVSWATGSSAKVDSLAVRLLGKADAAEHSWNYAVTGARMADLTAQVTRAAQREPELVAVMAGANDACRSTTSAMTPVADFRAQFEEAMATLRKKLPKAQVYVSSIPDLKRLWSQGRTNPLGKQVWKLGLCPSMLGDADSLDSAATLRRNTVRDRVADYNEVLREVCAKDRRCRSDDGAVHEFRFGTDQLSHWDWFHPSVDGQARLAEIAYRAVTAKNP

SEQ ID NO: 14 - protein, 268 aa, Streptomyces rimosus
MRLSRRAATASALLLTPALALFGASAAVSAPRIQATDYVALGDSYSSGVGAGSYDSSSGSCKRSTKSYPALWAASHTGTRFNFTACSGARTGDVLAKQLTPVNSGTDLVSITIGGNDAGFADTMTTCNLQGESACLARIAKARAYIQQTLPAQLDQVYDAIDSRAPAAQVVVLGYPRFYKLGGSCAVGLSEKSRAAINAAADDINAVTAKRAADHGFAFGDVNTTFAGHELCSGAPWLHSVTLPVENSYHPTANGQSKGYLPVLNSAT

SEQ ID NO: 15 - protein, 336 aa, Aeromonas salmonicida
MKKWFVCLLGLIALTVQAADTRPAFSRIVMFGDSLSDTGKMYSKMRGYLPSSPPYYEGRFSNGPVWLEQLTKQFPGLTIANEAEGGATAVAYNKISWNPKYQVINNLDYEVTQFLQKDSFKPDDLVILWVGANDYLAYGWNTEQDAKRVRDAISDAANRMVLNGAKQILLFNLPDLGQNPSARSQKVVEAVSHVSAYHNKLLLNLARQLAPTGMVKLFEIDKQFAEMLRDPQNFGLSDVENPCYDGGYVWKPFATRSVSTDRQLSAFSPQERLAIAGNPLLAQAVASPMARRSASPLNCEGKMFWDQVHPTTVVHAALSERAATFIETQYEFLAHG

SEQ ID NO: 16 - protein, 318 aa, Aeromonas salmonicida
ADTRPAFSRIVMFGDSLSDTGKMYSKMRGYLPSSPPYYEGRFSNGPVWLEQLTKQFPGLTIANEAEGGATAVAYNKISWDPKYQVINNLDYEVTQFLQKDSFKPDDLVILWVGANDYLAYGWNTEQDAKRVRDAISDAANRMVLNGAKQILLFNLPDLGQNPSARSQKVVEAVSHVSAYHNKLLLNLARQLAPTGMVKLFEIDKQFAEMLRDPQNFGLSDVENPCYDGGYVWKPFATRSVSTDRQLSAFSPQERLAIAGNPLLAQAVASPMARRSASPLNCEGKMFWDQVHPTTVVHAALSERAATFIETQYEFLAHG

SEQ ID NO: 17 - protein, 465 aa, Candida parapsilosis
MRYFAIAFLLINTISAFVLAPKKPSQDDFYTPPQGYEAQPLGSILKTRNVPNPLTNVFTPVKVQNAWQLLVRSEDTFGNPNAIVTTIIQPFNAKKDKLVSYQTFEDSGKLDCAPSYAIQYGSDISTLTTQGEMYYISALLDQGYYVVTPDYEGPKSTFTVGLQSGRATLNSLRATLKSGNLTGVSSDAETLLWGYSGGSLASGWAAAIQKEYAPELSKNLLGAALGGFVTNITATAEAVDSGPFAGIISNALAGIGNEYPDFKNYLLKKVSPLLSITYRLGNTHCLLDGGIAYFGKSFFSRIIRYFPDGWDLVNQEPIKTILQDNGLVYQPKDLTPQIPLFIYHGTLDAIVPIVNSRKTFQQWCDWGLKSGEYNEDLTNGHITESIVGAPAALTWIINRFNGQPPVDGCQHNVRASNLEYPGTPQSIKNYFEAALHAILGFDLGPDVKRDKVTLGGLLKLERFAF

SEQ ID NO: 18 - protein, 471 aa, Candida parapsilosis
MRYFAIAFLLINTISAFVLAPKKPSQDDFYTPPQGYEAQPLGSILKTRNVPNPLTNVFTPVKVQNAWQLLVRSEDTFGNPNAIVTTIIQPFNAKKDKLVSYQTFEDSGKLDCAPSYAIQYGSDISTLTTQGEMYYISALLDQGYYVVTPDYEGPKSTFTVGLQSGRATLNSLRATLKSGNLTGVSSDAETLLWGYSGGSLASGWAAAIQKEYAPELSKNLLGAALGGFVTNITATAEAVDSGPFAGIISNALAGIGNEYPDFKNYLLKKVSPLLSITYRLGNTHCLLDGGIAYFGKSFFSRIIRYFPDGWDLVNQEPIKTILQDNGLVYQPKDLTPQIPLFIYHGTLDAIVPIVNSRKTFQQWCDWGLKSGEYNEDLTNGHITESIVGAPAALTWIINRFNGQPPVDGCQHNVRASNLEYPGTPQSIKNYFEAALHAILGFDLGPDVKRDKVTLGGLLKLERFAFHHHHHH

SEQ ID NO: 19 - protein, 261 aa, Streptomyces coelicolor; identical to SEQ ID NO: 9

SEQ ID NO: 20 - protein, 4 aa, Artificial Sequence
  Description of Artificial Sequence Synthetic peptide
GDSX

SEQ ID NO: 21 - protein, 5 aa, Artificial Sequence
  Description of Artificial Sequence Synthetic peptide
GXNDX

SEQ ID NO: 22 - protein, 4 aa, Artificial Sequence
  Description of Artificial Sequence Synthetic peptide
DXXH

SEQ ID NO: 23 - protein, 5 aa, Artificial Sequence
  Description of Artificial Sequence Synthetic peptide
RRSAS

SEQ ID NO: 24 - DNA, 1047 bp, Aeromonas hydrophila
atgtttaagtttaaaaagaatttcttagttggattatcggcagctttaatgagtattagcttgttttcggcaaccgcctctgcagctagcgccgacagccgtcccgccttttcccggatcgtgatgttcggcgacagcctctccgataccggcaaaatgtacagcaagatgcgcggttacctcccctccagcccgccctactatgagggccgtttctccaacggacccgtctggctggagcagctgaccaaacagttcccgggtctgaccatcgccaacgaagcggaaggcggtgccactgccgtggcttacaacaagatctcctggaatcccaagtatcaggtcatcaacaacctggactacgaggtcacccagttcttgcagaaagacagcttcaagccggacgatctggtgatcctctgggtcggtgccaatgactatctggcctatggctggaacacggagcaggatgccaagcgggttcgcgatgccatcagcgatgcggccaaccgcatggtactgaacggtgccaagcagatactgctgttcaacctgccggatctgggccagaacccgtcagctcgcagtcagaaggtggtcgaggcggtcagccatgtctccgcctatcacaaccagctgctgctgaacctggcacgccagctggcccccaccggcatggtaaagctgttcgagatcgacaagcaatttgccgagatgctgcgtgatccgcagaacttcggcctgagcgacgtcgagaacccctgctacgacggcggctatgtgtggaagccgtttgccacccgcagcgtcagcaccgaccgccagctctccgccttcagtccgcaggaacgcctcgccatcgccggcaacccgctgctggcacaggccgttgccagtcctatggcccgccgcagcgccagccccctcaactgtgagggcaagatgttctgggatcaggtacacccgaccactgtcgtgcacgcagccctgagcgagcgcgccgccaccttcatcgcgaaccagtacgagttcctcgcccactgatga

SEQ ID NO: 25 - protein, 347 aa, Artificial Sequence
  Description of Artificial Sequence Synthetic fusion construct polypeptide used for mutagenesis
MFKFKKNFLVGLSAALMSISLFSATASAASADSRPAFSRIVMFGDSLSDTGKMYSKMRGYLPSSPPYYEGRFSNGPVWLEQLTKQFPGLTIANEAEGGATAVAYNKISWNPKYQVINNLDYEVTQFLQKDSFKPDDLVILWVGANDYLAYGWNTEQDAKRVRDAISDAANRMVLNGAKQILLFNLPDLGQNPSARSQKVVEAVSHVSAYHNQLLLNLARQLAPTGMVKLFEIDKQFAEMLRDPQNFGLSDVENPCYDGGYVWKPFATRSVSTDRQLSAFSPQERLAIAGNPLLAQAVASPMARRSASPLNCEGKMFWDQVHPTTVVHAALSERAATFIANQYEFLAH

SEQ ID NO: 26 - protein, 267 aa, Streptomyces sp.
MRLTRSLSAASVIVFALLLALLGISPAQAAGPAYVALGDSYSSGNGAGSYIDSSGDCHRSNNAYPARWAAANAPSSFTFAACSGAVTTDVINNQLGALNASTGLVSITIGGNDAGFADAMTTCVTSSDSTCLNRLATATNYINTTLLARLDAVYSQIKARAPNARVVVLGYPRMYLASNPWYCLGLSNTKRAAINTTADTLNSVISSRATAHGFRFGDVRPTFNNHELFFGNDWLHSLTLPVWESYHPTSTGHQSGYLPVLNANSST

SEQ ID NO: 27 - protein, 548 aa, Thermobifida sp.
MLPHPAGERGEVGAFFALLVGTPQDRRLRLECHETRPLRGRCGCGERRVPPLTLPGDGVLCTTSSTRDAETVWRKHLQPRPDGGFRPHLGVGCLLAGQGSPGVLWCGREGCRFEVCRRDTPGLSRTRNGDSSPPFRAGWSLPPKCGEISQSARKTPAVPRYSLLRTDRPDGPRGRFVGSGPRAATRRRLFLGIPALVLVTALTLVLAVPTGRETLWRMWCEATQDWCLGVPVDSRGQPAEDGEFLLLSPVQAATWGNYYALGDSYSSGDGARDYYPGTAVKGGCWRSANAYPELVAEAYDFAGHLSFLACSGQRGYAMLDAIDEVGSQLDWNSPHTSLVTIGIGGNDLGFSTVLKTCMVRVPLLDSKACTDQEDAIRKRMAKFETTFEELISEVRTRAPDARILVVGYPRIFPEEPTGAYYTLTASNQRWLNETIQEFNQQLAEAVAVHDEEIAASGGVGSVEFVDVYHALDGHEIGSDEPWVNGVQLRDLATGVTVDRSTFHPNAAGHRAVGERVIEQIETGPGRPLYATFAVVAGATVDTLAGEVG

SEQ ID NO: 28 - protein, 372 aa, Thermobifida sp.
MGSGPRAATRRRLFLGIPALVLVTALTLVLAVPTGRETLWRMWCEATQDWCLGVPVDSRGQPAEDGEFLLLSPVQAATWGNYYALGDSYSSGDGARDYYPGTAVKGGCWRSANAYPELVAEAYDFAGHLSFLACSGQRGYAMLDAIDEVGSQLDWNSPHTSLVTIGIGGNDLGFSTVLKTCMVRVPLLDSKACTDQEDAIRKRMAKFETTFEELISEVRTRAPDARILVVGYPRIFPEEPTGAYYTLTASNQRWLNETIQEFNQQLAEAVAVHDEEIAASGGVGSVEFVDVYHALDGHEIGSDEPWVNGVQLRDLATGVTVDRSTFHPNAAGHRAVGERVIEQIETGPGRPLYATFAVVAGATVDTLAGEVG

SEQ ID NO: 29 - protein, 300 aa, Corynebacterium efficiens
MRTTVIAASALLLLAGCADGAREETAGAPPGESSGGIREEGAEASTSITDVYIALGDSYAAMGGRDQPLRGEPFCLRSSGNYPELLHAEVTDLTCQGAVTGDLLEPRTLGERTLPAQVDALTEDTTLVTLSIGGNDLGFGEVAGCIRERIAGENADDCVDLLGETIGEQLDQLPPQLDRVHEAIRDRAGDAQVVVTGYLPLVSAGDCPELGDVSEADRRWAVELTGQINETVREAAERHDALFVLPDDADEHTSCAPPQQRWADIQGQQTDAYPLHPTSAGHEAMAAAVRDALGLEPVQP

SEQ ID NO: 30 - protein, 284 aa, Novosphingobium aromaticivorans
MGQVKLFARRCAPVLLALAGLAPAATVAREAPLAEGARYVALGSSFAAGPGVGPNAPGSPERCGRGTLNYPHLLAEALKLDLVDATCSGATTHHVLGPWNEVPPQIDSVNGDTRLVTLTIGGNDVSFVGNIFAAACEKMASPDPRCGKWREITEEEWQADEERMRSIVRQIHARAPLARVVVVDYITVLPPSGTCAAMAISPDRLAQSRSAAKRLARITARVAREEGASLLKFSHISRRHHPCSAKPWSNGLSAPADDGIPVHPNRLGHAEAAAALVKLVKLMK

SEQ ID NO: 31 - protein, 268 aa, Streptomyces coelicolor
MRRFRLVGFLSSLVLAAGAALTGAATAQAAQPAAADGYVALGDSYSSGVGAGSYISSSGDCKRSTKAHPYLWAAAHSPSTFDFTACSGARTGDVLSGQLGPLSSGTGLVSISIGGNDAGFADTMTTCVLQSESSCLSRIATAEAYVDSTLPGKLDGVYSAISDKAPNAHVVVIGYPRFYKLGTTCIGLSETKRTAINKASDHLNTVLAQRAAAHGFTFGDVRTTFTGHELCSGSPWLHSVNWLNIGESYHPTAAGQSGGYLPVLNGAA

SEQ ID NO: 32 - protein, 269 aa, Streptomyces avermitilis
MRRSRITAYVTSLLLAVGCALTGAATAQASPAAAATGYVALGDSYSSGVGAGSYLSSSGDCKRSSKAYPYLWQAAHSPSSFSFMACSGARTGDVLANQLGTLNSSTGLVSLTIGGNDAGFSDVMTTCVLQSDSACLSRINTAKAYVDSTLPGQLDSVYTAISTKAPSAHVAVLGYPRFYKLGGSCLAGLSETKRSAINDAADYLNSAIAKRAADHGFTFGDVKSTFTGHEICSSSTWLHSLDLLNIGQSYHPTAAGQSGGYLPVMNSVA

SEQ ID NO: 33 - protein, 267 aa, Streptomyces sp.; identical to SEQ ID NO: 26

SEQ ID NO: 34 - protein, 317 aa, Aeromonas hydrophila
ADSRPAFSRIVMFGDSLSDTGKMYSKMRGYLPSSPPYYEGRFSNGPVWLEQLTNEFPGLTIANEAEGGPTAVAYNKISWNPKYQVINNLDYEVTQFLQKDSFKPDDLVILWVGANDYLAYGWNTEQDAKRVRDAISDAANRMVLNGAKEILLFNLPDLGQNPSARSQKVVEAASHVSAYHNQLLLNLARQLAPTGMVKLFEIDKQFAEMLRDPQNFGLSDQRNACYGGSYVWKPFASRSASTDSQLSAFNPQERLAIAGNPLLAQAVASPMAARSASTLNCEGKMFWDQVHPTTVVHAALSEPAATFIESQYEFLAH

SEQ ID NO: 35 - protein, 318 aa, Aeromonas salmonicida
ADTRPAFSRIVMFGDSLSDTGKMYSKMRGYLPSSPPYYEGRFSNGPVWLEQLTKQFPGLTIANEAEGGATAVAYNKISWNPKYQVINNLDYEVTQFLQKDSFKPDDLVILWVGANDYLAYGWNTEQDAKRVRDAISDAANRMVLNGAKQILLFNLPDLGQNPSARSQKVVEAVSHVSAYHNKLLLNLARQLAPTGMVKLFEIDKQFAEMLRDPQNFGLSDVENPCYDGGYVWKPFATRSVSTDRQLSAFSPQERLAIAGNPLLAQAVASPMARRSASPLNCEGKMFWDQVHPTTVVHAALSERAATFIETQYEFLAHG

SEQ ID NO: 36 - DNA, 1371 bp, Streptomyces thermosacchari
acaggccgatgcacggaaccgtacctttccgcagtgaagcgctctccccccatcgttcgccgggacttcatccgcgattttggcatgaacacttccttcaacgcgcgtagcttgctacaagtgcggcagcagacccgctcgttggaggctcagtgagattgacccgatccctgtcggccgcatccgtcatcgtcttcgccctgctgctcgcgctgctgggcatcagcccggcccaggcagccggcccggcctatgtggccctgggggattcctattcctcgggcaacggcgccggaagttacatcgattcgagcggtgactgtcaccgcagcaacaacgcgtaccccgcccgctgggcggcggccaacgcaccgtcctccttcaccttcgcggcctgctcgggagcggtgaccacggatgtgatcaacaatcagctgggcgccctcaacgcgtccaccggcctggtgagcatcaccatcggcggcaatgacgcgggcttcgcggacgcgatgaccacctgcgtcaccagctcggacagcacctgcctcaaccggctggccaccgccaccaactacatcaacaccaccctgctcgcccggctcgacgcggtctacagccagatcaaggcccgtgcccccaacgcccgcgtggtcgtcctcggctacccgcgcatgtacctggcctcgaacccctggtactgcctgggcctgagcaacaccaagcgcgcggccatcaacaccaccgccgacaccctcaactcggtgatctcctcccgggccaccgcccacggattccgattcggcgatgtccgcccgaccttcaacaaccacgaactgttcttcggcaacgactggctgcactcactcaccctgccggtgtgggagtcgtaccaccccaccagcacgggccatcagagcggctatctgccggtcctcaacgccaacagctcgacctgatcaacgcacggccgtgcccgccccgcgcgtcacgctcggcgcgggcgccgcagcgcgttgatcagcccacagtgccggtgacggtcccaccgtcacggtcgagggtgtacgtcacggtggcgccgctccagaagtggaacgtcagcaggaccgtggagccgtccctgacctcgtcgaagaactccggggtcagcgtgatcacccctcccccgtagccgggggcgaaggcggcgccgaactccttgtaggacgtccagtcgtgcggcccggcgttgccaccgtccgcgtagaccgcttccatggtcgccagccggtccccgcggaactcggtggggatgtccgtgcccaaggtggtcccggtggtgtccgagagcaccgggggctcgtaccggatgatgtgcagatccaaagaatt

SEQ ID NO: 37 - protein, 267 aa, Streptomyces thermosacchari; identical to SEQ ID NO: 26

SEQ ID NO: 38 - protein, 548 aa, Thermobifida fusca; identical to SEQ ID NO: 27

SEQ ID NO: 39 - DNA, 3000 bp, Thermobifida fusca
ggtggtgaaccagaacacccggtcgtcggcgtgggcgtccaggtgcaggtgcaggttcttcaactgctccagcaggatgccgccgtggccgtgcacgatggccttgggcaggcctgtggtccccgacgagtacagcacccatagcggatggtcgaacggcagcggggtgaactccagttccgcgccttcgcccgcggcttcgaactccgcccaggacagggtgtcggcgacagggccgcagcccaggtacggcaggacgacggtgtgctgcaggctgggcatgccgtcgcgcagggctttgagcacgtcacggcggtcgaagtccttaccgccgtagcggtagccgtccacggccagcagcactttcggttcgatctgcgcgaaccggtcgaggacgctgcgcaccccgaagtcgggggaacaggacgaccaggtcgcaccgatcgcggcgcaggcgaggaatgcggccgtcgcctcggcgatgttcggcaggtaggccacgacccggtcgccggggcccaccccgaggctgcggagggccgcagcgatcgcggcggtgcgggtccgcagttctccccaggtccactcggtcaacggccggagttcggacgcgtgccggatcgccacggctgatgggtcacggtcgcggaagatgtgctcggcgtagttgagggtggcgccggggaaccagacggcgccgggcatggcgtcggaggcgagcactgtggtgtacggggtggcggcgcgcacccggtagtactcccagatcgcggaccagaatccttcgaggtcggttaccgaccagcgccacagtgcctcgtagtccggtgcgtccacaccgcggtgctcccgcacccagcgggtgaacgcggtgaggttggcgcgttctttgcgctcctcgtcgggactccacaggatcggcggctgcggcttgagtgtcatgaaacgcgaccccttcgtggacggtgcggatgcggtgagcgtcgggtgcctcccctaacgctccccggtgacggagtgttgtgcaccacatctagcacgcgggacgcggaaaccgtatggagaaaacacctacaaccccggccggacggtgggtttcggccacacttaggggtcgggtgcctgcttgccgggcagggcagtcccggggtgctgtggtgcgggcgggagggctgtcgcttcgaggtgtgccggcgggacactccgggcctcagccgtacccgcaacggggacagttctcctcccttccgggctggatggtcccttcccccgaaatgcggcgagatctcccagtcagcccggaaaacacccgctgtgcccaggtactctttgcttcgaacagacaggccggacggtccacgggggaggtttgtgggcagcggaccacgtgcggcgaccagacgacggttgttcctcggtatccccgctcttgtacttgtgacagcgctcacgctggtcttggctgtcccgacggggcgcgagacgctgtggcgcatgtggtgtgaggccacccaggactggtgcctgggggtgccggtcgactcccgcggacagcctgcggaggacggcgagtttctgctgctttctccggtccaggcagcgacctgggggaactattacgcgctcggggattcgtactcttcgggggacggggcccgcgactactatcccggcaccgcggtgaagggcggttgctggcggtccgctaacgcctatccggagctggtcgccgaagcctacgacttcgccggacacttgtcgttcctggcctgcagcggccagcgcggctacgccatgcttgacgctatcgacgaggtcggctcgcagctggactggaactcccctcacacgtcgctggtgacgatcgggatcggcggcaacgatctggggttctccacggttttgaagacctgcatggtgcgggtgccgctgctggacagcaaggcgtgcacggaccaggaggacgctatccgcaagcggatggcgaaattcgagacgacgtttgaagagctcatcagcgaagtgcgcacccgcgcgccggacgcccggatccttgtcgtgggctacccccggatttttccggaggaaccgaccggcgcctactacacgctgaccgcgagcaaccagcggtggctcaacgaaaccattcaggagttcaaccagcagctcgccgaggctgtcgcggtccacgacgaggagattgccgcgtcgggcggggtgggcagcgtggagttcgtggacgtctaccacgcgttggacggccacgagatcggctcggacgagccgtgggtgaacggggtgcagttgcgggacctcgccaccggggtgactgtggaccgcagtaccttccaccccaacgccgctgggcaccgggcggtcggtgagcgggtcatcgagcagatcgaaaccggcccgggccgtccgctctatgccactttcgcggtggtggcgggggcgaccgtggacactctcgcgggcgaggtggggtgacccggcttaccgtccggcccgcaggtctgcgagcactgcggcgatctggtccactgcccagtgcagttcgtcttcggtgatgaccagcggcggggagagccggatcgttgagccgtgcgtgtctttgacgagcacaccccgctgcaggagccgttcgcacagttctcttccggtggccagagtcgggtcgacgtcgatcccagcccacaggccgatgctgcgggccgcgaccacgccgttgccgaccagttggtcgaggcgggcgcgcagcacgggggcgagggcgcggacatggtccaggtaagggccgtcgcggacgaggctcaccacggcagtgccgaccgcgcaggcgagggcgttgccgccgaaggtgctgccgtgctggccggggcggatcacgtcgaagacttccgcgtcgcctaccgccgccgccacgggcaggatgccgccgcccagcgctttgccgaacaggtagatatcggcgtcgactccgctgtggtcgcaggcccg

SEQ ID NO: 40 - protein, 372 aa, Thermobifida fusca
VGSGPRAATRRRLFLGIPALVLVTALTLVLAVPTGRETLWRMWCEATQDWCLGVPVDSRGQPAEDGEFLLLSPVQAATWGNYYALGDSYSSGDGARDYYPGTAVKGGCWRSANAYPELVAEAYDFAGHLSFLACSGQRGYAMLDAIDEVGSQLDWNSPHTSLVTIGIGGNDLGFSTVLKTCMVRVPLLDSKACTDQEDAIRKRMAKFETTFEELISEVRTRAPDARILVVGYPRIFPEEPTGAYYTLTASNQRWLNETIQEFNQQLAEAVAVHDEEIAASGGVGSVEFVDVYHALDGHEIGSDEPWVNGVQLRDLATGVTVDRSTFHPNAAGHRAVGERVIEQIETGPGRPLYATFAVVAGATVDTLAGEVG

SEQ ID NO: 41 - protein, 300 aa, Corynebacterium efficiens; identical to SEQ ID NO: 29

SEQ ID NO: 42 - DNA, 3000 bp, Corynebacterium efficiens
ttctggggtgttatggggttgttatcggctcgtcctgggtggatcccgccaggtggggtattcacgggggacttttgtgtccaacagccgagaatgagtgccctgagcggtgggaatgaggtgggcggggctgtgtcgccatgagggggcggcgggctctgtggtgccccgcgacccccggccccggtgagcggtgaatgaaatccggctgtaatcagcatcccgtgcccaccccgtcggggaggtcagcgcccggagtgtctacgcagtcggatcctctcggactcggccatgctgtcggcagcatcgcgctcccgggtcttggcgtccctcggctgttctgcctgctgtccctggaaggcgaaatgatcaccggggagtgatacaccggtggtctcatcccggatgcccacttcggcgccatccggcaattcgggcagctccgggtggaagtaggtggcatccgatgcgtcggtgacgccatagtgggcgaagatctcatcctgctcgagggtgctcaggccactctccggatcgatatcgggggcgtccttgatggcgtccttgctgaaaccgaggtgcagcttgtgggcttccaatttcgcaccacggagcgggacgaggctggaatgacggccgaagagcccgtggtggacctcaacgaaggtgggtagtcccgtgtcatcattgaggaacacgccctccaccgcacccagcttgtggccggagttgtcgtaggcgctggcatccagaagggaaacgatctcatatttgtcggtgtgctcagacatgatcttcctttgctgtcggtgtctggtactaccacggtagggctgaatgcaactgttatttttctgttattttaggaattggtccatatcccacaggctggctgtggtcaaatcgtcatcaagtaatccctgtcacacaaaatgggtggtgggagccctggtcgcggttccgtgggaggcgccgtgccccgcaggatcgtcggcatcggcggatctggccggtaccccgcggtgaataaaatcattctgtaaccttcatcacggttggttttaggtatccgcccctttcgtcctgaccccgtccccggcgcgcgggagcccgcgggttgcggtagacaggggagacgtggacaccatgaggacaacggtcatcgcagcaagcgcattactccttctcgccggatgcgcggatggggcccgggaggagaccgccggtgcaccgccgggtgagtcctccgggggcatccgggaggagggggcggaggcgtcgacaagcatcaccgacgtctacatcgccctcggggattcctatgcggcgatgggcgggcgggatcagccgttacggggtgagccgttctgcctgcgctcgtccggtaattacccggaactcctccacgcagaggtcaccgatctcacctgccagggggcggtgaccggggatctgctcgaacccaggacgctgggggagcgcacgctgccggcgcaggtggatgcgctgacggaggacaccaccctggtcaccctctccatcgggggcaatgacctcggattcggggaggtggcgggatgcatccgggaacggatcgccggggagaacgctgatgattgcgtggacctgctgggggaaaccatcggggagcagctcgatcagcttcccccgcagctggaccgcgtgcacgaggctatccgggaccgcgccggggacgcgcaggttgtggtcaccggttacctgccgctcgtgtctgccggggactgccccgaactgggggatgtctccgaggcggatcgtcgttgggcggttgagctgaccgggcagatcaacgagaccgtgcgcgaggcggccgaacgacacgatgccctctttgtcctgcccgacgatgccgatgagcacaccagttgtgcacccccacagcagcgctgggcggatatccagggccaacagaccgatgcctatccgctgcacccgacctccgccggccatgaggcgatggccgccgccgtccgggacgcgctgggcctggaaccggtccagccgtagcgccgggcgcgcgcttgtcgacgaccaacccatgccaggctgcagtcacatccgcacatagcgcgcgcgggcgatggagtacgcaccatagaggatgagcccgatgccgacgatgatgagcagcacactgccgaagggttgttccccgagggtgcgcagagccgagtccagacctgcggcctgctccggatcatgggcccaaccggcgatgacgatcaacacccccaggatcccgaaggcgataccacgggcgacataaccggctgttccggtgatgatgatcgcggtcccgacctgccctgaccccgcacccgcctccagatcctcccggaaatcccgggtggcccccttccagaggttgtagacacccgcccccagtaccaccagcccggcgaccacaaccagcaccacaccccagggttgggataggacggtggcggtgacatcggtggcggtctccccatcggaggtgctgccgccccgggcgaaggtggaggtggtcaccgccagggagaagtagaccatggccatgaccgcccccttggccctttccttgaggtcctcgcccgccagcagctggctcaattgccagagtcccagggccgccagggcgatgacggcaacccacaggaggaactgcccacccggagcctccgcgatggtggccagggcacctgaattcgaggcctcatcacccgaaccgccggatccagtggcgatgcgcaccgcgatccacccgatgaggatgtgcagtatgcccaggacaatgaaaccacctctggccagggtggtcagcgcggggtggtcctcggcctggtcggcagcccgttcgatcgtccgtttcgcggatctggtgtcgcccttatccatagctcccattgaaccgccttgaggggtgggcggccactgtcagggcggattgtgatctgaactgtgatgttccatcaaccc

SEQ ID NO: 43 - protein, 268 aa, Streptomyces coelicolor; identical to SEQ ID NO: 31

SEQ ID NO: 44 - DNA, 2000 bp, Streptomyces coelicolor
cccggcggcccgtgcaggagcagcagccggcccgcgatgtcctcgggcgtcgtcttcatcaggccgtccatcgcgtcggcgaccggcgccgtgtagttggcccggacctcgtcccaggtgcccgcggcgatctggcgggtggtgcggtgcgggccgcgccgaggggagacgtaccagaagcccatcgtcacgttctccggctgcggttcgggctcgtccgccgctccgtccgtcgcctcgccgagcaccttctcggcgaggtcggcgctggtcgccgtcaccgtgacgtcggcgccccggctccagcgcgagatcagcagcgtccagccgtcgccctccgccagcgtcgcgctgcggtcgtcgtcgcgggcgatccgcagcacgcgcgcgccgggcggcagcagcgtggcgccggaccgtacgcggtcgatgttcgccgcgtgcgagtacggctgctcacccgtggcgaaacggccgaggaacagcgcgtcgacgacgtcggacggggagtcgctgtcgtccacgttgagccggatcggcagggcttcgtgcgggttcacggacatgtcgccatgatcgggcacccggccgccgcgtgcacccgctttcccgggcacgcacgacaggggctttctcgccgtcttccgtccgaacttgaacgagtgtcagccatttcttggcatggacacttccagtcaacgcgcgtagctgctaccacggttgtggcagcaatcctgctaagggaggttccatgagacgtttccgacttgtcggcttcctgagttcgctcgtcctcgccgccggcgccgccctcaccggggcagcgaccgcccaggcggcccaacccgccgccgccgacggctatgtggccctcggcgactcctactcctccggggtcggagcgggcagctacatcagctcgagcggcgactgcaagcgcagcacgaaggcccatccctacctgtgggcggccgcccactcgccctccacgttcgacttcaccgcctgttccggcgcccgtacgggtgatgttctctccggacagctcggcccgctcagctccggcaccggcctcgtctcgatcagcatcggcggcaacgacgccggtttcgccgacaccatgacgacctgtgtgctccagtccgagagctcctgcctgtcgcggatcgccaccgccgaggcgtacgtcgactcgacgctgcccggcaagctcgacggcgtctactcggcaatcagcgacaaggcgccgaacgcccacgtcgtcgtcatcggctacccgcgcttctacaagctcggcaccacctgcatcggcctgtccgagaccaagcggacggcgatcaacaaggcctccgaccacctcaacaccgtcctcgcccagcgcgccgccgcccacggcttcaccttcggcgacgtacgcaccaccttcaccggccacgagctgtgctccggcagcccctggctgcacagcgtcaactggctgaacatcggcgagtcgtaccaccccaccgcggccggccagtccggtggctacctgccggtcctcaacggcgccgcctgacctcaggcggaaggagaagaagaaggagcggagggagacgaggagtgggaggccccgcccgacggggtccccgtccccgtctccgtctccgtcccggtcccgcaagtcaccgagaacgccaccgcgtcggacgtggcccgcaccggactccgcacctccacgcgcacggcactctcgaacgcgccggtgtcgtcgtgcgtcgtcaccaccacgccgtcctggcgcgagcgctcgccgcccgacgggaaggacagcgtccgccaccccggatcggagaccgacccgtccgcggtcacccaccggtagccgacctccgcgggcagccgcccgaccgtgaacgtcgccgtgaacgcgggtgcccggtcgtgcggcggcggacaggcccccgagtagtgggtgcgcgagcccaccacggtcacctccaccgactgcgctgcggggc

SEQ ID NO: 45 - protein, 269 aa, Streptomyces avermitilis; identical to SEQ ID NO: 32

SEQ ID NO: 46 - DNA, 1980 bp, Streptomyces avermitilis
ccaccgccgggtcggcggcgagtctcctggcctcggtcgcggagaggttggccgtgtagccgttcagcgcggcgccgaacgtcttcttcaccgtgccgccgtactcgttgatcaggcccttgcccttgctcgacgcggccttgaagccggtgcccttcttgagcgtgacgatgtagctgcccttgatcgcggtgggggagccggcggcgagcaccgtgccctcggccggggtggcctgggcgggcagtgcggtgaatccgcccacgagggcgccggtcgccacggcggttatcgcggcgatccggatcttcttgctacgcagctgtgccatacgagggagtcctcctctgggcagcggcgcgcctgggtggggcgcacggctgtggggggtgcgcgcgtcatcacgcacacggccctggagcgtcgtgttccgccctgggttgagtaaagcctcggccatctacgggggtggctcaagggagttgagaccctgtcatgagtctgacatgagcacgcaatcaacggggccgtgagcaccccggggcgaccccggaaagtgccgagaagtcttggcatggacacttcctgtcaacacgcgtagctggtacgacggttacggcagagatcctgctaaagggaggttccatgagacgttcccgaattacggcatacgtgacctcactcctcctcgccgtcggctgcgccctcaccggggcagcgacggcgcaggcgtccccagccgccgcggccacgggctatgtggccctcggcgactcgtactcgtccggtgtcggcgccggcagctacctcagctccagcggcgactgcaagcgcagttcgaaggcctatccgtacctctggcaggccgcgcattcaccctcgtcgttcagtttcatggcttgctcgggcgctcgtacgggtgatgtcctggccaatcagctcggcaccctgaactcgtccaccggcctggtctccctcaccatcggaggcaacgacgcgggcttctccgacgtcatgacgacctgtgtgctccagtccgacagcgcctgcctctcccgcatcaacacggcgaaggcgtacgtcgactccaccctgcccggccaactcgacagcgtgtacacggcgatcagcacgaaggccccgtcggcccatgtggccgtgctgggctacccccgcttctacaaactgggcggctcctgcctcgcgggcctctcggagaccaagcggtccgccatcaacgacgcggccgactatctgaacagcgccatcgccaagcgcgccgccgaccacggcttcaccttcggcgacgtcaagagcaccttcaccggccatgagatctgctccagcagcacctggctgcacagtctcgacctgctgaacatcggccagtcctaccacccgaccgcggccggccagtccggcggctatctgccggtcatgaacagcgtggcctgagctcccacggcctgaatttttaaggcctgaatttttaaggcgaaggtgaaccggaagcggaggccccgtccgtcggggtctccgtcgcacaggtcaccgagaacggcacggagttggacgtcgtgcgcaccgggtcgcgcacctcgacggcgatctcgttcgagatcgttccgctcgtgtcgtacgtggtgacgaacacctgcttctgctgggtctttccgccgctcgccgggaaggacagcgtcttccagcccggatccgggacctcgcccttcttggtcacccagcggtactccacctcgaccggcacccggcccaccgtgaaggtcgccgtgaacgtgggcgcctgggcggtgggcggcgggcaggcaccggagtagtcggtgtgcacgccggtgaccgtcaccttcacggactgggccggcggggtcgtcgtaccgccgccgccaccgccgcctcccggagtggagcccgagctgtggtcgcccccgccgtcggcgttgtcgtcctcgggggttttcgaac

SEQ ID NO: 47 - protein, 372 aa, Thermobifida fusca; identical to SEQ ID NO: 28

SEQ ID NO: 48 - DNA, 968 bp, Thermobifida fusca
ctgcagacacccgccccgccttctcccggatcgtcatgttcggcgactccctcagcgacaccggcaagatgtactccaagatgcgcggctacctgccgtcctccccgccgtactacgagggccgcttctcgaacggcccggtctggctggagcagctgacgaagcagttccccggcctgacgatcgccaacgaggccgaggggggcgcgaccgcagtcgcctacaacaagatctcctggaacccgaagtaccaggtcattaacaacctcgactacgaggtcacccagttcttgcagaaggactcgttcaagcccgacgacctggtcatcctgtgggtgggcgccaacgactacctggcctacggttggaacacggagcaggacgccaagcgggtgcgcgacgccatctcggacgcggcaaaccgcatggtcctgaacggcgcgaagcagatcctgctgttcaacctgcccgacctgggccagaacccgtccgcccgctcccagaaggtcgtcgaggccgtctcgcacgtgtccgcctaccacaacaagctgctcctcaacctcgcccggcagctcgccccgacgggcatggtcaagctgttcgagatcgacaagcagttcgcggagatgctgcgcgacccccagaacttcggcctgagcgacgtggagaacccgtgctacgacggcggctacgtgtggaagccgttcgccacccggtccgtctcgaccgaccggcagctgtcggccttctcgccccaggagcgcctggcgatcgctggcaacccgctcctggcacaggcggtagcttcgccgatggcccgccgctcggcctcgcccctcaactgcgagggcaagatgttctgggaccaggtccaccccaccaccgtggtccacgccgccctctcggagcgcgccgccaccttcatcgagacccagtacgagttcctcgcccactagtctagaggatcc

SEQ ID NO: 49 - DNA, 1044 bp, Aeromonas salmonicida
atgaaacaacaaaaacggctttacgcccgattgctgacgctgttatttgcgctcatcttcttgctgcctcattctgcagcttcagcagcagatacaagaccggcgtttagccggatcgtcatgtttggagatagcctgagcgatacgggcaaaatgtatagcaaaatgagaggctatcttccgtcaagcccgccgtattatgaaggccgctttagcaatggaccggtctggctggaacaactgacgaaacaatttccgggactgacgatcgctaatgaagcagaaggaggagcaacagcggtcgcctataacaaaatcagctgggacccgaaatatcaggtcatcaacaacctggactatgaagtcacacagtttcttcagaaagacagctttaaaccggatgatctggtcatcctttgggtcggcgccaatgattatctggcgtatggctggaacacagaacaagatgccaaaagagtcagagatgccatcagcgatgccgctaatagaatggtcctgaacggcgccaaacaaatcctgctgtttaacctgccggatctgggacaaaatccgagcgccagaagccaaaaagtcgtcgaagcagtcagccatgtcagcgcctatcataacaaactgctgctgaacctggcaagacaattggcaccgacgggaatggttaaattgtttgaaattgacaaacagtttgccgaaatgctgagagatccgcaaaattttggcctgagcgatgtcgaaaacccgtgctatgatggcggatatgtctggaaaccgtttgccacaagaagcgtcagcacggatagacaactgtcagcgtttagcccgcaagaaagactggcaatcgccggaaatccgcttttggcacaagcagttgcttcaccgatggcaagaagatcagcaagcccgctgaattgcgaaggcaaaatgttttgggatcaggtccatccgacaacagttgtccatgctgccctttcagaaagagcggcgacgtttatcgaaacacagtatgaatttctggcccatggctga

SEQ ID NO: 50 - DNA, 1005 bp, Aeromonas hydrophila
atgaaaaaatggtttgtgtgtttattgggattggtcgcgctgacagttcaggcagccgacagccgtcccgccttctcccggatcgtgatgtttggcgacagcctctccgataccggcaagatgtacagcaagatgcgcggttacctcccctccagccccccctactatgagggccgcttctccaacgggcccgtctggctggagcagctgaccaacgagttcccgggcctgaccatagccaacgaggcggaaggcggaccgaccgccgtggcttacaacaagatctcctggaatcccaagtatcaggtcatcaacaacctggactacgaggtcacccagttcctgcaaaaagacagcttcaagccggacgatctggtgatcctctgggtcggcgccaacgactatctggcctatggctggaacacagagcaggatgccaagcgggtgcgcgacgccatcagcgatgcggccaaccgcatggtgctgaacggcgccaaggagatactgctgttcaacctgccggatctgggccagaacccctcggcccgcagccagaaggtggtcgaggcggccagccatgtctccgcctaccacaaccagctgctgctgaacctggcacgccagctggctcccaccggcatggtgaagctgttcgagatcgacaagcagtttgccgagatgctgcgtgatccgcagaacttcggcctgagcgaccagaggaacgcctgctacggtggcagctatgtatggaagccgtttgcctcccgcagcgccagcaccgacagccagctctccgccttcaacccgcaggagcgcctcgccatcgccggcaacccgctgctggcccaggccgtcgccagccccatggctgcccgcagcgccagcaccctcaactgtgagggcaagatgttctgggatcaggtccaccccaccactgtcgtgcacgccgccctgagcgagcccgccgccaccttcatcgagagccagtacgagttcctcgcccac

SEQ ID NO: 51 - DNA, 1011 bp, Aeromonas salmonicida
atgaaaaaatggtttgtttgtttattggggttgatcgcgctgacagttcaggcagccgacactcgccccgccttctcccggatcgtgatgttcggcgacagcctctccgataccggcaaaatgtacagcaagatgcgcggttacctcccctccagcccgccctactatgagggccgtttctccaacggacccgtctggctggagcagctgaccaagcagttcccgggtctgaccatcgccaacgaagcggaaggcggtgccactgccgtggcttacaacaagatctcctggaatcccaagtatcaggtctacaacaacctggactacgaggtcacccagttcttgcagaaagacagcttcaagccggacgatctggtgatcctctgggtcggtgccaatgactatctggcatatggctggaatacggagcaggatgccaagcgagttcgcgatgccatcagcgatgcggccaaccgcatggtactgaacggtgccaagcagatactgctgttcaacctgccggatctgggccagaacccgtcagcccgcagtcagaaggtggtcgaggcggtcagccatgtctccgcctatcacaacaagctgctgctgaacctggcacgccagctggcccccaccggcatggtaaagctgttcgagatcgacaagcaatttgccgagatgctgcgtgatccgcagaacttcggcctgagcgacgtcgagaacccctgctacgacggcggctatgtgtggaagccgtttgccacccgcagcgtcagcaccgaccgccagctctccgccttcagtccgcaggaacgcctcgccatcgccggcaacccgctgctggcacaggccgttgccagtcctatggcccgccgcagcgccagccccctcaactgtgagggcaagatgttctgggatcaggtacacccgaccactgtcgtgcacgcagccctgagcgagcgcgccgccaccttcatcgagacccagtacgagttcctcgcccacggatga

SEQ ID NO: 52 - DNA, 888 bp, Streptomyces coelicolor
atgccgaagcctgcccttcgccgtgtcatgaccgcgacagtcgccgccgtcggcacgctcgccctcggcctcaccgacgccaccgcccacgccgcgcccgcccaggccactccgaccctggactacgtcgccctcggcgacagctacagcgccggctccggcgtcctgcccgtcgaccccgccaacctgctctgtctgcgctcgacggccaactacccccacgtcatcgcggacacgacgggcgcccgcctcacggacgtcacctgcggcgccgcgcagaccgccgacttcacgcgggcccagtacccgggcgtcgcaccccagttggacgcgctcggcaccggcacggacctggtcacgctcaccatcggcggcaacgacaacagcaccttcatcaacgccatcacggcctgcggcacggcgggtgtcctcagcggcggcaagggcagcccctgcaaggacaggcacggcacctccttcgacgacgagatcgaggccaacacgtaccccgcgctcaaggaggcgctgctcggcgtccgcgccagggctccccacgccagggtggcggctctcggctacccgtggatcaccccggccaccgccgacccgtcctgcttcctgaagctccccctcgccgccggtgacgtgccctacctgcgggccatccaggcacacctcaacgacgcggtccggcgggccgccgaggagaccggagccacctacgtggacttctccggggtgtccgacggccacgacgcctgcgaggcccccggcacccgctggatcgaaccgctgctcttcgggcacagcctcgttcccgtccaccccaacgccctgggcgagcggcgcatggccgagcacacgatggacgtcctcggcctggactga

SEQ ID NO: 53 - DNA, 888 bp, Streptomyces coelicolor
tcagtccaggccgaggacgtccatcgtgtgctcggccatgcgccgctcgcccagggcgttggggtggacgggaacgaggctgtgcccgaagagcagcggttcgatccagcgggtgccgggggcctcgcaggcgtcgtggccgtcggacaccccggagaagtccacgtaggtggctccggtctcctcggcggcccgccggaccgcgtcgttgaggtgtgcctggatggcccgcaggtagggcacgtcaccggcggcgagggggagcttcaggaagcaggacgggtcggcggtggccggggtgatccacgggtagccgagagccgccaccctggcgtggggagccctggcgcggacgccgagcagcgcctccttgagcgcggggtacgtgttggcctcgatctcgtcgtcgaaggaggtgccgtgcctgtccttgcaggggctgcccttgccgccgctgaggacacccgccgtgccgcaggccgtgatggcgttgatgaaggtgctgttgtcgttgccgccgatggtgagcgtgaccaggtccgtgccggtgccgagcgcgtccaactggggtgcgacgcccgggtactgggcccgcgtgaagtcggcggtctgcgcggcgccgcaggtgacgtccgtgaggcgggcgcccgtcgtgtccgcgatgacgtgggggtagttggccgtcgagcgcagacagagcaggttggcggggtcgacgggcaggacgccggagccggcgctgtagctgtcgccgagggcgacgtagtccagggtcggagtggcctgggcgggcgcggcgtgggcggtggcgtcggtgaggccgagggcgagcgtgccgacggcggcgactgtcgcggtcatgacacggcgaagggcaggcttcggcat

SEQ ID NO: 54 - DNA, 717 bp, Saccharomyces cerevisiae
atggattacgagaagtttctgttatttggggattccattactgaatttgcttttaatactaggcccattgaagatggcaaagatcagtatgctcttggagccgcattagtcaacgaatatacgagaaaaatggatattcttcaaagagggttcaaagggtacacttctagatgggcgttgaaaatacttcctgagattttaaagcatgaatccaatattgtcatggccacaatatttttgggtgccaacgatgcatgctcagcaggtccccaaagtgtccccctccccgaatttatcgataatattcgtcaaatggtatctttgatgaagtcttaccatatccgtcctattataataggaccggggctagtagatagagagaagtgggaaaaagaaaaatctgaagaaatagctctcggatacttccgtaccaacgagaactttgccatttattccgatgccttagcaaaactagccaatgaggaaaaagttcccttcgtggctttgaataaggcgtttcaacaggaaggtggtgatgcttggcaacaactgctaacagatggactgcacttttccggaaaagggtacaaaatttttcatgacgaattattgaaggtcattgagacattctacccccaatatcatcccaaaaacatgcagtacaaactgaaagattggagagatgtgctagatgatggatctaacataatgtcttga

SEQ ID NO: 55 - DNA, 1044 bp, Ralstonia sp.
atgaacctgcgtcaatggatgggcgccgccacggctgcccttgccttgggcttggccgcgtgcgggggcggtgggaccgaccagagcggcaatcccaatgtcgccaaggtgcagcgcatggtggtgttcggcgacagcctgagcgatatcggcacctacacccccgtcgcgcaggcggtgggcggcggcaagttcaccaccaacccgggcccgatctgggccgagaccgtggccgcgcaactgggcgtgacgctcacgccggcggtgatgggctacgccacctccgtgcagaattgccccaaggccggctgcttcgactatgcgcagggcggctcgcgcgtgaccgatccgaacggcatcggccacaacggcggcgcgggggcgctgacctacccggttcagcagcagctcgccaacttctacgcggccagcaacaacacattcaacggcaataacgatgtcgtcttcgtgctggccggcagcaacgacattttcttctggaccactgcggcggccaccagcggctccggcgtgacgcccgccattgccacggcccaggtgcagcaggccgcgacggacctggtcggctatgtcaaggacatgatcgccaagggtgcgacgcaggtctacgtgttcaacctgcccgacagcagcctgacgccggacggcgtggcaagcggcacgaccggccaggcgctgctgcacgcgctggtgggcacgttcaacacgacgctgcaaagcgggctggccggcacctcggcgcgcatcatcgacttcaacgcacaactgaccgcggcgatccagaatggcgcctcgttcggcttcgccaacaccagcgcccgggcctgcgacgccaccaagatcaatgccctggtgccgagcgccggcggcagctcgctgttctgctcggccaacacgctggtggcttccggtgcggaccagagctacctgttcgccgacggcgtgcacccgaccacggccggccatcgcctgatcgccagcaacgtgctggcgcgcctgctggcggataacgtcgcgcactga

SEQ ID NO: 56 - DNA, 786 bp, Streptomyces coelicolor
gtgatcgggtcgtacgtggcggtgggggacagcttcaccgagggcgtcggcgaccccggccccgacggggcgttcgtcggctgggccgaccggctcgccgtactgctcgcggaccggcgccccgagggcgacttcacgtacacgaacctcgccgtgcgcggcaggctcctcgaccagatcgtggcggaacaggtcccgcgggtcgtcggactcgcgcccgacctcgtctcgttcgcggcgggcggcaacgacatcatccggcccggcaccgatcccgacgaggtcgccgagcggttcgagctggcggtggccgcgctgaccgccgcggccggaaccgtcctggtgaccaccgggttcgacacccggggggtgcccgtcctcaagcacctgcgcggcaagatcgccacgtacaacgggcacgtccgcgccatcgccgaccgctacggctgcccggtgctcgacctgtggtcgctgcggagcgtccaggaccgcagggcgtgggacgccgaccggctgcacctgtcgccggaggggcacacccgggtggcgctgcgcgcggggcaggccctgggcctgcgcgtcccggccgaccctgaccagccctggccgcccctgccgccgcgcggcacgctcgacgtccggcgcgacgacgtgcactgggcgcgcgagtacctggtgccgtggatcgggcgccggctgcggggcgagtcgtcgggcgaccacgtgacggccaaggggacgctgtcgccggacgccatcaagacgcggatcgccgcggtggcctga

SEQ ID NO: 57 - DNA, 783 bp, Streptomyces coelicolor
atgcagacgaaccccgcgtacaccagtctcgtcgccgtcggcgactccttcaccgagggcatgtcggacctgctgcccgacggctcctaccgtggctgggccgacctcctcgccacccggatggcggcccgctcccccggcttccggtacgccaacctggcggtgcgcgggaagctgatcggacagatcgtcgacgagcaggtggacgtggccgccgccatgggagccgacgtgatcacgctggtcggcgggctcaacgacacgctgcggcccaagtgcgacatggcccgggtgcgggacctgctgacccaggccgtggaacggctcgccccgcactgcgagcagctggtgctgatgcgcagtcccggtcgccagggtccggtgctggagcgcttccggccccgcatggaggccctgttcgccgtgatcgacgacctggccgggcggcacggcgccgtggtcgtcgacctgtacggggcccagtcgctggccgaccctcggatgtgggacgtggaccggctgcacctgaccgccgagggccaccgccgggtcgcggaggcggtgtggcagtcgctcggccacgagcccgaggaccccgagtggcacgcgccgatcccggcgacgccgccgccggggtgggtgacgcgcaggaccgcggacgtccggttcgcccggcagcacctgctgccctggataggccgcaggctgaccgggcgctcgtccggggacggcctgccggccaagcgcccggacctgctgccctacgaggaccccgcacggtga

SEQ ID NO: 58 - DNA, 1365 bp, Streptomyces coelicolor
atgacccggggtcgtgacgggggtgcgggggcgccccccaccaagcaccgtgccctgctcgcggcgatcgtcaccctgatagtggcgatctccgcggccatatacgccggagcgtccgcggacgacggcagcagggaccacgcgctgcaggccggaggccgtctcccacgaggagacgccgcccccgcgtccaccggtgcctgggtgggcgcctgggccaccgcaccggccgcggccgagccgggcaccgagacgaccggcctggcgggccgctccgtgcgcaacgtcgtgcacacctcggtcggcggcaccggcgcgcggatcaccctctcgaacctgtacgggcagtcgccgctgaccgtcacacacgcctcgatcgccctggccgccgggcccgacaccgccgccgcgatcgccgacaccatgcgccggctcaccttcggcggcagcgcccgggtgatcatcccggcgggcggccaggtgatgagcgacaccgcccgcctcgccatcccctacggggcgaacgtcctggtcaccacgtactcccccatcccgtccgggccggtgacctaccatccgcaggcccggcagaccagctacctggccgacggcgaccgcacggcggacgtcaccgccgtcgcgtacaccacccccacgccctactggcgctacctgaccgccctcgacgtgctgagccacgaggccgacggcacggtcgtggcgttcggcgactccatcaccgacggcgcccgctcgcagagcgacgccaaccaccgctggaccgacgtcctcgccgcacgcctgcacgaggcggcgggcgacggccgggacacgccccgctacagcgtcgtcaacgagggcatcagcggcaaccggctcctgaccagcaggccggggcggccggccgacaacccgagcggactgagccggttccagcgggacgtgctggaacgcaccaacgtcaaggccgtcgtcgtcgtcctcggcgtcaacgacgtcctgaacagcccggaactcgccgaccgcgacgccatcctgaccggcctgcgcaccctcgtcgaccgggcgcacgcccggggactgcgggtcgtcggcgccacgatcacgccgttcggcggctacggcggctacaccgaggcccgcgagacgatgcggcaggaggtcaacgaggagatccgctccggccgggtcttcgacacggtcgtcgacttcgacaaggccctgcgcgacccgtacgacccgcgccggatgcgctccgactacgacagcggcgaccacctgcaccccggcgacaaggggtacgcgcgcatgggcgcggtcatcgacctggccgcgctgaagggcgcggcgccggtcaaggcgtag

SEQ ID NO: 59 - DNA, 1023 bp, Streptomyces coelicolor
atgacgagcatgtcgagggcgagggtggcgcggcggatcgcggccggcgcggcgtacggcggcggcggcatcggcctggcgggagcggcggcggtcggtctggtggtggccgaggtgcagctggccagacgcagggtgggggtgggcacgccgacccgggtgccgaacgcgcagggactgtacggcggcaccctgcccacggccggcgacccgccgctgcggctgatgatgctgggcgactccacggccgccgggcagggcgtgcaccgggccgggcagacgccgggcgcgctgctggcgtccgggctcgcggcggtggcggagcggccggtgcggctggggtcggtcgcccagccgggggcgtgctcggacgacctggaccggcaggtggcgctggtgctcgccgagccggaccgggtgcccgacatctgcgtgatcatggtcggcgccaacgacgtcacccaccggatgccggcgacccgctcggtgcggcacctgtcctcggcggtacggcggctgcgcacggccggtgcggaggtggtggtcggcacctgtccggacctgggcacgatcgagcgggtgcggcagccgctgcgctggctggcccggcgggcctcacggcagctcgcggcggcacagaccatcggcgccgtcgagcagggcgggcgcacggtgtcgctgggcgacctgctgggtccggagttcgcgcagaacccgcgggagctcttcggccccgacaactaccacccctccgccgaggggtacgccacggccgcgatggcggtactgccctcggtgtgcgccgcgctcggcctgtggccggccgacgaggagcacccggacgcgctgcgccgcgagggcttcctgccggtggcgcgcgcggcggcggaggcggcgtccgaggcgggtacggaggtcgccgccgccatgcctacggggcctcgggggccctgggcgctgctgaagcgccggagacggcgtcgggtgtcggaggcggaaccgtccagcccgtccggcgtttga

SEQ ID NO: 60 - DNA, 918 bp, Streptomyces coelicolor
atgggtcgagggacggaccagcggacgcggtacggccgtcgccgggcgcgtgtcgcgctcgccgccctgaccgccgccgtcctgggcgtgggcgtggcgggctgcgactccgtgggcggcgactcacccgctccttccggcagcccgtcgaagcggacgaggacggcgcccgcctgggacaccagcccggcgtccgtcgccgccgtgggcgactccatcacgcgcggcttcgacgcctgtgcggtgctgtcggactgcccggaggtgtcgtgggcgaccggcagcagcgcgaaggtcgactcgctggccgtacggctgctggggaaggcggacgcggccgagcacagctggaactacgcggtcaccggggcccggatggcggacctgaccgctcaggtgacgcgggcggcgcagcgcgagccggagctggtggcggtgatggccggggcgaacgacgcgtgccggtccacgacctcggcgatgacgccggtggcggacttccgggcgcagttcgaggaggcgatggccaccctgcgcaagaagctccccaaggcgcaggtgtacgtgtcgagcatcccggacctcaagcggctctggtcccagggccgcaccaacccgctgggcaagcaggtgtggaagctcggcctgtgcccgtcgatgctgggcgacgcggactccctggactcggcggcgaccctgcggcgcaacacggtgcgcgaccgggtggcggactacaacgaggtgctgcgggaggtctgcgcgaaggaccggcggtgccgcagcgacgacggcgcggtgcacgagttccggttcggcacggaccagttgagccactgggactggttccacccgagtgtggacggccaggcccggctggcggagatcgcctaccgcgcggtcaccgcgaagaatccctga

SEQ ID NO: 61 - DNA, 1068 bp, Streptomyces rimosus
ttcatcacaacgatgtcacaacaccggccatccgggtcatccctgatcgtgggaatgggtgacaagccttcccgtgacgaaagggtcctgctacatcagaaatgacagaaatcctgctcagggaggttccatgagactgtcccgacgcgcggccacggcgtccgcgctcctcctcaccccggcgctcgcgctcttcggcgcgagcgccgccgtgtccgcgccgcgaatccaggccaccgactacgtggccctcggcgactcctactcctcgggggtcggcgcgggcagctacgacagcagcagtggctcctgtaagcgcagcaccaagtcctacccggccctgtgggccgcctcgcacaccggtacgcggttcaacttcaccgcctgttcgggcgcccgcacaggagacgtgctggccaagcagctgaccccggtcaactccggcaccgacctggtcagcattaccatcggcggcaacgacgcgggcttcgccgacaccatgaccacctgcaacctccagggcgagagcgcgtgcctggcgcggatcgccaaggcgcgcgcctacatccagcagacgctgcccgcccagctggaccaggtctacgacgccatcgacagccgggcccccgcagcccaggtcgtcgtcctgggctacccgcgcttctacaagctgggcggcagctgcgccgtcggtctctcggagaagtcccgcgcggccatcaacgccgccgccgacgacatcaacgccgtcaccgccaagcgcgccgccgaccacggcttcgccttcggggacgtcaacacgaccttcgccgggcacgagctgtgctccggcgccccctggctgcacagcgtcacccttcccgtggagaactcctaccaccccacggccaacggacagtccaagggctacctgcccgtcctgaactccgccacctgatctcgcggctactccgcccctgacgaagtcccgcccccgggcggggcttcgccgtaggtgcgcgtaccgccgtcgcccgtcgcgccggtggccccgccgtacgtgccgccgcccccggacgcggtcggttc

SEQ ID NO: 62 - DNA, 1008 bp, Aeromonas hydrophila
atgaaaaaatggtttgtgtgtttattgggattggtcgcgctgacagttcaggcagccgacagtcgccccgccttttcccggatcgtgatgttcggcgacagcctctccgataccggcaaaatgtacagcaagatgcgcggttacctcccctccagcccgccctactatgagggccgtttctccaacggacccgtctggctggagcagctgaccaaacagttcccgggtctgaccatcgccaacgaagcggaaggcggtgccactgccgtggcttacaacaagatctcctggaatcccaagtatcaggtcatcaacaacctggactacgaggtcacccagttcttgcagaaagacagcttcaagccggacgatctggtgatcctctgggtcggtgccaatgactatctggcctatggctggaacacggagcaggatgccaagcgggttcgcgatgccatcagcgatgcggccaaccgcatggtactgaacggtgccaagcagatactgctgttcaacctgccggatctgggccagaacccgtcagctcgcagtcagaaggtggtcgaggcggtcagccatgtctccgcctatcacaaccagctgctgctgaacctggcacgccagctggcccccaccggcatggtaaagctgttcgagatcgacaagcaatttgccgagatgctgcgtgatccgcagaacttcggcctgagcgacgtcgagaacccctgctacgacggcggctatgtgtggaagccgtttgccacccgcagcgtcagcaccgaccgccagctctccgccttcagtccgcaggaacgcctcgccatcgccggcaacccgctgctggcacaggccgttgccagtcctatggcccgccgcagcgccagccccctcaactgtgagggcaagatgttctgggatcaggtacacccgaccactgtcgtgcacgcagccctgagcgagcgcgccgccaccttcatcgcgaaccagtacgagttcctcgcccactga

SEQ ID NO: 63 - DNA, 1011 bp, Aeromonas salmonicida
atgaaaaaatggtttgtttgtttattggggttgatcgcgctgacagttcaggcagccgacactcgccccgccttctcccggatcgtgatgttcggcgacagcctctccgataccggcaaaatgtacagcaagatgcgcggttacctcccctccagcccgccctactatgagggccgtttctccaacggacccgtctggctggagcagctgaccaagcagttcccgggtctgaccatcgccaacgaagcggaaggcggtgccactgccgtggcttacaacaagatctcctggaatcccaagtatcaggtcatcaacaacctggactacgaggtcacccagttcttgcagaaagacagcttcaagccggacgatctggtgatcctctgggtcggtgccaatgactatctggcatatggctggaatacggagcaggatgccaagcgagttcgcgatgccatcagcgatgcggccaaccgcatggtactgaacggtgccaagcagatactgctgttcaacctgccggatctgggccagaacccgtcagcccgcagtcagaaggtggtcgaggcggtcagccatgtctccgcctatcacaacaagctgctgctgaacctggcacgccagctggcccccaccggcatggtaaagctgttcgagatcgacaagcaatttgccgagatgctgcgtgatccgcagaacttcggcctgagcgacgtcgagaacccctgctacgacggcggctatgtgtggaagccgtttgccacccgcagcgtcagcaccgaccgccagctctccgccttcagtccgcaggaacgcctcgccatcgccggcaacccgctgctggcacaggccgttgccagtcctatggcccgccgcagcgccagccccctcaactgtgagggcaagatgttctgggatcaggtacacccgaccactgtcgtgcacgcagccctgagcgagcgcgccgccaccttcatcgagacccagtacgagttcctcgcccacggatga

SEQ ID NO: 64 - protein, 8 aa, Artificial Sequence
  Description of Artificial Sequence Synthetic peptide
MRRSRFLA

SEQ ID NO: 65 - protein, 8 aa, Artificial Sequence
  Description of Artificial Sequence Synthetic peptide
ALILLTLA

SEQ ID NO: 66 - protein, 5 aa, Artificial Sequence
  Description of Artificial Sequence Synthetic peptide
ARAAP

SEQ ID NO: 67 - protein, 11 aa, Artificial Sequence
  Description of Artificial Sequence Synthetic peptide
YVALGDSYSSG

SEQ ID NO: 68 - protein, 280 aa, Aeromonas salmonicida
ADTRPAFSRIVMFGDSLSDTGKMYSKMRGYLPSSPPYYEGRFSNGPVWLEQLTKQFPGLTIANEAEGGATAVAYNKISWDPKYQVINNLDYEVTQFLQKDSFKPDDLVILWVGANDYLAYGWNTEQDAKRVRDAISDAANRMVLNGAKQILLFNLPDLGQNPSARSQKVVEAVSHVSAYHNKLLLNLARQLAPTGMVKLFEIDKQFAEMLRDPQNFGLSDVENPCYDGGYVWKPFRSASPLNCEGKMFWDQVHPTTVVHAALSERAATFIETQYEFLAHG

SEQ ID NO: 69 - DNA, 954 bp, Aeromonas salmonicida
gacactcgccccgccttctcccggatcgtgatgttcggcgacagcctctccgataccggcaaaatgtacagcaagatgcgcggttacctcccctccagcccgccctactatgagggccgtttctccaacggacccgtctggctggagcagctgaccaagcagttcccgggtctgaccatcgccaacgaagcggaaggcggtgccactgccgtggcttacaacaagatctcctgggaccccaagtatcaggtcatcaacaacctggactacgaggtcacccagttcttgcagaaagacagcttcaagccggacgatctggtgatcctctgggtcggtgccaatgactatctggcatatggctggaatacggagcaggatgccaagcgagttcgcgatgccatcagcgatgcggccaaccgcatggtactgaacggtgccaagcagatactgctgttcaacctgccggatctgggccagaacccgtcagcccgcagtcagaaggtggtcgaggcggtcagccatgtctccgcctatcacaacaagctgctgctgaacctggcacgccagctggcccccaccggcatggtaaagctgttcgagatcgacaagcaatttgccgagatgctgcgtgatccgcagaacttcggcctgagcgacgtcgagaacccctgctacgacggcggctatgtgtggaagccgtttgccacccgcagcgtcagcaccgaccgccagctctccgccttcagtccgcaggaacgcctcgccatcgccggcaacccgctgctggcacaggccgttgccagtcctatggcccgccgcagcgccagccccctcaactgtgagggcaagatgttctgggatcaggtacacccgaccactgtcgtgcacgcagccctgagcgagcgcgccgccaccttcatcgagacccagtacgagttcctcgcccacggatga

SEQ ID NO: 70 - protein, 279 aa, Aeromonas salmonicida
ADTRPAFSRIVMFGDSLSDTGKMYSKMRGYLPSSPPYYEGRFSNGPVWLEQLTKQFPGLTIANEAEGGATAVAYNKISWDPKYQVINNLDYEVTQFLQKDSFKPDDLVILWVGANDYLAYGWNTEQDAKRVRDAISDAANRMVLNGAKQILLFNLPDLGQNPSARSQKVVEAVSHVSAYHNKLLLNLARQLAPTGMVKLFEIDKQFAEMLRDPQNFGLSDVENPCYDGGYVWKPFSASPLNCEGKMFWDQVHPTTVVHAALSERAATFIETQYEFLAHG

SEQ ID NO: 71 - protein, 278 aa, Aeromonas salmonicida
ADTRPAFSRIVMFGDSLSDTGKMYSKMRGYLPSSPPYYEGRFSNGPVWLEQLTKQFPGLTIANEAEGGATAVAYNKISWDPKYQVINNLDYEVTQFLQKDSFKPDDLVILWVGANDYLAYGWNTEQDAKRVRDAISDAANRMVLNGAKQILLFNLPDLGQNPSARSQKVVEAVSHVSAYHNKLLLNLARQLAPTGMVKLFEIDKQFAEMLRDPQNFGLSDVENPCYDGGYVWKPFASPLNCEGKMFWDQVHPTTVVHAALSERAATFIETQYEFLAHG

SEQ ID NO: 72 - protein, 277 aa, Aeromonas salmonicida
ADTRPAFSRIVMFGDSLSDTGKMYSKMRGYLPSSPPYYEGRFSNGPVWLEQLTKQFPGLTIANEAEGGATAVAYNKISWDPKYQVINNLDYEVTQFLQKDSFKPDDLVILWVGANDYLAYGWNTEQDAKRVRDAISDAANRMVLNGAKQILLFNLPDLGQNPSARSQKVVEAVSHVSAYHNKLLLNLARQLAPTGMVKLFEIDKQFAEMLRDPQNFGLSDVENPCYDGGYVWKPFSPLNCEGKMFWDQVHPTTVVHAALSERAATFIETQYEFLAHG

SEQ ID NO: 73 - protein, 79 aa, Aeromonas salmonicida
AEMLRDPQNFGLSDVENPCYDGGYVWKPFATRSVSTDRQLSASPQERLAIAGNPLLAQAVASPMARRSASPLNCEGKMF

SEQ ID NO: 74 - protein, 5 aa, Artificial Sequence
  Description of Artificial Sequence Synthetic peptide
GAGSY

SEQ ID NO: 75 - protein, 4 aa, Artificial Sequence
  Description of Artificial Sequence Synthetic peptide
SSGD

SEQ ID NO: 76 - protein, 15 aa, Artificial Sequence
  Description of Artificial Sequence Synthetic peptide
RSTKAYPALWAAAHA

SEQ ID NO: 77 - protein, 5 aa, Artificial Sequence
  Description of Artificial Sequence Synthetic peptide
SSFSF

SEQ ID NO: 78 - protein, 12 aa, Artificial Sequence
  Description of Artificial Sequence Synthetic peptide
ACSGARTYDVLA

SEQ ID NO: 79 - protein, 15 aa, Artificial Sequence
  Description of Artificial Sequence Synthetic peptide
LVSITIGGNDAGFAD

SEQ ID NO: 80 - protein, 6 aa, Artificial Sequence
  Description of Artificial Sequence Synthetic peptide
MTTCVL

SEQ ID NO: 81 - protein, 6 aa, Artificial Sequence
  Description of Artificial Sequence Synthetic peptide
SDSACL

SEQ ID NO: 82 - protein, 4 aa, Artificial Sequence
  Description of Artificial Sequence Synthetic peptide
TLPA

SEQ ID NO: 83 - protein, 9 aa, Artificial Sequence
  Description of Artificial Sequence Synthetic peptide
RLDSVYSAI

SEQ ID NO: 84 - protein, 4 aa, Artificial Sequence
  Description of Artificial Sequence Synthetic peptide
TRAP

SEQ ID NO: 85 - protein, 12 aa, Artificial Sequence
  Description of Artificial Sequence Synthetic peptide
ARVVVLGYPRIY

SEQ ID NO: 86 - protein, 4 aa, Artificial Sequence
  Description of Artificial Sequence Synthetic peptide
LGLS

SEQ ID NO: 87 - protein, 11 aa, Artificial Sequence
  Description of Artificial Sequence Synthetic peptide
TKRAAINDAAD

SEQ ID NO: 88 - protein, 12 aa, Artificial Sequence
  Description of Artificial Sequence Synthetic peptide
LNSVIAKRAADH

SEQ ID NO: 89 - protein, 7 aa, Artificial Sequence
  Description of Artificial Sequence Synthetic peptide
GFTFGDV

SEQ ID NO: 90 - protein, 7 aa, Artificial Sequence
  Description of Artificial Sequence Synthetic peptide
GHELCSA

SEQ ID NO: 91 - protein, 9 aa, Artificial Sequence
  Description of Artificial Sequence Synthetic peptide
PWLHSLTLP

SEQ ID NO: 92 - protein, 6 aa, Artificial Sequence
  Description of Artificial Sequence Synthetic peptide
SYHPTA

SEQ ID NO: 93 - protein, 13 aa, Artificial Sequence
  Description of Artificial Sequence Synthetic peptide
GHAAGYLPVLNSI

SEQ ID NO: 94 - protein, 230 aa, Aspergillus aculeatus
TTVYLAGDSTMAKNGGGSGTNGWGEYLASYLSATVVNDAVAGRSARSYTREGRFENIADVVTAGDYVIVEFGHNDGGSLSTDNGRTDCSGTGAEVCYSVYDGVNETILTFPAYLENAAKLFTAKGAKVILSSQTPNNPWETGTFVNSPTRFVEYAELAAEVAGVEYVDHWSYVDSIYETLGNATVNSYFPIDHTHTSPAGAEVVAEAFLKAVVCTGTSLKSVLTTTSFEG

SEQ ID NO: 95 - protein, 184 aa, Escherichia coli
ADTLLILGDSLSAGYRMSASAAWPALLNDKWQSKTSVVNASISGDTSQQGLARLPALLKQHQPRWVLVELGGNDGLRGFQPQQTEQTLRQILQDVKAANAEPLLMQIRLPANYGRRYNEAFSAIYPKLAKEFDVPLLPFFMEEVYLKPQWMQDDGIHPNRDAQPFIADWMAKQLQPLVNHDSLE

SEQ ID NO: 96 - protein, 308 aa, Aeromonas hydrophila
IVMFGDSLSDTGKMYSKMRGYLPSSPPYYEGRFSNGPVWLEQLTNEFPGLTIANEAEGGPTAVAYNKISWNPKYQVINNLDYEVTQFLQKDSFKPDDLVILWVGANDYLAYGWNTEQDAKRVRDAISDAANRMVLNGAKEILLFNLPDLGQNPSARSQKVVEAASHVSAYHNQLLLNLARQLAPTGMVKLFEIDKQFAEMLRDPQNFGLSDQRNACYGGSYVWKPFASRSASTDSQLSAFNPQERLAIAGNPLLAQAVASPMAARSASTLNCEGKMFWDQVHPTTVVHAALSEPAATFIESQYEFLAH

SEQ ID NO: 97 - protein, 232 aa, Aspergillus aculeatus
TTVYLAGDSTMAKNGGGSGTNGWGEYLASYLSATVVNDAVAGRSARSYTREGRFENIADVVTAGDYVIVEFGHNDGGSLSTDNGRTDCSGTGAEVCYSVYDGVNETILTFPAYLENAAKLFTAKGAKVILSSQTPNNPWETGTFVNSPTRFVEYAELAAEVAGVEYVDHWSYVDSIYETLGNATVNSYFPIDHTHTSPAGAEVVAEAFLKAVVCTGTSLKSVLTTTSFEGTC

SEQ ID NO: 98 - protein, 167 aa, Escherichia coli
LLILGDSLSAGYRMSASAAWPALLNDKWQSKTSVVNASISGDTSQQGLARLPALLKQHQPRWVLVELGGNDGLRGFQPQQTEQTLRQILQDVKAANAEPLLMQIRLPANYGRRYNEAFSAIYPKLAKEFDVPLLPFFMEEVYLKPQWMQDDGIHPNRDAQPFIADWM

SEQ ID NO: 99 - protein, 295 aa, Aeromonas hydrophila
IVMFGDSLSDTGKMYSKMRGYLPSSPPYYEGRFSNGPVWLEQLTNEFPGLTIANEAEGGPTAVAYNKISWNPKYQVINNLDYEVTQFLQKDSFKPDDLVILWVGANDYLAYGWNTEQDAKRVRDAISDAANRMVLNGAKEILLFNLPDLGQNPSARSQKVVEAASHVSAYHNQLLLNLARQLAPTGMVKLFEIDKQFAEMLRDPQNFGLSDQRNACYGGSYVWKPFASRSASTDSQLSAFNPQERLAIAGNPLLAQAVASPMAARSASTLNCEGKMFWDQVHPTTVVHAALSEPA

SEQ ID NO: 100 - protein, 50 aa, Artificial Sequence
  Description of Artificial Sequence Synthetic polypeptide
RPAFSRIVMFGDSLSDTGKMYSKMRGYLPSSPPYYEGRFSNGPVWLEQLT

SEQ ID NO: 101 - protein, 13 aa, Artificial Sequence
  Description of Artificial Sequence Synthetic peptide
FPGLTIANEAEGG

SEQ ID NO: 102 - protein, 79 aa, Artificial Sequence
  Description of Artificial Sequence Synthetic polypeptide
TAVAYNKISWNPKYQVINNLDYEVTQFLQKDSFKPDDLVILWVGANDYLAYGWNTEQDAKRVRDAISDAANRMVLNGAK

SEQ ID NO: 103 - protein, 23 aa, Artificial Sequence
  Description of Artificial Sequence Synthetic peptide
ILLFNLPDLGQNPSARSQKVVEA

SEQ ID NO: 104 - protein, 8 aa, Artificial Sequence
  Description of Artificial Sequence Synthetic peptide
SHVSAYHN

SEQ ID NO: 105 - protein, 38 aa, Artificial Sequence
  Description of Artificial Sequence Synthetic polypeptide
LLLNLARQLAPTGMVKLFEIDKQFAEMLRDPQNFGLSD

SEQ ID NO: 106 - protein, 7 aa, Artificial Sequence
  Description of Artificial Sequence Synthetic peptide
YVWKPFA

SEQ ID NO: 107 - protein, 5 aa, Artificial Sequence
  Description of Artificial Sequence Synthetic peptide
QLSAF

SEQ ID NO: 108 - protein, 22 aa, Artificial Sequence
  Description of Artificial Sequence Synthetic peptide
PQERLAIAGNPLLAQAVASPMA

SEQ ID NO: 109 - protein, 4 aa, Artificial Sequence
  Description of Artificial Sequence Synthetic peptide
RSAS

SEQ ID NO: 110 - protein, 24 aa, Artificial Sequence
  Description of Artificial Sequence Synthetic peptide
LNCEGKMFWDQVHPTTVVHAALSE

SEQ ID NO: 111 - protein, 5 aa, Artificial Sequence
  Description of Artificial Sequence Synthetic peptide
AATFI

SEQ ID NO: 112 - protein, 7 aa, Artificial Sequence
  Description of Artificial Sequence Synthetic peptide
QYEFLAH

SEQ ID NO: 113 - protein, 5 aa, Artificial Sequence
  Description of Artificial Sequence Synthetic peptide
GANDY

SEQ ID NO: 114 - protein, 4 aa, Artificial Sequence
  Description of Artificial Sequence Synthetic peptide
GDSL

SEQ ID NO: 115 - protein, 5 aa, Artificial Sequence
  Description of Artificial Sequence Synthetic peptide
GGNDA

SEQ ID NO: 116 - protein, 5 aa, Artificial Sequence
  Description of Artificial Sequence Synthetic peptide
GGNDL

SEQ ID NO: 117 - protein, 4 aa, Artificial Sequence
  Description of Artificial Sequence Synthetic peptide
GDSY

SEQ ID NO: 118 - protein, 317 aa, Aeromonas hydrophila
ADSRPAFSRIVMFGDSLSDTGKMYSKMRGYLPSSPPYYEGRFSNGPVWLEQLTKQFPGLTIANEAEGGATAVAYNKISWNPKYQVINNLDYEVTQFLQKDSFKPDDLVILWVGANDYLAYGWNTEQDAKRVRDAISDAANRMVLNGAKQILLFNLPDLGQNPSARSQKVVEAVSHVSAYHNQLLLNLARQLAPTGMVKLFEIDKQFAEMLRDPQNFGLSDVENPCYDGGYVWKPFATRSVSTDRQLSAFSPQERLAIAGNPLLAQAVASPMARRSASPLNCEGKMFWDQVHPTTVVHAALSERAATFIANQYEFLAH